(19) United States
(12) Patent Application Publication
(10) Pub. No.: US 2007/0232532 A1
(43) Pub. Date: Oct. 4, 2007

(54) Use of cyclosporin alkene analogues for preventing or treating viral-induced disorders

(75) Inventor: Bruce F. Molino, Slingerlands, NY (US)

Correspondence Address: NIXON PEABODY LLP - PATENT GROUP, CLINTON SQUARE, P.O. BOX 31051, ROCHESTER, NY, 14603-1051, US

(73) Assignee: AMR Technology, Inc., Manchester Center, VT (US)

(21) Appl. No.: 11/391,027

(22) Filed: Mar. 28, 2006

(51) Int. Cl. A61K 38/13

(52) U.S. Cl. 514/11

(57) ABSTRACT

The present invention relates to methods of preventing or treating a mammal with a viral-induced disorder. The method involves administering to the mammal a therapeutically effective amount of a compound represented by Formula I, as shown below: 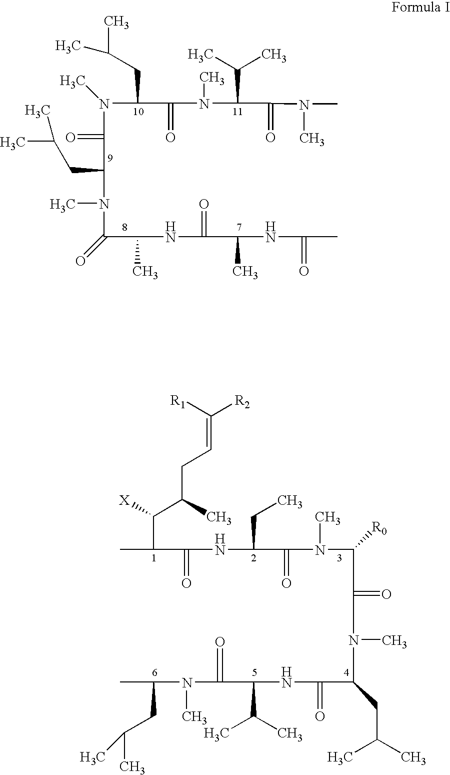
or a pharmaceutically acceptable salt thereof, with X, R₀, R₁, and R₂ defined herein, under conditions effective to prevent or treat the viral-induced disorder.

### FIELD OF THE INVENTION

The present invention discloses novel cyclosporin analogues and their utilities as pharmaceutical agents for prevention and treatment of viral-induced diseases. Methods for preparation of such analogues are also disclosed.

### BACKGROUND OF THE INVENTION

Cyclosporin A (CsA), a neutral cyclic undecapeptide isolated from the fungus Tolypocladium inflatum and currently marketed as Neoral® and Sandimmune® (Novartis, Basel, Switzerland), has been widely used for the prevention of organ transplant rejection. The molecular basis for the immunosuppressant activity of cyclosporin A and cyclosporin analogues begins with the passive diffusion of the cyclosporin (Cs) molecule into the cell, followed by binding to its intracellular receptor, cyclophilin A (CypA). CypA belongs to a family of proteins that catalyze cis-trans peptidyl-prolyl isomerization, i.e., PPIase, a rate-limiting step in protein folding. CsA and other cyclosporin analogues bind to the active site of CypA. However, immunosuppression is not believed to be due to the inhibition of CypA PPIase activity. The target of the CsA-CypA complex is a Ca²⁺-calmodulin-dependent serine-threonine-specific protein phosphatase, calcineurin. In T-cells responding to antigen presentation, an increase in intracellular Ca²⁺ activates calcineurin, which subsequently dephosphorylates the transcription factor called the nuclear factor of activated T-cells (“NFAT”). Dephosphorylated NFAT undergoes a molecular change, e.g., homodimerization that allows it to cross into the nucleus, and promotes the expression of T-cell activation genes. CsA and other immunosuppressive cyclosporin derivatives inhibit calcineurin which results in the inhibition of expression of cytokine genes, e.g., interleukin-2 (IL-2) that promotes T-cell activation and proliferation, i.e., immunosuppressive activity.

Human Immunodeficiency Viruses and Cyclosporin A or Non-Immunosuppressive Cyclosporins

Human immunodeficiency viruses (“HWs”) are lentiviruses, a family of mammalian retroviruses evolved to establish chronic persistent infection with gradual onset of clinical symptoms. There are two major families of HIV. Most of the epidemic involves HIV-1; HIV-2 is a close relative whose distribution is concentrated in western Africa.

Human cyclophilins A and B have been identified as cellular proteins which bind specifically to HIV-1 Gag polyprotein, p555^{gag}. Gag proteins play a major role in several steps of the virus life cycle, including the assembly and release of virions (Willis et al., “Form, Function, and Use of Retroviral Gag Proteins,” AIDS 5:639-654 (1991)). A cleavage product of the Gag polyprotein, the capsid protein, has been shown to bind specifically to cyclophilin A. Cyclophilin A is functionally associated with the HIV-1 virions through interaction with the Gag polyprotein. This interaction between cyclophilin A and Gag proteins is inhibited by the immunosuppressive drug, cyclosporin A (Thali et al., “Functional Association of Cyclophilin A With HIV-1 Virions,” Nature 372:363-365 (1994)).

Cyclosporin A has demonstrated in vitro-antiviral activity against HIV-1 (Karpas et al., “Inhibition of Human Immunodeficiency Virus and Growth of Infected T-cells by the Immunosuppressive Drugs Cyclosporin A and FK 506,” Proc. Natl. Acad. Sci. USA 89:8351-8355 (1992)); however, initial in vivo studies in which cyclosporin A was administered as a monotherapy in HIV-infected patients at advanced stages of disease did not show a beneficial effect from the treatment (Levy et al., “Long-Term Follow-Up of HIV Positive Asymptomatic Patients Having Received Cyclosporin A,” Adv. Ex. Med. Biol. 374:229-234 (1995)). U.S. Pat. No. 4,814,323 to Andrieu et al. reported that administration of cyclosporins may be used for the prevention of AIDS in patients infected with the virus before the appearance of the AIDS symptoms, that is patients with no symptoms or patients with AIDS related complex.

Highly active antiretroviral therapy (“HAART”) has dramatically decreased the HIV-related morbidity and mortality rates among HIV-infected patients and the transmission of HIV from mother to child by efficiently suppressing viral replication (Palella et al., “Declining Morbidity and Mortality Among Patients With Advanced Human Immunodeficiency Virus Infection,” N. Eng. J. Med. 338:853-860 (1998)). Limitations of HAART have become better understood. Thus, the virus can be suppressed to undetectable levels but not eradicated. In addition, there is an ever-growing list of side effects, the eventual development of resistance, and the cost and complexity of HAART regimens that must be contended with.

HAART covers a broad range of antiretroviral agents that include nucleoside reverse transcriptase inhibitors (“NRTI”), nonnucleoside reverse transcriptase inhibitors (“NNRTI”), HIV protease inhibitors, and fusion inhibitors. Specific examples of antiviral agents from each of these families include: Zidovudine, Didanosine, Stavudine, and Lamivudine from the NRTI antiviral class; Nevirapine, Efavirenz, and Delavirdine from the NNRTI antiviral class; Saquinovir, Indinavir, and Ritonavir from the HIV protease inhibitor class; and Enfuvirtide from the fusion inhibitor antiviral class.

From an immunological standpoint, the introduction of HAART allows for only a partial immune reconstitution. Indeed, ex vivo measures of immune function do not generally normalize and, most importantly, HIV-specific T cell responses remain almost invariably impaired. Though several variables have been identified that correlate with the degree of immune reconstitution during HAART, the actual underlying mechanism(s) responsible for such an incomplete immune reconstitution are still poorly understood and likely reflect the severe HIV-driven perturbations in T cell dynamics and homeostasis and the interaction between host and viral factors (Douek, “Disrupting T-Cell Homeostasis: How HIV-1 Infection Causes Disease,” AIDS Rev. 5:172-177 (2003)).

A strategy aimed at the broadest immune reconstitution, possibly overcoming the limitations of HAART, consists in the adjuvant use of immunomodulants. By combining cyclosporin A with HAART, the goal is to contain the immune activation, either virus-specific or owing to non-specific “by-stander” activation. Results from pilot studies in HIV-infected patients has shown that the rapid shutdown of T-cell activation induced by cyclosporin A has produced a more rapid and stable increase in CD4+ T-cells and a significant long-term increase in IFN-γ secreting CD4+ and CD4+ CCR7-T-cells, establishing a more favorable immunological set-point (Bandera et al., “Immunomodulants in HIV Infection,”Expert Opin. Ther. Patents 15(9): 1115-1131 (2005)). Determination of the long-term efficacy must be assessed in order to understand if this approach truly has value.

SDZ NIM 811 is a cyclosporin analogue that is completely devoid of immunosuppressive activity but exhibits potent and selective anti-HIV-1 activity (Mlynar et al., “The Non-Immunosuppressive Cyclosporin A Analogue SDZ NIM 811 Inhibits Cyclophilin A Incorporation Into Virions and Virus Replication in Human Immunodeficiency Virus Type-1-Infected Primary and Growth-Arrested Cells,” J. General Virology 78:825-835 (1997)). SDZ NIM 811 does not prevent the activation of CD4+ T-cell activation as cyclosporin A does. In a manner similar to cyclosporin A, it is proposed that SDZ NIM 811 interferes with the HIV-1 Gag-cyclophilin A interaction to effect its antiviral activity.

SDZ NIM 811 does not inhibit calcineurin and possesses none of the immunosuppressive activity of cyclosporin A. The potent inhibition of calcineurin by cyclosporin, in addition to being responsible for the potent immunosuppressive activity of cyclosporin A, is also believed to be the cause of the toxicity and the narrow therapeutic index of this drug. Separation of immunosuppressive and antiviral activity could lead to novel antiviral cyclosporins with fewer side effects and improved therapeutic index. Elucidation of structure activity relationships for cyclosporins permits the design of non-immunosuppressive cyclosporin derivatives that retain potent (cyclophilin A) PPIase activity to achieve this goal (Bartz et al., “Inhibition of Human Immunodeficiency Virus Replication by Non-Immunosuppressive Analogs of Cyclosporin A,” Proc. Natl. Acad. Sci. USA 92:5381-5385 (1995)). European Patent No. 484 281, U.S. Pat. No. 5,767,069, U.S. Pat. No. 5,948,884, and French Patent Nos. 2,757,520, 2,757,521, and 2,757,522 disclose non-immunosuppressive cyclosporins with antiviral activity.

Hepatitis C Virus and Cyclosporin A

Recently, cyclosporin A, the most widely prescribed immunosuppressive drug, was reported to be clinically effective against hepatitis C viral (HCV) infection (Nakagawa et al., “Specific Inhibition of Hepatitis C Virus Replication by Cyclosporin A,” Biochem. Biophys. Res. Commun. 313:42-47 (2004)). The authors of the Nakagawa et al. paper state that certain chaperone activities, such as those of cyclophilins, may be crucial for the processing and maturation of the viral proteins and for viral replication.

A subsequent controlled clinical trial showed that a combination of cyclosporin A with interferon α2b is more effective than interferon monotherapy, especially in patients with high viral loads (Inoue et al., “Combined Interferon α2b and Cyclosporin A in the Treatment of Chronic Hepatitis C: Controlled Trial,” J. Gastroenterol. 38:567-572 (2003)).

PCT International Patent Publication No. WO 2006/005610 recently described the use of a combination of cyclosporin A and pegylated interferon for treating hepatitis C viral infection. In addition, PCT International Patent Publication No. WO 2005/021028 relates to the use of non-immunosuppressive cyclosporins for treatment of HCV disorders. Also, Paeshuyse et al., “Potent and Selective Inhibition of Hepatitis C Virus Replication by the Non-Immunosuppressive Cyclosporin Analogue DEBIO-025,” Antiviral Research 65(3):A41 (2005) recently published results for a non-immunosuppressive cyclosporin analogue, DEBIO-025, that exhibited potent and selective inhibition of hepatitis C virus replication. Notably, the cyclosporin derivative DEBIO-025 is also effective for the treatment of HIV-1 (Rosenwirth et al., “Debio-025, A Novel Non-Immunosuppressive Cyclosporine Analog with Potent Anti-Human Immunodeficiency Virus Type 1 Activity: Pharmacological Properties and Mode of Action,” Antiviral Research 65(3):A42-A43 (2005)). Debio-025 does possess potent binding affinity for cyclophilin A.

There is still a large need for novel cyclosporin analogues that have therapeutic utility in the treatment of viral-induced diseases.

The present invention is directed to achieving these objectives.

### SUMMARY OF THE INVENTION

The present invention relates to a method of preventing or treating a mammal with a viral-induced disorder. The method involves administering to the mammal a therapeutically effective amount of a compound having the following formula: 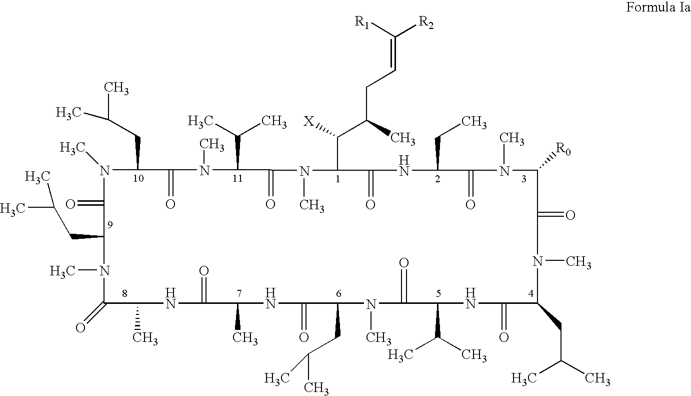
where:
X is OH or OAc;
R₀ is H or CH₂OR₃;
R₁ is H or D;
R₂ is selected from the group consisting of:
halogen,
C₁-C₆ halogenated saturated straight or branched carbon chain,
C₂-C₆ halogenated unsaturated straight or branched carbon chain,
C₃-C₆ substituted and unsubstituted cycloalkyl,
C₁-C₆ saturated straight or branched carbon chain containing amino group,
—CH═N—OR₄, and
—CH═N—NR₄R₅;
   R₃ is selected from the group consisting of:
hydrogen,
alkanoyl,
alkenoyl,
alkynoyl,
aryloyl,
arylalkanoyl,
alkylaminocarbonyl,
aryl aminocarbonyl,
arylalkylaminocarbonyl,
alkyloxycarbonyl,
aryloxycarbonyl, and
arylalkyloxycarbonyl;
   R₄ and R₅ are the same or different and independently selected from the group consisting of:
hydrogen,
C₁-C₆ saturated straight or branched carbon chain,
C₃-C₆ unsaturated straight or branched carbon chain,
C₃-C₆-substituted and unsubstituted cycloalkyl,
C₁-C₄ carbon chain containing an aryl or heteroaryl,
substituted and unsubstituted aryl,
substituted and unsubstituted heteroaryl,
alkanoyl,
alkenoyl,
alkynoyl,
aryloyl,
arylalkanoyl,
alkylaminocarbonyl,
arylaminocarbonyl,
arylalkylaminocarbonyl,
alkyloxycarbonyl,
aryloxycarbonyl,
arylalkyloxycarbonyl,
alkylsulfonyl, and
arylsulfonyl; and
   R₄ together with R₅ results in the formation of a cyclic moiety of C₂-C₆ optionally containing heteroatom or heteroatoms,
   wherein the compound is a cis geometric isomer, a trans geometric isomer, or a mixture of the cis and the trans geometric isomers or a pharmaceutically acceptable salt thereof,
   under conditions effective to prevent or treat the viral-induced disorder.

The present invention also relates to a method of preventing or treating a mammal with a viral-induced disorder. The method involves administering to the mammal a therapeutically effective amount of a compound having the following formula: 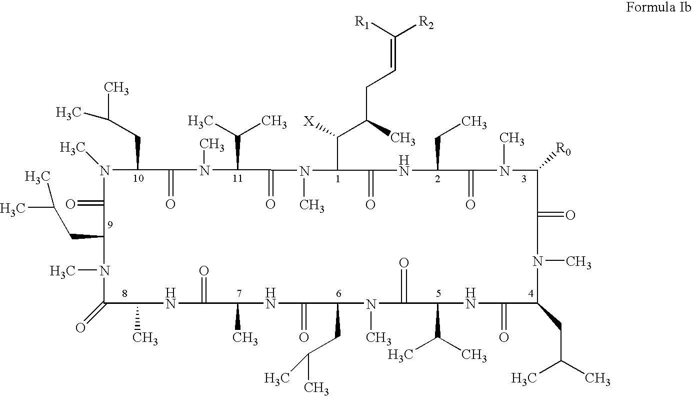
where:
X is OH or OAc;
R₀ is H or CH₂OR₃;
R₁ is halogen;
R₂ is selected from the group consisting of:
hydrogen,
deuterium,
halogen,
C₁-C₆ saturated straight or branched carbon chain, optionally containing halogen,
C₂-C₆ unsaturated straight or branched carbon chain, optionally containing halogen,
C₃-C₆ substituted and unsubstituted cycloalkyl,
substituted and unsubstituted aryl, and
substituted and unsubstituted heteroaryl; and
   R₃ is selected from the group consisting of:
hydrogen,
alkanoyl,
alkenoyl,
alkynoyl,
aryloyl,
arylalkanoyl,
alkylaminocarbonyl,
arylaminocarbonyl,
arylalkylaminocarbonyl,
alkyloxycarbonyl,
aryloxycarbonyl, and
arylalkyloxycarbonyl,
   wherein the compound is a cis geometric isomer, a trans geometric isomer, or a mixture of the cis and the trans geometric isomers or a pharmaceutically acceptable salt thereof,
   under conditions effective to prevent or treat the viral-induced disorder.

The present invention discloses novel cyclosporin derivatives that are chemically modified from cyclosporin A. In particular, the present invention discloses cyclosporin analogues containing a chemically modified side chain at the position one amino acid and optionally a substitution at the position three amino acid of cyclosporin A.

The present invention discloses novel cyclosporin analogues which are effective as antiviral agents. The cyclosporin derivatives of the present invention used to treat viral infections may possess potent immunosuppressive activity (via inhibition of calcineurin) or may be completely devoid of immunosuppressive activity (do not inhibit calcineurin). However, the mechanism that the immunosuppressive and non-immunosuppressive cyclosporin compounds share is their activity at cyclophilin A.

### BRIEF DESCRIPTION OF THE DRAWING

FIG. 1 depicts the results from a concanavalin A (ConA)-stimulated splenocyte assay, where the novel cyclosporin analogue compounds of the present invention (disclosed in Examples 9 and 11) are shown to possess enhanced potency in immunosuppression, compared to cyclosporin A.

### DETAILED DESCRIPTION OF THE INVENTION

The present invention relates to a method of preventing or treating a mammal with a viral-induced disorder. The method involves administering to the mammal a therapeutically effective amount of a compound having the following formula: 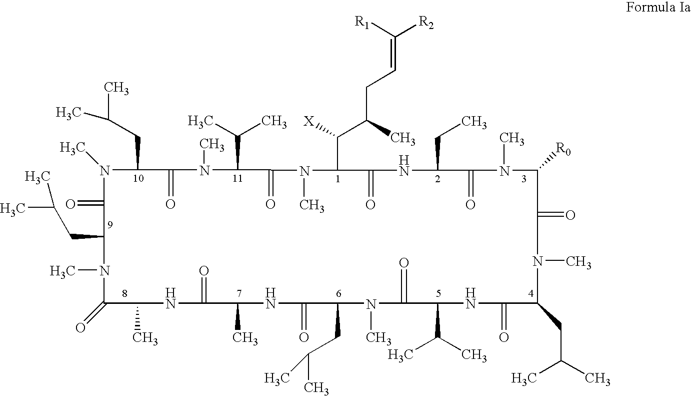
where:
X is OH or OAc;
R₀ is H or CH₂OR₃;
R₁ is H or D;
R₂ is selected from the group consisting of:
halogen,
C₁-C₆ halogenated saturated straight or branched carbon chain,
C₂-C₆ halogenated unsaturated straight or branched carbon chain,
C₃-C₆ substituted and unsubstituted cycloalkyl,
C₁-C₆ saturated straight or branched carbon chain containing amino group,
—CH═N—OR₄, and
—CH═N—NR₄R₅;
   R₃ is selected from the group consisting of:
hydrogen,
alkanoyl,
alkenoyl,
alkynoyl,
aryloyl,
arylalkanoyl,
alkylaminocarbonyl,
arylaminocarbonyl,
arylalkylaminocarbonyl,
alkyloxycarbonyl,
aryloxycarbonyl, and
arylalkyloxycarbonyl;
   R₄ and R₅ are the same or different and independently selected from the group consisting of:
hydrogen,
C₁-C₆ saturated straight or branched carbon chain,
C₃-C₆ unsaturated straight or branched carbon chain,
C₃-C₆-substituted and unsubstituted cycloalkyl,
C₁-C₄ carbon chain containing an aryl or heteroaryl,
substituted and unsubstituted aryl,
substituted and unsubstituted heteroaryl,
alkanoyl,
alkenoyl,
alkynoyl,
aryloyl,
arylalkanoyl,
alkylaminocarbonyl,
arylaminocarbonyl,
arylalkylaminocarbonyl,
alkyloxycarbonyl,
aryloxycarbonyl,
arylalkyloxycarbonyl,
alkylsulfonyl, and
arylsulfonyl; and
   R₄ together with R₅ results in the formation of a cyclic moiety of C₂-C₆ optionally containing heteroatom or heteroatoms,
   wherein the compound is a cis geometric isomer, a trans geometric isomer, or a mixture of the cis and the trans geometric isomers or a pharmaceutically acceptable salt thereof,
   under conditions effective to prevent or treat the viral-induced disorder.

One embodiment of the present invention relates to the above compound of Formula Ia, where: X=OH or OAc; R₀=H, CH₂OH, or CH₂OAc; R₁=H or D; and R₂═F, Cl, Br, or I.

Another embodiment of the present invention relates to the above compound of Formula Ia, where: X=OH or OAc; R₀=H, CH₂OH, or CH₂OAc; R₁=H or D; and R₂=CF₃, CH₂F, or CH₂Cl.

Another embodiment of the present invention relates to the above compound of Formula Ia, where: X=OH or OAc; R₀=H, CH₂OH, or CH₂OAc; R₁=H or D; and R₂=—CH═CHF, —CH═CHCl, —CH═CHBr, or —CH═CHI.

Another embodiment of the present invention relates to the above compound of Formula Ia, where: X=OH or OAc; R₀=H, CH₂OH, or CH₂OAc; R₁=H or D; and R₂=—CH═CH—C≡CH, —CH═CH—C≡C—CH₃, or —CH═CH—C≡C—CH═CH₂.

Another embodiment of the present invention relates to the above compound of Formula Ia, where: X=OH or OAc; R₀=H, CH₂OH, or CH₂OAc; R₁=H or D; and R₂ is cyclopropyl.

Another embodiment of the present invention relates to the above compound of Formula Ia, where: X=OH or OAc; R₀=H, CH₂OH, or CH₂OAc; R₁=H or D; and R₂=—CH═N—OH, —CH═N—OCH₃, —CH═N—OCH₂CH₃, —CH═N—NHCH₃, or —CH═N—N(CH₃)₂.

Another embodiment of the present invention relates to the above compound of Formula Ia, where:

X=OH or OAc,

R₀=H,

R₁=H or D, and

R₂=Cl, Br, I, CF₃, C₃F₇, C₄F₉, CH₂F, CH₂Cl, -cyclopropyl, —CH═CHCl, —CH═CHBr, —CH═CHI, —CH═CHCF₃, —C(CF₃)═CH₂, —C≡CC₄H₉, —CH═CH—C≡CH, —CH═CH—C≡CCH₃, —CH═CH—C≡CSi(CH₃)₃, —CH═CH—C≡C—CH═CH₂, —CH═CH—C≡C—CH(OH)CH₃, —CH₂NHCH₃, —CH₂N(CH₃)₂, —CH₂N(CH₃)(Ac), —CH₂-pyrrolidine, —CH₂-piperidine, —CH₂-morphorline, —CH₂-thiomopholine, —CH₂-methylpiperizine, —CH═N—OH, —CH═N—OCH₃, —CH═N—OCH₂CH₃, —CH═N—OCH₂CH═CH₂, —CH═N—OCH₂Ph, —CH═N—N(CH₃)₂, —CH═N—NHCH₃, or —CH═N—NHSO₂C₆H₄CH₃.

Another embodiment of the present invention relates to the above compound of Formula Ia, where:

X=OH or OAc,

R₀=CH₂OH or CH₂OAc,

R₁=H, and

R₂=Cl, Br, I, CF₃, CH₂F, Ph, —CH═CHCl, —CH═CHBr, —CH═CHI, —CH═CH₂, or —CH═CD₂.

The present invention also relates to a method of preventing or treating a mammal with a viral-induced disorder. The method involves administering to the mammal a therapeutically effective amount of a compound having the following formula: 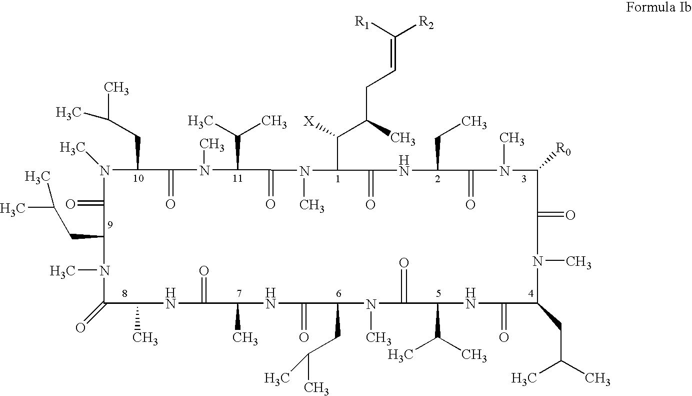
where:
X is OH or OAc;
R₀ is H or CH₂OR₃;
R₁ is halogen;
R₂ is selected from the group consisting of:
hydrogen,
deuterium,
halogen,
C₁-C₆ saturated straight or branched carbon chain, optionally containing halogen,
C₂-C₆ unsaturated straight or branched carbon chain, optionally containing halogen,
C₃-C₆ substituted and unsubstituted cycloalkyl,
substituted and unsubstituted aryl, and
substituted and unsubstituted heteroaryl; and
   R₃ is selected from the group consisting of:
hydrogen,
alkanoyl,
alkenoyl,
alkynoyl,
aryloyl,
arylalkanoyl,
alkylaminocarbonyl,
arylaminocarbonyl,
arylalkylaminocarbonyl,
alkyloxycarbonyl,
aryloxycarbonyl, and
arylalkyloxycarbonyl,
   wherein the compound is a cis geometric isomer, a trans geometric isomer, or a mixture of the cis and the trans geometric isomers or a pharmaceutically acceptable salt thereof,
   under conditions effective to prevent or treat the viral-induced disorder.

Another embodiment of the present invention relates to the compound of Formula Ib, where:

X=OH or OAc,

R₀=H,

R₁=Cl, and

R₂=H, D, C₁, CF₃, or Ph.

Another embodiment of the present invention relates to the compound of Formula Ib, where:

X=OH or OAc,

R₀=H,

R₁=Br or I, and

R₂=H, D, or CH₃.

In particular, the present invention relates to novel halogenated cyclosporin analogues, including cyclosporin vinyl halides and allylic halides.

The present invention also discloses methods for preparation of novel cyclosporin analogue compounds represented by Formula Ia and Formula Ib and their utility as pharmaceutical agents for treatment of various diseases. The present invention also describes the utility of halogenated cyclosporin analogues (vinyl halides and allylic halides) as synthetic intermediates that can be transformed into additional novel cyclosporin derivatives.

The starting material for the preparation of the compounds of the present invention is cyclosporin A. The structure of cyclosporin A, a cycloundecapeptide, and the position numbering for each amino acid in the ring is shown below:

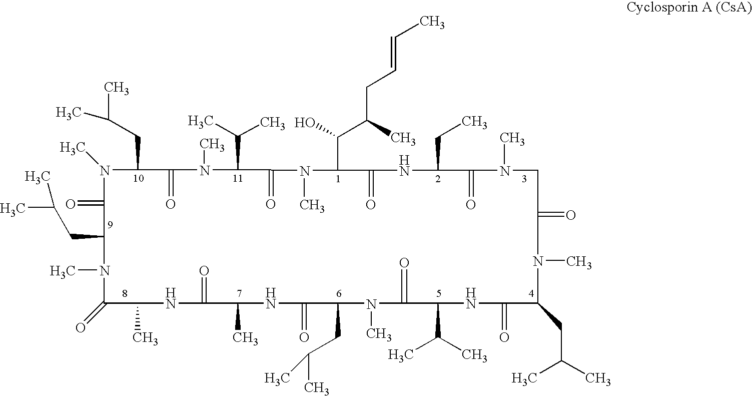

Cyclosporin A can also be represented by Formula Ia, as shown below:

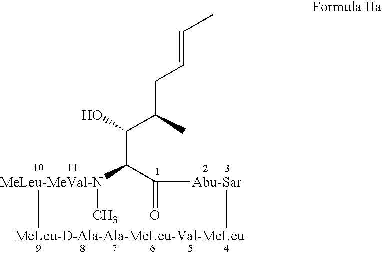

The novel cyclosporin analogues of the present invention are derived from cyclosporin A or a key intermediate prepared by modification at the position three amino acid of cyclosporin A. As shown in Scheme 1, such a key intermediate (Formula IIb) can be prepared by deprotonation of cyclosporin A with lithium diisopropylamide (LDA), followed by treatment with formaldehyde (Seebach et al, “Modification of Cyclosporin A: Generation of an Enolate at the Sarcosine Residue and Reaction With Electrophiles,” Helv. Chim. Acta, 76:1564-1590 (1993), which is hereby incorporated by reference in its entirety).

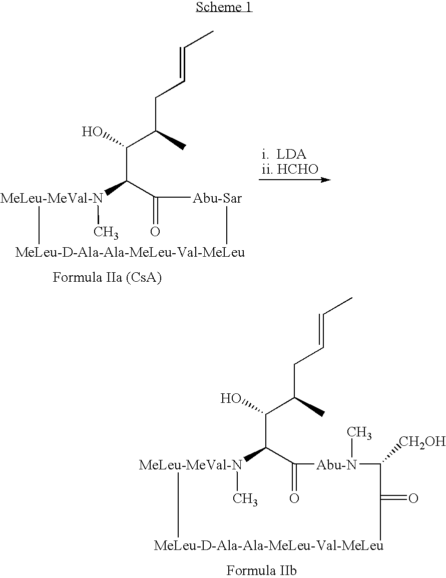

According to one embodiment of the present invention, novel cyclosporin vinyl halides can be prepared by employing Takai reaction as a key step, as outlined in Scheme 2. Acetylation of cyclosporin A (Formula Ia) or cyclosporin diol intermediate of Formula IIb with acetic anhydride, followed by oxidative cleavage of the double bond with ozone, generates cyclosporin aldehyde of Formula III smoothly. Treatment of the cyclosporin aldehyde with haloform-CrCl₂ complex affords novel cyclosporin vinyl halides of Formula Ia (Takai et al, “Simple and Selective Method for RCHO→(E)-RCH═CHX Conversion by Means of a CHX₃—CrCl₂ System,” J. Am. Chem. Soc., 108:7408-7410 (1986), which is hereby incorporated by reference in its entirety). Various haloforms, such as chloroform, bromoform, and iodoform can be applied. Usage of excess haloform and CrCl₂ seems to be necessary to obtain the desired vinyl halide in good to excellent yield (50-80%). This stereoselective chemistry provided a halogenated olefin of Formula Ia in exclusively the trans-configuration (R₁=H or D; R₂=halogen). The acetyl protection group(s) can be removed by treatment with potassium carbonate in methanol (see Scheme 2).

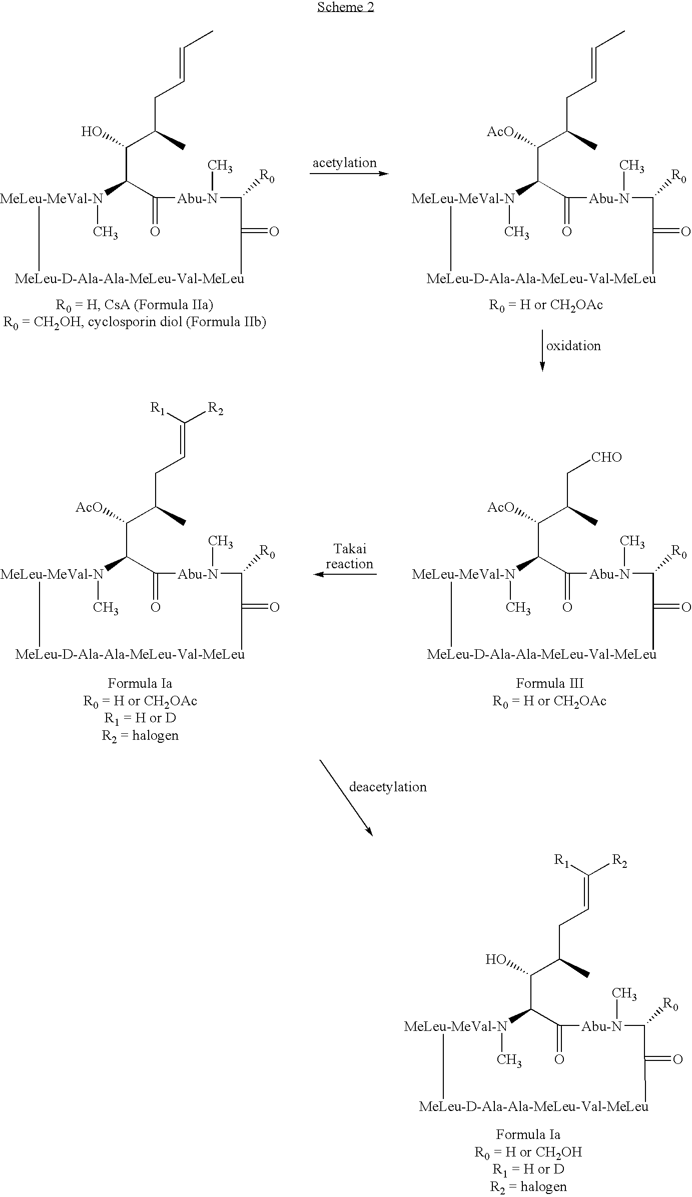

The novel cyclosporin vinyl halides of Formula Ib in the present invention can be prepared via an alternative approach by application of phosphorous ylide chemistry (Wittig reaction, Horner-Emmons reaction, or other modified Wittig conditions), as shown in Scheme 3. This chemistry converts the cyclosporin aldehyde of Formula III to the halogenated olefin of Formula Ib effectively. The reaction generates either the cis-isomer of the olefin or a separable mixture of cis- and trans-isomers. Typically, the phosphorous ylide species under Wittig, Horner-Emmons, or other modified Wittig conditions are generated by treatment of various phosphonium salts or phosphonates with a strong base, such as n-butyllithium or sodium bis(trimethylsilyl)amide. The deacetylation is conducted under the same conditions as described in Scheme 2.

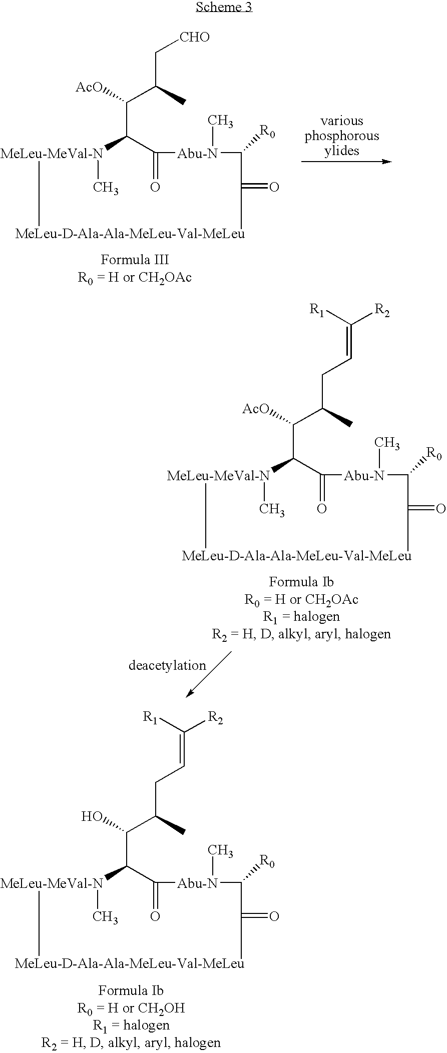

Utilizing the same strategy described in Scheme 2, halogenated cyclosporin diene can be prepared via a Takai reaction with α,β-unsaturated aldehyde of Formula IV, which is generated by application of olefin cross metathesis on cyclosporin (Scheme 4). In the last decade, ruthenium catalyzed olefin metathesis has emerged as a powerful synthetic tool for the formation of carbon-carbon bonds (Chatterjee et al, “A General Model for Selectivity in Olefin Cross Metathesis,” J. Am. Chem. Soc., 125:11360-11370 (2003); Connon et al, “Recent Development in Olefin Cross Metathesis,” Angew. Chem. Int. Ed., 42:1900-1923 (2003), which are hereby incorporated by reference in their entirety). There are three main variations on olefin metathesis: (a) cross metathesis; (b) ring opening/close metathesis; and (c) intermolecular enyne metathesis. As an acyclic carbon-carbon bond-forming method, olefin cross metathesis has numerous advantages: (1) the process is catalytic (typically 1-5 mol % of catalyst required); (2) high yield can be obtained under mild conditions in a relatively short reaction time; (3) a wide range of functional groups are tolerated, with minimal substrate protection necessary; and (4) the reaction is relatively atom-economic, and gaseous ethylene is usually the only byproduct, which is an important consideration in industrial applications (Connon et al, “Recent Development in Olefin Cross Metathesis,” Angew. Chem. Int. Ed., 42:1900-1923 (2003), which is hereby incorporated by reference in its entirety).

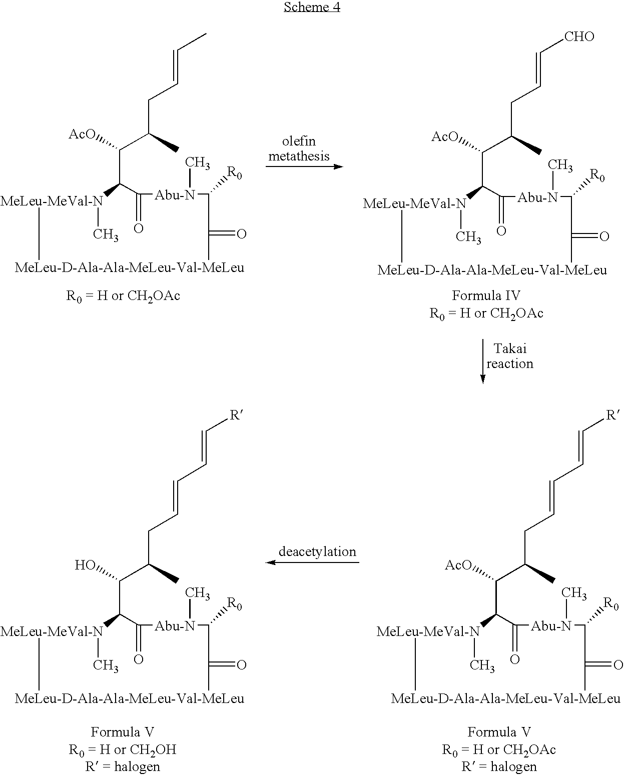

As shown in Scheme 4, olefin cross metathesis of acetyl-protected cyclosporin A or cyclosporin diol is carried out with acrolein acetals (such as acrolein dimethyl acetal and 2-vinyl-1,3-dioxolane) in the presence of Grubbs' catalyst in methylene chloride or toluene. The reaction provides an acetal intermediate which is hydrolyzed during purification by high pressure liquid chromatography, using acetonitrile-water-trifluoroacetic acid as a solvent system to afford trans-α,β-unsaturated aldehyde of Formula IV directly in good to excellent yield (60-80%). The catalyst can be either Grubbs' catalyst 2^{nd} generation (Schwab et al, “A Series of Well-Defined Metathesis Catalysts-Synthesis of [RuCl₂(═CHR′)(PR₃)₂] and Its Reactions,” Angew. Chem. Int. Ed., 34:2039-2041 (1995), which is hereby incorporated by reference in its entirety) or Hoveyda-Grubbs catalyst (Scholl et al, “Synthesis and Activity of a New Generation of Ruthenium-Based Olefin Metathesis Catalysts Coordinated with 1,3-Dimesityl-4,5-dihydroimidazol-2-ylidene Ligands,” Org. Lett., 1:953 (1999); Sanford et al, “Mechanism and Activity of Ruthenium Olefin Metathesis Catalysts,” J. Am. Chem. Soc., 123:6543-6554 (2001), which are hereby incorporated by reference in their entirety). This stereoselective chemistry provides an α,β-unsaturated aldehyde of Formula IV exclusively in the trans-geometric isomer.

Treatment of α,β-unsaturated aldehyde of Formula IV with haloform and CrCl₂ in tetrahydrofuran provides a halogenated cyclosporin diene of Formula V as a trans-isomer. Only a small amount of cis-isomer is observed under these conditions. Finally, acetyl protection group(s) can be removed with potassium carbonate in methanol (Scheme 4).

According to another embodiment of the present invention, cyclosporin vinyl halides can be used as powerful synthetic intermediates for palladium or nickel-catalyzed couplings (such as Stille coupling, Suzuki coupling, Negishi coupling, and Sonogashira coupling) to build a new carbon-carbon bond. As shown in Scheme 5, Stille coupling of the cyclosporin vinyl iodide of Formula VI with organotin reagents, in the presence of Pd(CH₃CN)₂Cl₂, affords a novel cyclosporin cyclopropyl derivative and a diene analogue respectively, while Sonogashira coupling with alkyne provides enyne analogue. Similar reactions can be performed on the halogenated diene of Formula V with organotin reagents, organozinc reagents, boronic acids, or alkynes to prepare novel cyclosporin analogues.

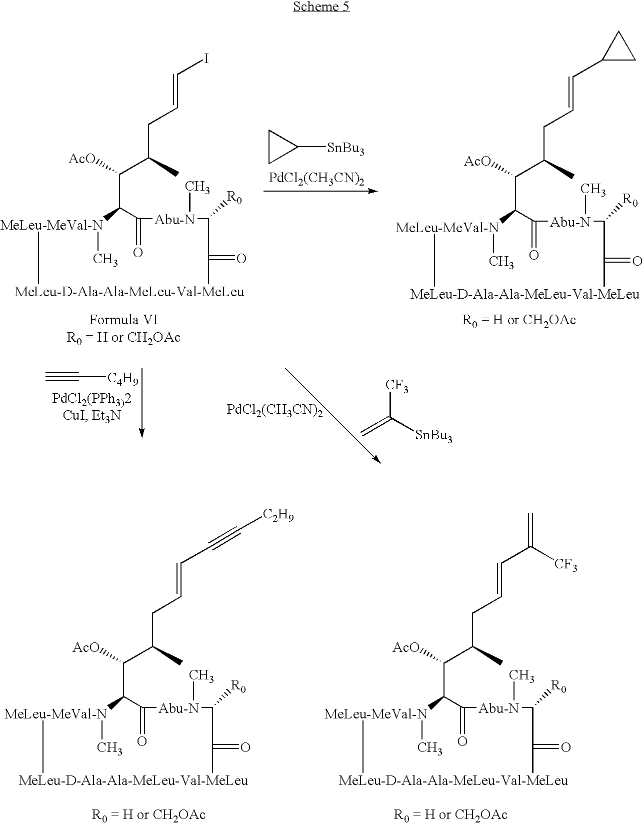

According to another embodiment of the present invention, cyclosporin allylic halides can be prepared via olefin cross metathesis with a Grubbs catalyst, as shown in Scheme 6. Utilizing an allylic chloride of Formula VII as a key intermediate, various cyclosporin amine derivatives can be obtained.

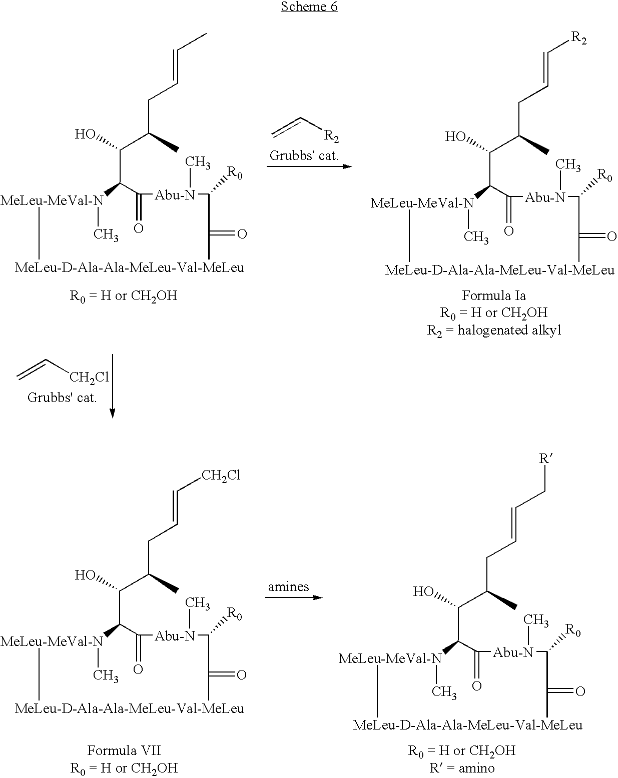

Another embodiment of the present invention relates to a so-called “soft drug” strategy (Lazarova et al., “Synthesis a n d Biological Evaluation of Novel Cyclosporin A Analogues: Potential Soft Drugs for the Treatment of Autoimmune Diseases,” Journal of Medicinal Chemistry, 46:674-676 (2003); Little et al., “Soft Drugs Based on Hydrocortisone: The Inactive Metabolite Approach and Its Application to Steroidal Anti-inflammatory Agents,” Pharm. Res., 16:961-967 (11999), which are hereby incorporated by reference in their entirety). Incorporation of a C—N bond leads to the preparation of α-unsaturated oximes (C═N—OR) and hydrazones (C═N—NR₂) of Formula VIII. The active α,β-unsaturated oximes (C═N—OR) and hydrazones (C═N—NR₂) of the present invention can be hydrolyzed and inactivated under physiological conditions. Therefore, the C═N moiety of the novel cyclosporin analogues of Formula VIII provides a simple means to control the hydrolytic half-life of the soft drug, thus minimizing system exposure and toxicity. As shown in Scheme 7, the treatment of α,β-unsaturated aldehyde of Formula IV with hydroxylamines or alkyloxyamines (RONH₂) and hydrazines (R₂NNH₂) affords the corresponding α,β-unsaturated oximes (C═N—OR) and hydrazones (C═N—NR₂) of Formula VIII, respectively.

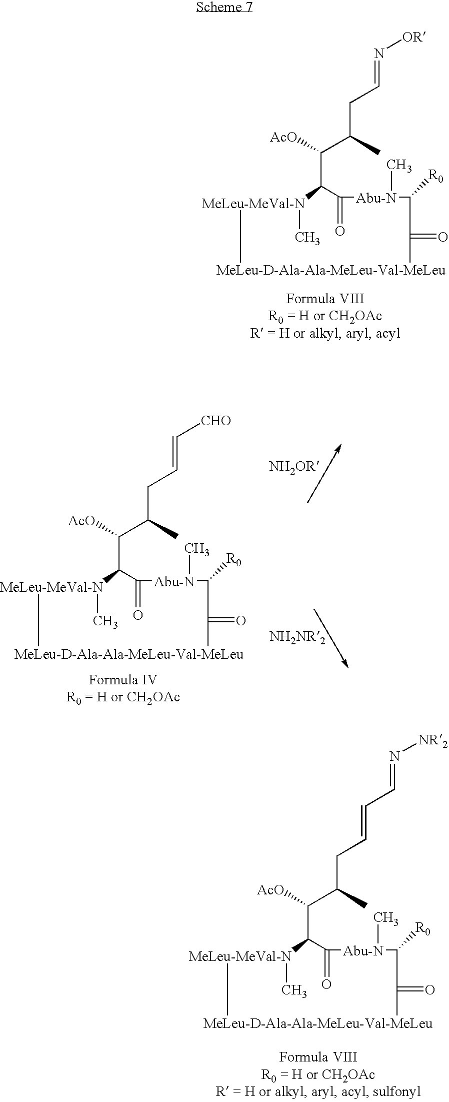

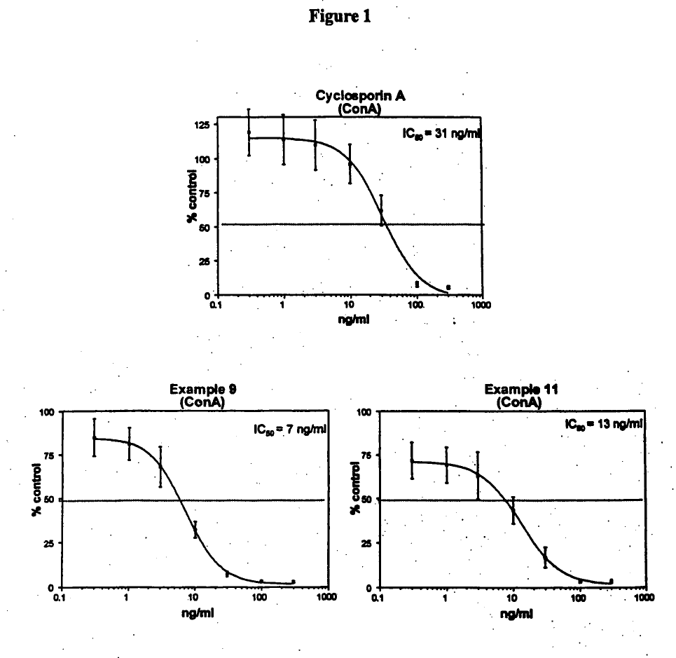

Some of the compounds disclosed in the present invention are useful as immunosuppressive agents. Administration of these compounds suppresses the immune response in organ transplant patients and, thus, prevents allograft rejection. The compounds of the present invention may possess immunosuppressive activity similar to or more potent than cyclosporin A. For example, as shown in FIG. 1, the novel cyclosporin analogue compounds disclosed in Examples 9 and 11 possess enhanced potency in immunosuppresion in the concanavalin A stimulated splenocyte assay, compared to cyclosporin A. Table 1 shows the immunosuppressive activities of several novel cyclosporin analogue compounds disclosed in the present application. (The third column in Table 1 contains cyclosporin A positive control values included for comparison.)

TABLE 1
Immunosuppressive Activities of Novel Cyclosporin
Analogue Compounds of the Present Invention

| Example where the | | |
|---|---|---|
| Novel Cyclosporin | | |
| Analogue Compound | | |
| is Disclosed | IC₅₀ (ng/mL) | IC₅₀ (ng/mL) of CsA |
| Example 5 | 22 | 25 |
| Example 6 | 11 | 25 |
| Example 9 | 7 | 31 |
| Example 11 | 13 | 31 |
| Example 19 | 9 | 6 |
| Example 26 | 5 | 9 |
| Example 31 | 38 | 8 |
| Example 34 | 12 | 5 |
| Example 56 | 15 | 31 |
| Example 58 | 42 | 31 |
| Example 67 | 7 | 6 |
| Example 69 | 4 | 9 |
| Example 71 | 8 | 6 |
| Example 72 | 5 | 6 |

The compounds disclosed in the present invention are useful for the prevention or treatment of viral-induced disorders that are dependent upon the presence of cyclophilin A. The compounds of the present invention used to treat these viral infections may possess potent immunosuppressive activity (via inhibition of calcineurin) or may be completely devoid of immunosuppressive activity (do not inhibit calcineurin). However, the mechanism that the immunosuppressive and non-immunosuppressive cyclosporin compounds share is their activity at cyclophilin A.

Cyclophilin A enzyme activity, i.e., peptidyl-prolyl cis-trans isomerase activity, is important to the folding and trafficking of proteins. The HIV infectivity of CD4+ T-cells and viral replication are dependent upon the incorporation of cyclophilin A into HIV-1 virions through interactions with the Gag polyprotein. Inhibition of the cyclophilin A enzyme activity is necessary and sufficient for anti-HIV-1 activity.

In one embodiment of the present invention, the viral-induced disorder is a human immunodeficiency virus (HIV)-induced disorder. Thus, compounds of the present invention that lack immunosuppressant activity as determined by the Concanavalin A (Con A)-stimulated murine splenocyte assay but retain potent peptidyl prolyl isomerase (PPIase) inhibitory (cyclophilin A) activity may possess anti-HIV activity. In addition, compounds of the present invention that have immunosuppressive activity as determined by the Con A-stimulated murine, splenocyte assay and also possess potent PPIase inhibitory (cyclophilin A) activity may possess anti-HIV activity.

In vitro biological assays that allow the determination of binding affinity to cyclophilin A or allow the determination of inhibition of peptidyl cis-trans isomerase activity are described in Handschumacher et al., “Cyclophilin: A Specific Cytosolic Binding Protein for Cyclosporin A,” Science 226:544-547 (1984) and Kofron et al., “Determination of Kinetic Constants for Peptidyl Prolyl cis-Trans Isomerases by an Improved Spectrophotometric Assay,” Biochemistry 30:6127-6134 (1991), respectively, which are both hereby incorporated by reference in their entirety.

The in vitro anti-HIV activity of compounds of the present invention can be measured in established cell line cultures as described by Mayaux et al., “Triterpene Derivatives That Block Entry of Human Immunodeficiency Virus Type 1 Into Cells,” Proc. Natl. Acad. Sci. USA 91:3564-3568 (1994), which is hereby incorporated by reference in its entirety.

In another embodiment of the present invention, the compound of the present invention is administered in combination with antiretroviral agents, such as nucleoside reverse transcriptase inhibitors, nonnucleoside reverse transcriptase inhibitors, human immunodeficiency virus protease inhibitors, fusion inhibitors, and combinations thereof. Examples of nucleoside reverse transcriptase inhibitors include, but are not limited to, Zidovudine, Didanosine, Stavudine, and Lamivudine. Examples of nonnucleoside reverse transcriptase inhibitors include, but are not limited to, Nevirapine, Efavirenz, and Delavirdine. Examples of human immunodeficiency virus protease inhibitors include, but are not limited to, Saquinovir, Indinavir, and Ritonavir. Examples of fusion inhibitors include, but are not limited to, Enfuvirtide.

Although cyclophilin PPIase activity would appear to be implicated in anti-HCV activity as it is for anti-HIV-1 activity, the hepatitis C virus (HCV) proteins that may interact with cyclophilin A have yet to be identified. In another embodiment of the present invention, the viral-induced disorder is a HCV-induced disorder. Hepatitis C infections or HCV induced disorders are, for example, chronic hepatitis, liver cirrhosis, or liver cancer (e.g., hepatocellular carcinoma). Thus, compounds of the present invention that lack immunosuppressant activity as determined by the Concanavalin A (Con A)-stimulated murine splenocyte assay but retain potent peptidyl prolyl isomerase (PPIase) inhibitory (cyclophilin A) activity may possess anti-HCV activity. In addition, compounds of the present invention that have immunosuppressive activity as determined by the Con A-stimulated murine splenocyte assay and also possess potent PPIase inhibitory (cyclophilin A) activity may possess anti-HCV activity. The compounds of the present invention may also be used as a prophylactic treatment for neonates born to HCV-infected mothers, for healthcare workers exposed to the virus, or for transplant recipients, e.g., organ or tissue transplant (e.g. liver transplant) recipients, to eliminate possible recurrent infection after transplantation.

In another embodiment of the present invention, the compound of the present invention is administered in combination with an interferon. Examples of interferons include, but are not limited to, interferon α2a and interferon α2b. The interferon can be a pegylated interferon. Examples of interferons include, but are not limited to, pegylated interferon α2a or pegylated interferon α2b.

Utility of the immunosuppressive or non-immunosuppressive cyclosporin compounds of the present invention in treating diseases or conditions from HCV infection can be demonstrated in standard animal or clinical tests in accordance with the methods described in Examples 89 and 90, for example.

Some of the compounds disclosed in the present invention also possess utility in the treatment of autoimmune and chronic inflammatory diseases such as asthma, rheumatoid arthritis, multiple sclerosis, psoriasis, and ulcerative colitis, to name only a few.

The compounds disclosed in the present invention are also useful for the treatment of ocular allergy and dry eye. Allergan is currently marketing a topical formulation of cyclosporin A called Restasis™ (cyclosporin ophthalmic emulsion) for the treatment of keratoconjunctivitis sicca or chronic dry eye syndrome in patients whose tear production is presumed to be suppressed due to ocular inflammation. While the exact mechanism of Restasis™ is unknown, it is thought to act as an immunomodulator with anti-inflammatory effects (“Annual Update 2003: Ophthalmic Drugs” Drugs of the Future, 28(3): 287-307 (2003); Clark et al., “Ophthalmic Drug Discovery,” Nature Reviews in Drug Discovery, 2:448-459 (2003), which are hereby incorporated by reference in their entirety).

For treatment of the above-mentioned diseases, therapeutically effective doses of the compounds of the present invention may be administered orally, topically, parenterally, by inhalation spray, or rectally in dosage unit formulations containing conventional non-toxic pharmaceutically acceptable carriers, adjuvants, and vehicles. The term parenteral, as used herein, includes subcutaneous injections, intravenous, intramuscular, intrasternal injection, or infusion techniques.

The pharmaceutical compositions containing the active ingredient may be in the form suitable for oral use, for example, as tablets, troches, lozenges, aqueous or oily suspensions, dispersible powders or granules, emulsions, hard or soft capsules, or syrups or elixirs. The pharmaceutical compositions of the present invention contain the active ingredient formulated with one or more pharmaceutically acceptable carriers. As used herein, the term “pharmaceutical acceptable carrier” means a non-toxic, inert solid, semi-solid or liquid filler, diluent, encapsulating material, or formulation auxiliary of any type. Some examples of pharmaceutically acceptable carriers are sugars such as lactose, glucose, and sucrose; starches such as corn starch or potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; phosphate buffer solutions; non-toxic, compatible lubricants such as sodium lauryl sulfate and magnesium stearate; as well as coloring agents, releasing agents, sweetening, and flavoring and perfuming agents. Preservatives and antioxidants, such as ethyl or n-propyl p-hydroxybenzoate, can also be included in the pharmaceutical compositions.

Dosage forms for topical or transdermal administration of the compounds disclosed in the present invention include ointments, pastes, creams, lotions, gels, plasters, cataplasms, powders, solutions, sprays, inhalants, or patches. The active component is admixed under sterile conditions with a pharmaceutically acceptable carrier and any needed preservatives or buffers, as may be required. The ointments, pastes, creams and gels may contain, in addition to an active compound of the present invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

For nasal administration, the compounds disclosed in the present invention can be administered, as suitable, in liquid or powdered form from a nasal applicator. Forms suitable for ophthalmic use will include lotions, tinctures, gels, ointment and ophthalmic inserts, as known in the art. For rectal administration (topical therapy of the colon), compounds of the present invention may be administered in suppository or enema form, in solution in particular, for example in vegetable oil or in an oily system for use as a retention enema.

The compounds disclosed in the present invention may be delivered to the lungs by the inhaled route either in nebulizer form or as a dry powder. The advantage of the inhaled route, over the systemic route, in the treatment of asthma and other diseases of airflow obstruction and/or chronic sinusitis, is that patients are exposed to very small quantities of the drug and the compound is delivered directly to the site of action.

Dosages of the compounds of the present invention employed for the treatment of the maladies identified in the present invention will vary depending on the site of treatment, the particular condition to be treated, the severity of the condition, the subject to be treated (who may vary in body weight, age, general health, sex, and other factors), as well as the effect desired.

Dosage levels ranging from about 0.05 mg to about 50 mg per kilogram of body weight per day are useful for the treatment of the conditions or diseases identified in the present invention. This means the amount of the compound disclosed in the present invention that is administered will range from 2.5 mg to about 2.5 gm per patient per day.

The amount of active ingredient that may be combined with the pharmaceutical carrier materials to produce a single dosage form will vary depending upon the host treated and the particular mode of administration. For example, a formulation intended for the oral administration of humans may contain from 2.5 mg to 2.5 gm of active compound of the present invention compounded with an appropriate and convenient amount of carrier material which may vary from about 5 to 95 percent of the total composition. Dosage unit forms will generally contain between from about 5 mg to about 500 mg of active compound of the present invention. Dosage for topical preparation will, in general be less (one tenth to one hundredth) of the dose required for an oral preparation.

### EXAMPLES

The following examples are provided to illustrate embodiments of the present invention but are by no means intended to limit its scope.

### Example 1

### Preparation of Cyclosporin Acetate

A solution of cyclosporin A (5.0 g, 4.16 mmol), acetic anhydride (7.80 mL, 83.2 mmol), and DMAP (760 mg, 6.2 mmol) in methylene chloride (40 mL) was stirred overnight at room temperature under a N₂ atmosphere. Saturated sodium bicarbonate solution (200 mL) was added to the solution and stirred for an additional 2 h. The mixture was extracted with ether, washed with 1 N HCl, neutralized with saturated sodium bicarbonate solution, washed with brine, dried over sodium sulfate, and concentrated in vacuo to afford cyclosporin acetate (4.92 g, 95%) as a white solid: ¹H NMR (300 MHz, CDCl₃) δ 8.57 (d, J=9.6 Hz, 1H), 8.04 (d, J=6.9 Hz, 1H), 7.51 (d, J=9.4 Hz, 1H), 7.47 (d, J=7.8 Hz, 1H), 5.67 (dd, J=11.0, 4.0 Hz, 1H), 5.60-5.44 (m, 2H), 5.39 (dd, J=11.7, 3.7 Hz, 1H), 5.32-5.13 (m, 4H), 5.06-4.93 (m, 2H), 4.85 (t, J=7.2 Hz, 1H), 4.77 (t, J=9.6 Hz, 1H), 4.65 (d, J=13.7 Hz, 1H), 4.41 (t, J=7.0 Hz, 1H), 3.46 (s, 3H), 3.26 (s, 3H), 3.24 (s, 3H), 3.21 (s, 3H), 3.10 (s, 3H), 2.68 (s, 3H), 2.66 (s, 3H), 2.50-2.35 (m, 1H), 2.25-1.80 (m, 6H), 2.08 (s, 3H), 2.01 (s, 3H), 1.75-1.55 (m, 6H), 1.45-0.75 (m, 55H); ESI MS m/z 1245 [C₆₄H₁₁₃N₁₁O₁₃+H]⁺.

### Example 2

### Preparation of Acetyl Cyclosporin Aldehyde

Ozone was bubbled into a solution of cyclosporin acetate from Example 1 (3.0 g, 2.4 mmol) in methylene chloride (70 mL) at −78° C. until a blue color was developed. The mixture was degassed with nitrogen for a few minutes and dimethylsulfide (3 mL) was added at −78° C. The reaction mixture was allowed to warm to room temperature and stirred for 3 h. The reaction mixture was concentrated in vacuo and the residue was dissolved in ethyl acetate (300 mL), washed with water (2×70 mL) and brine (70 mL), dried over sodium sulfate, filtered, and concentrated in vacuo to afford acetyl cyclosporin aldehyde (2.79 g, 94%) as a white solid. The crude was carried to the next step without further purification: ¹H NMR (300 MHz, CDCl₃) δ 9.60 (d, J=3.5 Hz, 1H), 8.55 (d, J=9.7 Hz, 1H), 7.96 (d, J=6.8 Hz, 1H), 7.52 (d, J=7.7 Hz, 1H), 7.46 (d, J=9.0 Hz, 1H), 5.67 (dd, J=11.0, 3.8 Hz, 1H), 5.60-5.45 (m, 2H), 5.32 (dd, J=12.1, 3.3 Hz, 1H), 5.24-5.10 (m, 2H), 5.08-4.90 (m, 2H), 4.84 (t, J=7.1 Hz, 1H), 4.73 (t, J=9.6 Hz, 1H), 4.64 (d, J=13.8 Hz, 1H), 4.41 (t, J=7.0 Hz, 1H), 3.46 (s, 3H), 3.29 (s, 6H), 3.21 (s, 3H), 3.08 (s, 3H), 2.67 (s, 3H), 2.65 (s, 3H), 2.50-2.35 (m, 2H), 2.25-1.80 (m, 6H), 1.99 (s, 3H), 1.75-1.55 (m, 3H), 1.50-0.75 (m, 57H); ESI MS m/z 1233 [C₆₂H₁₀₉N₁₁O₁₄+H]⁺.

### Example 3

### Preparation of Acetyl Cyclosporin Vinyl Chloride

Anhydrous CrCl₂ (100 mg, 0.81 mmol) was suspended in THF (3 mL) under an argon atmosphere and, then, a solution of acetyl cyclosporin aldehyde from Example 2 (100 mg, 0.081 mmol) and CHCl₃ (29 mg, 0.243 mmol) in THF (1 mL) were added. The mixture was stirred at 40° C. under argon for 64 h. After cooling down to room temperature, the solvent was removed in vacuo and the residue was purified via semi-preparative HPLC to give the desired acetyl cyclosporin vinyl chloride (25 mg, 24%) as a white solid: ¹H NMR (CDCl₃, 500 MHz) δ 8.46 (d, J=9.3 Hz, 1H), 8.00 (d, J=6.9 Hz, 1H), 7.64 (d, J=9.0 Hz, 1H), 7.58 (d, J=7.8 Hz, 1H), 5.83 (m, 1H), 5.68 (d, J=7.2 Hz, 1H), 5.56 (d, J=1.1 Hz, 2H), 5.44 (d, J=13.5, 3.8 Hz, 1H), 5.22 (m, 1H), 5.06-4.94 (m, 3H), 4.85 (t, J=7.2 Hz, 1H), 4.77 (d, J=10.6 Hz, 1H), 4.64 (d, J=13.5 Hz, 1H), 4.43 (t, J=6.6 Hz, 1H), 3.77 (q, J=6.9 Hz, 1H), 3.42 (s, 3H), 3.26 (s, 3H), 3.24 (s, 3H), 3.20 (s, 3H), 3.11 (s, 3H), 2.88 (m, 2H), 2.68 (s, 3H), 2.67 (s, 3H), 2.42 (m, 1H), 2.22-2.10 (m, 5H), 2.01 (s, 3H), 1.92-0.62 (m, 60H); ESI MS m/z 1265 [C₆₃H₁₁₀ClN₁₁O₁₃+H]⁺.

### Example 4

### Preparation of Cyclosporin Vinyl Chloride

Acetyl protected cyclosporin vinyl chloride from Example 3 (20 mg, 0.016 mmol) was dissolved in 4 mL of methanol and, then, K₂CO₃ (100 mg, 0.725 mmol) was added. The mixture was stirred at room temperature overnight, then diluted with 100 mL of EtOAc, washed with brine (3×10 mL), and dried over Na₂SO₄. Solvents were removed in vacuo, and the residue was purified via semi-preparative HPLC to give cyclosporin vinyl chloride (13 mg, 67%) as a white solid:

¹H NMR (CDCl₃, 500 MHz) δ 7.98 (d, J=9.7 Hz, 1H), 7.64 (d, J=7.4 Hz, 1H), 7.42 (d, J=8.4 Hz, 1H), 7.18 (d, J=7.9 Hz, 1H), 5.95 (m, 1H), 5.85 (d, J=13.3 Hz, 1H), 5.46 (d, J=4.5 Hz, 1H), 5.32 (dd, J=11.3, 3.8 Hz, 1H), 5.16-4.94 (m, 5H), 4.85 (t, J=6.9 Hz, 1H), 4.72 (d, J=14.0 Hz, 1H), 4.66 (t, J=8.8 Hz, 1H), 4.54 (t, J=7.2 Hz, 1H), 3.86 (t, J=6.5 Hz, 1H), 3.51 (s, 3H), 3.39 (s, 3H), 3.26 (s, 3H), 3.18 (m, 2H), 3.11 (s, 6H), 2.69 (s, 3H), 2.68 (s, 3H), 2.42 (m, 2H), 2.22-0.62 (m, 65H); ESI MS m/z 1223 [C₆₁H₁₀₈ClN₁₁O₁₂+H]⁺; HPLC 94.6% (AUC), t_{R}=15.3 min.

### Example 5

### Preparation of Acetyl Cyclosporin Vinyl Iodide

To an ice-cooled suspension of chromium (II) chloride (1.0 g, 8.2 mmol) in THF (25 mL) was added a solution of acetyl cyclosporin aldehyde from Example 2 (0.50 g, 0.41 mmol) and iodoform (1.29 g, 3.28 mmol) in THF (25 mL). After 7 h at 0° C., the reaction mixture was poured into ice-water (50 mL). The water layer was extracted with ethyl acetate (3×60 mL). The combined organics were dried over anhydrous sodium sulfate and concentrated. The material was purified by semi-preparative HPLC to afford acetyl cyclosporin vinyl iodide (290 mg, 52%) as a white solid: ¹H NMR (300 MHz, CDCl₃) δ 8.47 (d, J=9.8 Hz, 1H), 8.01 (d, J=6.4 Hz, 1H), 7.56 (d, J=8.8 Hz, 1H), 7.54 (d, J=7.5 Hz, 1H), 6.50-6.40 (m, 1H), 5.84 (d, J=14.3 Hz, 1H), 5.69-5.10 (m, 6H), 4.97 (d, J=11.1 Hz, 2H), 4.87-4.73 (m, 2H), 4.64 (d, J=13.8 Hz, 1H), 4.43 (t, J=7.0 Hz, 1H), 3.43 (s, 3H), 3.28 (s, 3H), 3.26 (s, 3H), 3.20 (s, 3H), 3.12 (s, 3H), 2.68 (s, 3H), 2.66 (s, 3H), 2.45-2.35 (m, 1H), 2.28-1.80 (m, 8H), 2.06 (s, 3H), 1.77-1.60 (m, 3H), 1.50-0.75 (m, 56H); ESI MS m/z 1357 [C₆₃H₁₁₀IN₁₁O₁₃+H]⁺.

### Example 6

### Preparation of Cyclosporin Vinyl Iodide

To a stirred solution of acetyl cyclosporin vinyl iodide from Example 5 (42 mg, 0.030 mmol) in methanol (4 mL) was added potassium carbonate (104 mg, 0.750 mmol) at room temperature. After 12 h at room temperature, methanol was evaporated. Water (20 mL) was added and the mixture was extracted with ethyl acetate (3×70 mL). The organic layer was separated, dried over anhydrous sodium sulfate, and concentrated under vacuum to afford the crude product. The material was purified by semi-preparative HPLC to afford cyclosporin vinyl iodide (30 mg, 78%) as a white solid: ¹H NMR (300 MHz, CDCl₃) δ 7.90 (d, J=9.3 Hz, 1H), 7.66 (d, J=6.5 Hz, 1H), 7.49 (d, J=8.5 Hz, 1H), 7.26 (overlapped with CHCl₃, 1H), 6.55-6.43 (m, 1H), 5.93 (d, J=14.0 Hz, 1H), 5.69 (d, J=8.1 Hz, 1H), 5.47 (d, J=5.9 Hz, 1H), 5.32 (d, J=8.0 Hz, 1H), 5.12-4.92 (m, 4H), 4.82 (t, J=6.2 Hz, 1H), 4.74 (d, J=14.8 Hz, 1H), 4.67 (t, J=9.1 Hz, 1H), 4.53 (t, J=7.2 Hz, 1H), 3.82 (t, J=6.2 Hz, 1H), 3.50 (s, 3H), 3.37 (s, 3H), 3.25 (s, 3H), 3.11 (s, 6H), 2.72 (s, 3H), 2.69 (s, 3H), 2.48-1.90 (m, 8H), 1.80-1.53 (m, 6H), 1.50-0.72 (m, 55H); ESI MS m/z 1315 [C₆₁H₁₀₈IN₁₁O₁₂+H]⁺; HPLC 94.3% (AUC), t_{R}=15.82 min.

### Example 7

### Preparation of the Acetates of cis- and Trans-Deuterated Cyclosporin Vinyl Iodide

A mixture of acetyl cyclosporin aldehyde from Example 2 (500 mg, 0.40 mmol) and iodoform-d (1.35 g, 4.0 mmol) in anhydrous THF (10 mL) was cooled to −78° C. After cooling, chromium chloride (1.0 g, 8.0 mmol) was quickly added to the reaction. The mixture was allowed to warm to 0° C. and stirred under N₂ atmosphere for 5 h. The mixture was poured into ice-water (300 mL) and extracted with ethyl acetate (3×200 mL). Combined organic layers were washed with brine, dried over sodium sulfate, and concentrated in vacuo. The crude product was purified by semi-preparative HPLC to afford the acetate of trans-deuterated cyclosporin vinyl iodide (220 mg, 40%) as a light brown solid: ¹H NMR (300 MHz, CDCl₃) δ 8.47 (d, J=9.6 Hz, 1H), 8.01 (d, J=6.8 Hz, 1H), 7.57 (t, J=8.6 Hz, 2H), 6.44 (dd, J=8.6, 6.1 Hz, 2H), 6.01-5.56 (m, 4H), 5.52 (d, J=10.3 Hz, 2H), 5.28 (d, J=3.4 Hz, 1H), 5.24 (d, J=3.4 Hz, 1H), 4.97 (d, J=10.9 Hz, 3H), 4.85-4.76 (m, 5H), 4.64 (d, J=13.9 Hz, 2H), 4.43 (t, J=7.0 Hz, 2H), 3.43 (s, 3H), 3.25 (s, 3H), 3.24 (s, 3H), 3.20 (s, 3H), 3.11 (s, 3H), 2.67 (s, 3H), 2.66 (s, 3H), 2.01 (s, 2H), 1.32 (d, J=7.1 Hz, 4H), 1.28 (d, J=6.9 Hz, 4H), 1.06-0.74 (m, 52H); ESI MS m/z 1357 [C₆₃H₁₀₉DIN₁₁O₁₃+H]⁺; and the acetate of cis-deuterated cyclosporin vinyl iodide (40 mg, 7%) as a light brown solid: ¹H NMR (300 MHz, CDCl₃) δ 8.57 (d, J=9.6 Hz, 1H), 8.02 (d, J=6.8 Hz, 1H), 7.66 (d, J=9.0 Hz, 1H), 7.53 (d, J=7.7 Hz, 1H), 6.02-5.92 (m, 2H), 5.69 (dd, J=11.0, 3.9 Hz, 1H), 5.54 (d, J=3.9 Hz, 3H), 5.33-5.13 (m, 5H), 4.98 (d, J=11.1 Hz, 3H), 4.82 (t, J=7.3 Hz, 2H), 4.74 (t, J=9.5 Hz, 2H), 4.64 (d, J=13.8 Hz, 2H), 4.32 (t, J=7.0 Hz, 2H), 3.44 (s, 3H), 3.28 (s, 3H), 3.25 (s, 3H), 3.20 (s, 3H), 3.11 (s, 3H), 2.67 (s, 3H), 2.66 (s, 3H), 2.05 (s, 2H), 1.29 (d, J=5.5 Hz, 4H), 1.24 (d, J=11.9 Hz, 4H), 1.05 (d, J=6.4 Hz, 2H), 1.02-0.64 (m, 50H); ESI MS m/z 1357 [C₆₃H₁₀₉DIN₁₁O₁₃+H]⁺.

### Example 8

### Preparation of cis-Deuterated Cyclosporin Vinyl Iodide

A solution of the acetate of cis-deuterated cyclosporin vinyl iodide from Example 7 (40 mg, 0.029 mmol) in methanol (2 mL) was stirred at room temperature. Reaction mixture was treated with potassium carbonate (50 mg, 0.36 mmol) and was allowed to keep stirring under N₂ atmosphere overnight. Mixture was diluted with ethyl acetate, washed with saturated sodium bicarbonate solution and brine, dried over sodium sulfate, and concentrated in vacuo. The crude product was purified by semi-preparative HPLC to afford cis-deuterated cyclosporin vinyl iodide (20 mg, 53%) as a white solid: ¹H NMR (300 MHz, CDCl₃) δ 8.03 (d, J=9.7 Hz, 1H), 7.65 (d, J=7.2 Hz, 1H), 7.47 (d, J=8.3 Hz, 1H), 7.26 (hidden by solvent peak, 1H), 6.17 (dd, J=7.8, 5.6 Hz, 1H), 5.69 (dd, J=10.8, 3.8 Hz, 1H), 5.43 (d, J=7.3 Hz, 2H), 5.31 (dd, J=11.4, 3.8 Hz, 1H), 5.11-4.99 (m, 7H), 4.84 (t, J=7.2 Hz, 2H), 4.83-4.62 (m, 6H), 4.49 (t, J=7.2 Hz, 2H), 3.96 (t, J=6.6 Hz, 2H), 3.51 (s, 3H), 3.40 (s, 3H), 3.24 (s, 3H), 3.12 (s, 3H), 3.11 (s, 3H), 2.69 (s, 3H), 2.68 (s, 3H), 1.35 (d, J=7.2 Hz, 4H), 1.25 (t, J=2.6 Hz, 4H), 1.07-0.81 (m, 50H); ESI MS m/z 1316 [C₆₁H₁₀₇DIN₁₁O₁₂+H]⁺; HPLC>99% (AUC), t_{R}=20.40 min.

### Example 9

### Preparation of Trans-Deuterated Cyclosporin Vinyl Iodide

A solution of the acetate of trans-deuterated cyclosporin vinyl iodide from Example 7 (50 mg, 0.037 mmol) in methanol (2 mL) was stirred at room temperature. Reaction mixture was treated with potassium carbonate (60 mg, 0.43 mmol) and was allowed to keep stirring under N₂ atmosphere overnight. Mixture was diluted with ethyl acetate, washed with saturated sodium bicarbonate solution and brine, dried over sodium sulfate, and concentrated in vacuo. The crude product was purified by semi-preparative HPLC to afford trans-deuterated cyclosporin vinyl iodide (29 mg, 60%) as a white solid: ¹H NMR (300 MHz, CDCl₃) δ 7.95 (d, J=9.8 Hz, 1H), 7.63 (d, J=7.4 Hz, 1H), 7.43 (d, J=8.3 Hz, 1H), 7.17 (d, J=6.2 Hz, 1H), 6.50 (t, J=8.0 Hz, 1H), 5.70 (dd, J=10.9, 3.8 Hz, 1H), 5.49 (d, J=6.2 Hz, 2H), 5.31 (dd, J=10.8, 3.8 Hz, 1H), 5.10-4.62 (m, 15H), 4.50 (t, J=7.2 Hz, 2H), 3.82 (t, J=6.3 Hz, 2H), 3.51 (s, 3H), 3.39 (s, 3H), 3.26 (s, 3H), 3.11 (s, 3H), 3.10 (s, 3H), 2.69 (s, 3H), 2.68 (s, 3H), 1.34 (d, J=7.1 Hz, 4H), 1.25 (d, J=4.7 Hz, 4H), 1.08-0.81 (m, 48H), 0.74 (d, J=8.0 Hz, 2H); ESI MS m/z 1316 [C₆₁H₁₀₇DIN₁₁O₂+H]⁺; HPLC>99% (AUC), t_{R}=20.17 min.

### Example 10

### Preparation of the Acetates of cis- and trans-Deuterated Cyclosporin Vinyl Chloride

A mixture of acetyl cyclosporin aldehyde from Example 2 (500 mg, 0.40 mmol) and chloroform-d (0.32 mL, 4.0 mmol) in anhydrous THF (5 mL) was cooled to −78° C. After cooling, chromium chloride (1.0 g, 8.0 mmol) was quickly added to the reaction. Mixture was allowed to warm to 0° C. and stirred under N₂ atmosphere for 4 h. Mixture was poured into ice-water (300 mL) and extracted with ethyl acetate (3×200 mL). Combined organic layers were washed with brine, dried over sodium sulfate, and concentrated in vacuo. The crude product was purified by semi-preparative HPLC to afford the acetate of trans-deuterated cyclosporin vinyl chloride (136 mg, 27%) as a light brown solid: ¹H NMR (300 MHz, CDCl₃) δ 8.48 (d, J=9.6 Hz, 1H), 8.00 (d, J=6.8 Hz, 1H), 7.65 (d, J=9.1 Hz, 1H), 7.59 (d, J=7.8 Hz, 1H), 5.84 (dd, J=9.3, 5.6 Hz, 2H), 5.68 (dd, J=1.1, 3.8 Hz, 2H), 5.57 (d, J=4.8 Hz, 1H), 5.53 (d, J=8.5 Hz, 1H), 5.48-4.97 (m, 1H), 4.87-4.76 (m, 3H), 4.64 (d, J=12.2 Hz, 2H), 4.43 (t, J=7.0 Hz, 2H), 3.43 (s, 3H), 3.26 (s, 3H), 3.24 (s, 3H), 3.20 (s, 3H), 3.11 (s, 3H), 2.67 (s, 3H), 2.66 (s, 3H), 2.01 (s, 2H), 1.42-1.27 (m, 8H), 1.06-0.74 (m, 50H); ESI MS m/z 1267 [C₆₃H₁₀₉DClN₁₁O₁₃+H]⁺; and the acetate of cis-deuterated cyclosporin vinyl chloride (32 mg, 6%) as a light brown solid: ¹H NMR (300 MHz, CDCl₃) δ 8.54 (d, J=9.6 Hz, 1H), 8.03 (d, J=6.8 Hz, 1H), 7.74 (d, J=9.0 Hz, 1H), 7.58 (d, J=7.7 Hz, 1H), 5.71-5.58 (m, 3H), 5.53 (d, J=7.0 Hz, 2H), 5.40-5.10 (m, 6H), 4.98 (d, J=1.0 Hz, 3H), 4.85 (t, J=7.3 Hz, 2H), 4.75 (t, J=9.5 Hz, 2H), 4.64 (d, J=13.8 Hz, 2H), 4.44 (t, J=7.0 Hz, 2H), 3.44 (s, 3H), 3.27 (s, 3H), 3.22 (s, 3H), 3.19 (s, 3H), 3.11 (s, 3H), 2.68 (s, 3H), 2.67 (s, 3H), 2.04 (s, 2H), 1.34-1.27 (m, 8H), 1.05 (d, J=6.4 Hz, 2H), 1.02-0.79 (m, 50H); ESI MS m/z 1267 [C₆₃H₁₀₉DClN₁₁O₁₃+H]⁺.

### Example 11

### Preparation of Trans-Deuterated Cyclosporin Vinyl Chloride

A solution of the acetate of trans-deuterated cyclosporin vinyl chloride from Example 10 (30 mg, 0.024 mmol) in methanol (2 mL) was stirred at room temperature. Reaction mixture was treated with potassium carbonate (35 mg, 0.25 mmol) and was allowed to keep stirring under N₂ atmosphere overnight. Mixture was diluted with ethyl acetate, washed with saturated sodium bicarbonate solution and brine, dried over sodium sulfate, and concentrated in vacuo. The crude product was purified by semi-preparative HPLC to afford trans-deuterated cyclosporin vinyl chloride (17 mg, 60%) as a white solid: ¹H NMR (300 MHz, CDCl₃) δ 7.96 (d, J=9.6 Hz, 1H), 7.65 (d, J=7.3 Hz, 1H), 7.45 (d, J=8.2 Hz, 1H), 7.21 (d, J=7.8 Hz, 1H), 5.92 (t, J=8.4 Hz, 2H), 5.69 (dd, J=11.0, 4.1 Hz, 1H), 5.48 (d, J=6.4 Hz, 2H), 5.33 (dd, J=11.4, 3.9 Hz, 1H), 5.17-4.62 (m, 16H), 4.51 (t, J=7.2 Hz, 2H), 4.85 (t, J=6.4 Hz, 2H), 3.51 (s, 3H), 3.39 (s, 3H), 3.26 (s, 3H), 3.11 (s, 6H), 2.70 (s, 3H), 2.68 (s, 3H), 1.34 (d, J=7.2 Hz, 2H), 1.26 (d, J=6.6 Hz, 2H), 1.08-0.75 (m, 50H), 0.66 (d, J=5.2 Hz, 2H); ESI MS m/z 1224 [C₆₁H₁₀₇DClN₁₁O₁₂+H]⁺; HPLC>99% (AUC), t_{R}=19.57 min.

### Example 12

### Preparation of cis-Deuterated Cyclosporin Vinyl Chloride

A solution of the acetate of cis-deuterated cyclosporin vinyl chloride from example 10 (32 mg, 0.025 mmol) in methanol (2 mL) was stirred at room temperature. Reaction mixture was treated with potassium carbonate (38 mg, 0.27 mmol) and was allowed to keep stirring under N₂ atmosphere overnight. Mixture was diluted with ethyl acetate, washed with saturated sodium bicarbonate solution and brine, dried over sodium sulfate, and concentrated in vacuo. The crude product was purified by semi-preparative HPLC to afford cis-deuterated cyclosporin vinyl chloride (17 mg, 55%) as a white solid: ¹H NMR (300 MHz, CDCl₃) δ 8.05 (d, J=9.7 Hz, 1H), 7.68 (d, J=7.1 Hz, 1H), 7.50 (d, J=8.3 Hz, 1H), 7.28 (d, J=9.0 Hz, 1H), 5.83-5.77 (m, 2H), 5.69 (dd, J=10.9, 4.0 Hz, 1H), 5.42 (d, J=7.3 Hz, 2H), 5.29 (dd, J=11.3, 3.8 Hz, 1H), 5.11-4.98 (m, 7H), 4.82 (t, J=7.3 Hz, 2H), 4.78-4.62 (m, 7H), 4.49 (t, J=7.1 Hz, 2H), 3.94 (t, J=6.7 Hz, 2H), 3.51 (s, 3H), 3.40 (s, 3H), 3.24 (s, 3H), 3.12 (s, 3H), 3.11 (s, 3H), 2.69 (s, 3H), 2.68 (s, 3H), 1.34 (d, J=7.2 Hz, 2H), 1.26 (d, J=6.6 Hz, 2H), 1.07-0.76 (m, 52H); ESI MS m/z 1224 [C₆₁H₁₀₇DClN₁₁O₁₂+H]⁺; HPLC>99% (AUC), t_{R}=19.82 min.

### Example 13

### Preparation of the Acetates of cis- and trans-Cyclosporin Vinyl Chloride

NaHMDS (1.0 M in THF, 0.4 mL, 0.4 mmol) was added to a mixture of (chloromethyl)triphenylphosphonium chloride (140 mg, 0.4 mmol) and 4 mL of THF at −78° C. under nitrogen, the mixture was stirred at −78° C. for 1 h, followed by the addition of a solution of acetyl cyclosporin aldehyde from Example 2 (100 mg, 0.08 mmol) in 3 mL of THF. The resulted mixture was stirred at −78° C. for 2 h, quenched with 4 mL of saturated aqueous NH₄Cl, extracted with ether (3×30 mL). Combined organic layers were washed with brine, dried over Na₂SO₄. After that, the solvent was removed in vacuo, and the residue was purified via semi-preparative HPLC to give the acetate of the cis-isomer of cyclosporin vinyl chloride (13 mg, 12%) as a white solid: ¹H NMR (CDCl₃, 300 MHz) δ 8.56 (d, J=9.6 Hz, 1H), 8.03 (d, J=6.9 Hz, 1H), 7.67 (d, J=9.3 Hz, 1H), 7.54 (d, J=7.8 Hz, 1H), 5.92 (d, J=6.9 Hz, 1H), 5.67 (m, 2H), 5.53 (d, J=6.0 Hz, 2H), 5.28-5.11 (m, 10H), 4.97 (d, J=11.1 Hz, 3H), 4.85 (t, J=7.2 Hz, 1H), 4.74 (t, J=6.6 Hz, 1H), 4.63 (d, J=13.8 Hz, 1H), 4.43 (t, J=6.9 Hz, 1H), 3.44 (s, 3H), 3.27 (s, 3H), 3.24 (m, 2H), 3.23 (s, 3H), 3.20 (s, 3H), 3.09 (s, 3H), 2.67 (s, 3H), 2.65 (s, 3H), 2.42 (m, 1H), 2.23-2.10 (m, 6H), 2.00 (s, 3H), 1.98-0.62 (m, 51H); ESI MS m/z 1265 [C₆₃H₁₁₀ClN₁₁O₁₃+H]⁺; and the acetate of the trans-isomer of cyclosporin vinyl chloride (10 mg, 9.7%) as a white solid: ¹H NMR (CDCl₃, 500 MHz) δ 8.46 (d, J=9.3 Hz, 1H), 8.00 (d, J=6.9 Hz, 1H), 7.64 (d, J=9.0 Hz, 1H), 7.58 (d, J=7.8 Hz, 1H), 5.83 (m, 1H), 5.68 (d, J=7.2 Hz, 1H), 5.56 (d, J=11.1 Hz, 2H), 5.44 (d, J=13.5, 3.8 Hz, 1H), 5.22 (m, 1H), 5.06-4.94 (m, 3H), 4.85 (t, J=7.2 Hz, 1H), 4.77 (d, J=10.6 Hz, 1H), 4.64 (d, J=13.5 Hz, 1H), 4.43 (t, J=6.6 Hz, 1H), 3.77 (q, J=6.9 Hz, 1H), 3.42 (s, 3H), 3.26 (s, 3H), 3.24 (s, 3H), 3.20 (s, 3H), 3.11 (s, 3H), 2.88 (m, 2H), 2.68 (s, 3H), 2.67 (s, 3H), 2.42 (m, 1H), 2.22-2.10 (m, 5H), 2.01 (s, 3H), 1.92-0.62 (m, 60H); ESI MS m/z 1265 [C₆₃H₁₁₀ClN₁₁O₁₃+H]⁺.

### Example 14

### Preparation of the cis-Isomer of Cyclosporin Vinyl Chloride

The acetate of the cis-isomer of cyclosporin vinyl chloride from Example 13 (13 mg, 0.01 mmol) was dissolved in 3 mL of methanol, and then K₂CO₃ (50 mg, 0.36 mmol) was added. The mixture was stirred at room temperature overnight, then diluted with 100 mL of EtOAc, washed with brine (3×10 mL), dried over Na₂SO₄. Solvents were removed in vacuo, and the residue was purified via semi-preparative HPLC to give the cis-isomer of cyclosporin chloride (9 mg, 71%) as a white solid: ¹H NMR (CDCl₃, 500 MHz) δ 8.04 (d, J=9.7 Hz, 1H), 7.68 (d, J=7.3 Hz, 1H), 7.49 (d, J=8.5 Hz, 1H), 7.27 (d, J=9.8 Hz, 1H), 6.98 (s, 1H), 5.99 (d, J=7.1 Hz, 1H), 5.80 (q, J=7.6 Hz, 1H), 5.68 (dd, J=11.0, 4.2 Hz, 1H), 5.42 (d, J=7.4 Hz, 1H), 5.28(dd, J=11.4, 3.8 Hz, 1H), 5.10 (m, 2H), 5.06-4.94 (m, 3H), 4.82 (t, J=7.2 Hz, 1H), 4.70 (d, J=14.0 Hz, 1H), 4.66 (t, J=9.2 Hz, 1H), 4.50 (t, J=7.2 Hz, 1H), 3.95 (t, J=6.5 Hz, 1H), 3.51 (s, 3H), 3.40 (s, 3H), 3.25 (s, 3H), 3.20 (m, 2H), 3.12 (s, 3H), 3.11 (s, 3H), 2.70 (s, 3H), 2.69 (s, 3H), 2.42 (m, 2H), 2.22-0.62 (m, 63H); ESI MS m/z 1223 [C₆₁H₁₀₈ClN₁₁O₁₂+H]⁺; HPLC 94.6% (AUC), t_{R}=15.8 min.

### Example 15

### Preparation of the Acetate of cis-Cyclosporin Vinyl Iodide

To a vigorously stirred suspension of (iodomethyl)triphenylphosphonium iodide (1.3 g, 2.4 mmol) in dry THF (18 mL) under nitrogen, was added sodium bis(trimethylsilyl)amide (2.4 mL, 1 M in THF, 2.4 mmol). After 10 min at room temperature, the mixture was cooled to 0° C. and acetyl cyclosporin aldehyde from Example 2 (300 mg, 0.240 mmol) in anhydrous THF (10 mL) was added dropwise. After 10 min at 0° C., the reaction was quenched with a saturated solution of ammonium chloride (10 mL), and then allowed to warm to room temperature. The resulting solid was filtered off through a plug of diatomaceous earth and washed with ethyl acetate (200 mL). The organic layer was washed with an aqueous solution of sodium hydrogensulfite (20%, 200 mL), then dried over anhydrous sodium sulfate and concentrated under vacuum to afford the crude product (540 mg). The material was purified by semi-preparative HPLC to afford the acetate of the cis-isomer of cyclosporin vinyl iodide (150 mg, 46%) as a pale-brown oil: ¹H NMR (300 MHz, CDCl₃) δ 8.53 (d, J=9.8 Hz, 1H), 8.15 (d, J=6.5 Hz, 1H), 7.82 (d, J=9.0 Hz, 1H), 7.63 (d, J=8.2 Hz, 1H), 6.10 (d, J=7.4 Hz, 1H), 6.02-5.94 (m, 1H), 5.69 (dd, J=10.9, 3.8 Hz, 1H), 5.61-5.48 (m, 2H), 5.38-5.13 (m, 3H), 4.98 (d, J=10.9 Hz, 2H), 4.87 (t, J=7.4 Hz, 1H), 4.78 (t, J=9.6 Hz, 1H), 4.63 (d, J=14.2 Hz, 1H), 4.47 (t, J=7.0 Hz, 1H), 3.98 (s, 3H), 3.43 (s, 3H), 3.27 (s, 3H), 3.21 (s, 3H), 3.19 (s, 3H), 3.14 (s, 3H), 2.69 (s, 3H), 2.42-2.30 (m, 1H), 2.22-1.85 (m, 8H), 2.06 (s, 3H), 1.77-1.60 (m, 3H), 1.54-0.75 (m, 56H); ESI MS m/z 1357 [C₆₃H₁₁₀IN₁₁O₁₃+H]⁺.

### Example 16

### Preparation of cis-Cyclosporin Vinyl Iodide

To a stirred solution of the acetate of the cis-isomer of cyclosporin vinyl iodide from Example 15 (70 mg, 0.052 mmol) in methanol (8 mL) was added potassium carbonate (180 mg, 1.30 mmol) at room temperature. After 12 h at room temperature, methanol was evaporated. Water (20 mL) was added and the mixture was extracted with ethyl acetate (3×70 mL). The organic layer was separated, dried over anhydrous sodium sulfate, and concentrated under vacuum to afford the crude product (47 mg). The material was purified by semi-preparative HPLC to afford cis-cyclosporin vinyl iodide (−19 mg, 28%) as a white solid: ¹H NMR (300 MHz, CDCl₃) δ 8.01 (d, J=9.8 Hz, 1H), 7.65 (d, J=7.2 Hz, 1H), 7.48 (d, J=8.4 Hz, 1H), 7.27 (d, J=7.8 Hz, 1H), 6.17 (s, 2H), 5.69 (dd, J=11.0, 4.0 Hz, 1H), 5.43 (d, J=7.3 Hz, 1H), 5.31 (dd, J=11.3, 3.7 Hz, 1H), 5.12-4.95 (m, 4H), 4.84 (t, J=7.5 Hz, 1H), 4.71 (d, J=13.7 Hz, 1H), 4.67 (t, J=9.5 Hz, 1H), 4.47 (t, J=7.0 Hz, 1H), 3.96 (t, J=6.7 Hz, 1H), 3.52 (s, 3H), 3.40 (s, 3H), 3.25 (s, 3H), 3.12 (s, 6H), 2.69 (s, 6H), 2.48-1.95 (m, 8H), 1.89-1.53 (m, 6H), 1.50-0.72 (m, 55H); ESI MS m/z 1315 [C₆₁H₁₀₈IN₁₁O₁₂+H]⁺; HPLC 89.7% (AUC), t_{R}=24.46 min.

### Example 17

### Preparation of the Acetates of cis- and trans-Cyclosporin Vinyl Bromide

NaHMDS (1.0 M in THF, 0.8 mL, 0.8 mmol) was added to a mixture of (bromomethyl)triphenyl phosphonium bromide (348 mg, 0.8 mmol) and 8 mL of THF at −78° C. under nitrogen, the mixture was stirred at −78° C. for 1 h, followed by the addition of a solution of acetyl cyclosporin aldehyde from Example 2 (200 mg, 0.16 mmol) in 4 mL of THF. The resulted mixture was stirred −78° C. for 2 h, quenched with 8 mL of saturated aqueous NH₄Cl, extracted with ether (3×30 mL). Combined organic layers were washed with brine, dried over Na₂SO₄. After that, the solvent was removed in vacuo, and the residue was purified via semi-preparative HPLC to give the acetate of the trans-isomer of cyclosporin vinyl bromide (4 mg, 1.9%) as a white solid: ¹H NMR (CDCl₃, 500 MHz) δ 8.50 (d, J=9.8 Hz, 1H), 7.98 (d, J=6.9 Hz, 1H), 7.48 (d, J=8.1 Hz, 1H), 7.41 (d, J=9.2 Hz, 1H), 6.18 (m, 1H), 5.86 (d, J=13.6 Hz, 1H), 5.68 (dd, J=11.2, 4.6 Hz, 1H), 5.52 (q, J=12.1 Hz, 2H), 5.40 (dd, J=11.6, 4.1 Hz, 1H), 5.28 (dd, J=14.3, 3.6 Hz, 1H), 5.14 (dd, J=13.8, 6.0 Hz, 1H), 4.96 (dd, J=16.5, 5.5 Hz, 2H), 4.84 (t, J=7.4 Hz, 1H), 4.77 (t, J=9.6 Hz, 1H), 4.64 (d, J=13.8 Hz, 1H), 4.41 (t, J=7.0 Hz, 1H), 3.43 (s, 3H), 3.27 (s, 3H), 3.26 (s, 3H), 3.23 (s, 3H), 3.15 (m, 2H), 3.10 (s, 3H), 2.67 (s, 3H), 2.65 (s, 3H), 2.42 (m, 1H), 2.22-0.62 (m, 68H); ESI MS m/z 1309 [C₆₃H₁₀N₁₁O₁₃+H]⁺; and the acetate of the cis-isomer of cyclosporin vinyl bromide (13 mg, 6.1%) as a white solid:

¹H NMR (CDCl₃, 300 MHz) δ 8.57 (d, J=9.6 Hz, 1H), 8.04 (d, J=6.6 Hz, 1H), 7.76 (d, J=9.1 Hz, 1H), 7.59 (d, J=7.8 Hz, 1H), 6.04 (m, 2H), 5.70 (dd, J=10.8, 3.6 Hz, 1H), 5.53 (t, J=11.7 Hz, 1H), 5.32-5.14 (m, 4H), 5.01 (d, J=8.1 Hz, 2H), 4.86 (t, J=7.2 Hz, 1H), 4.76 (t, J=9.6 Hz, 1H), 4.63 (d, J=14.1 Hz, 1H), 4.44 (t, J=6.6 Hz, 1H), 3.45 (s, 3H), 3.30 (s, 3H), 3.27 (s, 3H), 3.26 (s, 3H), 3.24 (m, 2H), 3.12 (s, 3H), 2.68 (s, 3H), 2.67 (s, 3H), 2.42 (m, 1H), 2.22-0.62 (m, 68H); ESI MS m/z 1309 [C₆₃H₁₁₀N₁₁O₁₃+H]⁺.

### Example 18

### Preparation of cis-Cyclosporin Vinyl Bromide

The acetate of the cis-isomer of cyclosporin vinyl bromide from Example 17 (13 mg, 0.01 mmol) was dissolved in 3 mL of methanol, and then K₂CO₃ (50 mg, 0.36 mmol) was added. The mixture was stirred at room temperature overnight, then diluted with 100 mL of EtOAc, washed with brine (3×10 mL), dried over Na₂SO₄. Solvents were removed in vacuo, the residue was purified via semi-preparative HPLC to give the cis-cyclosporin vinyl bromide (5.1 mg, 40%) as a white solid: ¹H NMR (CDCl₃, 500 MHz) δ 8.05 (d, J=9.8 Hz, 1H), 7.67 (d, J=7.2 Hz, 1H), 7.46 (d, J=8.3 Hz, 1H), 7.23 (d, J=9.2 Hz, 2H), 6.98 (s, 1H), 6.10 (m, 2H), 5.68 (dd, J=10.9, 4.3 Hz, 1H), 5.42 (d, J=7.5 Hz, 1H), 5.28 (dd, J=11.5, 4.0 Hz, 1H), 5.10-5.00 (m, 4H), 4.82 (t, J=6.8 Hz, 1H), 4.69 (d, J=13.9 Hz, 1H), 4.65 (t, J=9.4 Hz, 1H), 4.49 (t, J=7.2 Hz, 1H), 3.96 (t, J=6.1 Hz, 1H), 3.51 (s, 3H), 3.41 (s, 3H), 3.25 (s, 3H), 3.18 (m, 2H), 3.12 (s, 3H), 3.10 (s, 3H), 2.70 (s, 3H), 2.68 (s, 3H), 2.42 (m, 1H), 2.22-0.62 (m, 64H); ESI MS m/z 1267 [C₆₁H₁₀₈N₁₁O₁₂+H]⁺; HPLC 98.3% (AUC), t_{R}=16.1 min.

### Example 19

### Preparation of trans-Cyclosporin Vinyl Bromide

The acetate of the trans-isomer of cyclosporin vinyl bromide from Example 17 (7 mg, 0.005 mmol) was dissolved in 3 mL of methanol, and then K₂CO₃ (50 mg, 0.36 mmol) was added. The mixture was stirred at room temperature overnight, then diluted with 100 mL of EtOAc, washed with brine (3×10 mL), dried over Na₂SO₄. Solvents were removed in vacuo, and the residue was purified by semi-preparative HPLC to give trans-cyclosporin vinyl bromide (4.0 mg, 55%) as a white solid: ¹H NMR (CDCl₃, 500 MHz) δ 7.96 (d, J=8.5 Hz, 1H), 7.63 (d, J=7.0 Hz, 1H), 7.41 (d, J=8.6 Hz, 1H), 7.17 (d, J=8.2 Hz, 1H), 6.20 (ddd, J=22.4, 13.8, 8.9 Hz, 1H), 5.94 (d, J=13.3 Hz, 1H), 5.70 (dd, J=11.2, 4.2 Hz, 1H), 5.49 (d, J=6.5 Hz, 1H), 5.32 (dd, J=7.9, 4.0 Hz, 1H), 5.13-5.02 (m, 4H), 4.96 (dd, J=10.2, 5.6 Hz, 1H), 4.83 (t, J=7.2 Hz, 1H), 4.72 (d, J=13.6 Hz, 1H), 4.65 (dd, J=17.7, 9.0 Hz, 1H), 4.61 (t, J=7.2 Hz, 1H), 3.85 (t, J=6.1 Hz, 1H), 3.51 (s, 3H), 3.39 (s, 3H), 3.26 (s, 3H), 3.18 (d, J=14.2 Hz, 2H), 3.11 (s, 6H), 2.70 (s, 3H), 2.68 (s, 3H), 2.45-2.40 (m, 2H), 2.12-2.02 (m, 7H), 1.85-0.76 (m, 57H); ESI MS m/z 1267 [C₆₁H₁₀₈BrN₁₁O₁₂+H]⁺; HPLC 98.9% (AUC), t_{R}=16.4 min.

### Example 20

### Preparation of the Acetate of Cyclosporin Vinyl Dichloride

To a mixture of acetyl cyclosporin aldehyde from Example 2 (150 mg, 0.120 mmol) and triphenylphosphine (630 mg, 2.40 mmol) in acetonitrile (2 mL) was added carbon tetrachloride (0.12 mL, 1.2 mmol) in one portion at 0° C. The mixture was allowed to warm to room temperature. After 2 h at room temperature, water (5 mL) was added into the solution and then the mixture was extracted with ethyl acetate (100 mL). The organic layer washed with brine (20 mL), dried over anhydrous sodium sulfate, and concentrated. The crude product was purified by semi-preparative HPLC to afford the acetate of cyclosporin vinyl dichloride (50 mg, 32%) as a white solid: ¹H NMR (300 MHz, CDCl₃) δ 8.49 (d, J=9.7 Hz, 1H), 8.01 (d, J=6.8 Hz, 1H), 7.60 (app t, J=7.2 Hz, 2H), 5.99 (dd, J=8.9, 5.6 Hz, 1H), 5.68 (dd, J=11.0, 3.9 Hz, 1H), 5.60-5.45 (m, 2H), 5.41 (dd, J=12.0, 3.8 Hz, 1H), 5.30-4.75 (m, 6H), 4.63 (d, J=14.0 Hz, 1H), 4.43 (t, J=7.0 Hz, 1H), 3.43 (s, 3H), 3.28 (s, 3H), 3.26 (s, 3H), 3.20 (s, 3H), 3.11 (s, 3H), 2.68 (s, 3H), 2.67 (s, 3H), 2.45-2.35 (m, 1H), 2.28-1.85 (m, 8H), 2.03 (s, 3H), 1.75-1.60 (m, 3H), 1.45-0.75 (m, 56H); ESI MS m/z 1299 [C₆₃H₁₀₉Cl₂N₁₁O₁₃+H]⁺.

### Example 21

### Preparation of Cyclosporin Vinyl Dichloride

To a stirred solution of the acetate of cyclosporin vinyl dichloride from Example 20 (45 mg, 0.040 mmol) in methanol (4 mL) was added potassium carbonate (121 mg, 0.870 mmol) at room temperature. After 12 h at room temperature, methanol was evaporated. Water (20 mL) was added and the mixture was extracted with ethyl acetate (3×70 mL). The organic layer was separated, dried over anhydrous sodium sulfate, and concentrated under vacuum to afford the crude product. The material was purified by semi-preparative HPLC to afford cyclosporin vinyl dichloride (25 mg, 57%) as an off-white solid: ¹H NMR (300 MHz, CDCl₃) δ 7.98 (d, J=9.9 Hz, 1H), 7.69 (d, J=7.2 Hz, 1H), 7.42 (d, J=8.5 Hz, 1H), 7.28 (overlapped with CHCl₃, 1H), 6.02 (t, J=8.1 Hz, 1H), 5.69 (dd, J=7.0, 3.9 Hz, 1H), 5.43 (d, J=8.3 Hz, 1H), 5.34 (dd, J=11.7, 3.8 Hz, 1H), 5.15-4.95 (m, 4H), 4.83 (t, J=7.0 Hz, 1H), 4.7144.63 (m, 2H), 4.49 (t, J=7.2 Hz, 1H), 3.92 (t, J=6.5 Hz, 1H), 3.50 (s, 3H), 3.39 (s, 3H), 3.26 (s, 3H), 3.13 (s, 3H), 3.11 (s, 3H), 2.70 (s, 3H), 2.68 (s, 3H), 2.48-2.34 (m, 2H), 2.20-1.92 (m, 6H), 1.80-1.53 (m, 6H), 1.50-0.75 (m, 55H); ESI MS m/z 1257 [C₆₁H₁₀₇Cl₂N₁₁O₁₂+H]⁺; HPLC 97.3% (AUC), t_{R}=16.42 min.

### Example 22

### Preparation of the Acetates of cis- and trans-Cyclosporin Phenylvinyl Chloride

To a solution of diethyl benzylphosphonate (0.50 mL, 2.4 mmol) in THF (2 mL) at −78° C. was added n-butyllithium (1.1 mL, 2.5 M in hexane, 2.6 mmol) dropwise. After 15 min at −78° C., a solution of carbon tetrachloride (0.23 mL, 2.4 mmol) in THF (1 mL) was added. After 15 min at −78° C., a solution of acetyl cyclosporin aldehyde from Example 2 (150 mg, 0.120 mmol) in THF (1 mL) was added. After 15 min at −78° C., the reaction was allowed to warm to room temperature over 1 h. The reaction was quenched with water (2 mL), and then extracted with ethyl acetate (2×50 mL). The combined organics were dried over anhydrous sodium sulfate and concentrated. The crude product was purified by semi-preparative HPLC to afford the acetate of cyclosporin phenylvinyl chloride (118 mg, 73%) as a mixture of cis and trans-isomers and a pale-brown solid: ¹H NMR (300 MHz, CDCl₃) δ 8.49 (d, J=9.0 Hz, 1H), 8.07 (d, J=6.5 Hz, 0.5H), 8.00 (d, J=6.5 Hz, 0.5H), 7.76 (d, J=8.9 Hz, 1H), 7.66 (d, J=8.1 Hz, 0.5H), 7.61 (d, J=8.1 Hz, 0.5H), 7.46-7.28 (m, 5H), 6.00 (t, J=5.7 Hz, 1H), 5.68-4.82 (m, 10H), 4.70-4.40 (m, 2H), 3.45 (s, 3H), 3.25 (s, 3H), 3.19 (s, 1.5H), 3.18 (s, 1.5H), 3.15 (s, 3H), 3.07 (s, 3H), 2.70 (s, 1.5H), 2.68 (s, 1.5H), 2.66 (s, 1.5H), 2.64 (s, 1.5H), 2.45-2.35 (m, 1H), 2.20-1.85 (m, 8H), 2.03 (s, 3H), 1.75-1.55 (m, 3H), 1.45-0.50 (m, 56H); ESI MS m/z 1341 [C₆₉H₁₁₄ClN₁₁O₁₃+H]⁺.

### Example 23

### Preparation of cis- and trans-Cyclosporin Phenylvinyl Chloride

To a stirred solution of the acetate of cyclosporin phenylvinyl chloride from Example 22 (59 mg, 0.040 mmol) in methanol (5 mL) was added potassium carbonate (149 mg, 1.07 mmol) at room temperature. After 12 h at room temperature, methanol was evaporated. Water (20 mL) was added and the mixture was extracted with ethyl acetate (3×70 mL). The organic layer was separated, dried over anhydrous sodium sulfate, and concentrated under vacuum to afford the crude product. The material was purified by semi-preparative HPLC to afford cyclosporin phenylvinyl chloride (35 mg, 63%) as a mixture of cis- and trans-isomers and a white solid: ¹H NMR (300 MHz, CDCl₃) δ 8.10-7.92 (m, 1H), 7.70 (br s, 1H), 7.54 (d, J=6.9 Hz, 2H), 7.38-7.28 (m, 5H), 6.18 (t, J=6.2 Hz, 0.5H), 6.05 (t, J=6.2 Hz, 0.5H), 5.69 (d, J=8.6 Hz, 1H), 5.55 (br s, 1H), 5.37 (dd, J=13.2, 3.8 Hz, 1H), 5.15-4.45 (m, 8H), 3.81 (br s, 1H), 3.53 (s, 1.5H), 3.49 (s, 1.5H), 3.40 (s, 1.5H), 3.38 (s, 1.5H), 3.26 (s, 3H), 3.11 (s, 6H), 2.72 (s, 3H), 2.69 (s, 3H), 2.48-1.53 (m, 14H), 1.50-0.75 (m, 55H); ESI MS m/z 1299 [C₆₇H₁₁₂ClN₁₁O₁₂+H]⁺; HPLC>99% (AUC), t_{R}=16.65 min.

### Example 24

### Preparation of the Acetate of Cyclosporin α,β-Unsaturated Aldehyde

A mixture of acetyl-protected cyclosporin A from Example 1 (100 mg, 0.08 mmol), acrolein dimethyl acetal (0.018 mL, 0.16 mmol), Grubbs' catalyst 2^{nd} generation (25 mg, 0.029 mmol) and methylene chloride (1 mL) was heated at 60° C. in a sealed tube for 12 h. The catalyst (25 mg) and acrolein dimethyl acetal (0.018 mL) were refilled, and the mixture was stirred at the same temperature for an additional 12 h, cooled to room temperature, and concentrated in vacuo. The residue was purified by semi-preparative HPLC to afford the acetate of cyclosporin α,β-unsaturated aldehyde (65 mg, 64%) as an off-white solid: ¹H NMR (300 MHz, CDCl₃) δ 9.42 (d, J=7.9 Hz, 1H), 8.55 (d, J=9.6 Hz, 1H), 8.02 (d, J=6.8 Hz, 1H), 7.71 (d, J=8.8 Hz, 1H), 7.53 (d, J=7.5 Hz, 1H), 6.73 (ddd, J=15.5, 10.0, 4.5 Hz, 1H), 5.60 (dd, J=15.5, 7.9 Hz, 1H), 5.70-4.40 (m, 12H), 3.46 (s, 3H), 3.27 (s, 3H), 3.22 (s, 3H), 3.21 (s, 3H), 3.13 (s, 3H), 2.68 (s, 3H), 2.66 (s, 3H), 2.50-1.50 (m, 10H), 2.04 (s, 3H), 1.40-0.75 (m, 58H); ESI MS m/z 1259 [C₆₄H₁₁₁N₁₁O₁₄+H]⁺.

### Example 25

### Preparation of the Acetate of Cyclosporin Vinyl Chloride

Chromium (II) chloride (235 mg, 1.92 mmol) was added to a solution of the acetate of cyclosporin α,β-unsaturated aldehyde from Example 24 (80 mg, 0.64 mmol) and chloroform (0.05 mL, 0.064 mmol) in THF (3 mL) at room temperature. The mixture was stirred at 50° C. for 4 h and then cooled to room temperature, quenched with water, extracted with ethyl acetate. The combined organic layers were washed with brine, dried over sodium sulfate, filtered, and concentrated in vacuo. The residue was purified by semi-preparative HPLC to afford the acetate of cyclosporin vinyl chloride (25 mg, 30%) as a white solid: ¹H NMR (300 MHz, CDCl₃) δ 8.55 (t, J=9.4 Hz, 1H), 8.05 (t, J=6.8 Hz, 1H), 7.76 (t, J=9.2 Hz, 1H), 7.55 (d, J=7.3 Hz, 1H), 6.42 (dd, J=13.1, 10.8 Hz, 1H), 6.06 (d, J=13.1 Hz, 1H), 5.82 (dd, J=15.0, 10.6 Hz, 1H), 5.70-4.35 (m, 13H), 3.44 (s, 3H), 3.25 (s, 3H), 3.23 (s, 3H), 3.20 (s, 3H), 3.11 (s, 3H), 2.68 (s, 3H), 2.66 (s, 3H), 2.50-1.50 (m, 10H), 2.02 (s, 3H), 1.40-0.82 (m, 58H); ESI MS m/z 1291 [C₆₅H₁₁₂ClN₁₁O₁₃+H]⁺.

### Example 26

### Preparation of Cyclosporin Vinyl Chloride

A mixture of the acetate of cyclosporin vinyl chloride from Example 25 (25 mg, 0.019 mmol), potassium carbonate (50 mg, 0.36 mmol) and methanol (1 mL) was stirred at room temperature overnight, and then diluted with ethyl acetate, washed with water and brine, dried over sodium sulfate, filtered, and concentrated in vacuo. The residue was purified by semi-preparative HPLC to afford cyclosporin vinyl chloride (15 mg, 61%) as a white solid: ¹H NMR (300 MHz, CDCl₃) δ 7.98 (d, J=9.2 Hz, 1H), 7.63 (d, J=7.4 Hz, 1H), 7.57 (t, J=7.7 Hz, 1H), 7.22 (d, J=7.8 Hz, 1H), 6.43 (dd, J=13.1, 10.8 Hz, 1H), 6.07 (d, J=13.1 Hz, 1H), 5.91 (dd, J=15.0, 10.8 Hz, 1H), 5.72-3.80 (m, 13H), 3.50 (s, 3H), 3.39 (s, 3H), 3.25 (s, 3H), 3.11 (s, 3H), 3.10 (s, 3H), 2.70 (s, 3H), 2.69 (s, 3H), 2.50-1.50 (m, 1H), 1.40-0.82 (m, 58H); ESI MS m/z 1249 [C₆₃H₁₁₀ClN₁₁O₁₂+H]⁺; HPLC>99% (AUC), t_{R}=14.43 min.

### Example 27

### Preparation of the Acetate of Cyclosporin Vinyl Bromide

Chromium (II) chloride (235 mg, 1.92 mmol) was added to a solution of the acetate of cyclosporin α,β-unsaturated aldehyde from Example 24 (80 mg, 0.064 mmol) and bromoform (0.084 mL, 0.96 mmol) in THF (3 mL) at room temperature. The mixture was stirred under nitrogen for 4 h and then quenched with water, extracted with ethyl acetate. The combined organic layers were washed with brine, dried over sodium sulfate, filtered, and concentrated in vacuo. The residue was purified by semi-preparative HPLC to afford the acetate of cyclosporin vinyl bromide (13 mg, 15%) as a white solid: ¹H NMR (300 MHz, CDCl₃) δ 8.55 (d, J=9.4 Hz, 1H), 8.05 (d, J=6.8 Hz, 1H), 7.71 (d, J=9.2 Hz, 1H), 7.51 (d, J=7.7 Hz, 1H), 6.70 (dd, J=13.4, 10.8 Hz, 1H), 6.16 (d, J=13.4 Hz, 1H), 5.81 (dd, J=15.5, 11.1 Hz, 1H), 5.70-4.35 (m, 13H), 3.44 (s, 3H), 3.25 (s, 3H), 3.23 (s, 3H), 3.20 (s, 3H), 3.11 (s, 3H), 2.68 (s, 3H), 2.66 (s, 3H), 2.50-1.50 (m, 10H), 2.02 (s, 3H), 1.40-0.82 (m, 58H); ESI MS m/z 1334 [C₆₅H₁₁₂BrN₁₁O₁₃+H]⁺.

### Example 28

### Preparation of Cyclosporin Vinyl Bromide

A mixture of the acetate of cyclosporin vinyl bromide from Example 27 (13 mg, 0.01 mmol), potassium carbonate (30 mg, 0.22 mmol) and methanol (1 mL) was stirred at room temperature overnight, and then diluted with ethyl acetate, washed with water and brine, dried over sodium sulfate, filtered, and concentrated in vacuo. The residue was purified by semi-preparative HPLC to afford cyclosporin vinyl bromide (4 mg, 31%) as a white solid: ¹H NMR (300 MHz, CDCl₃) δ 7.95 (d, J=9.8 Hz, 1H), 7.64 (d, J=7.4 Hz, 1H), 7.53 (t, J=8.1 Hz, 1H), 7.20 (d, J=7.4 Hz, 1H), 6.70 (dd, J=13.1, 10.7 Hz, 1H), 6.17 (d, J=13.4 Hz, 1H), 5.90 (dd, J=15.0, 10.7 Hz, 1H), 5.72-3.75 (m, 13H), 3.50 (s, 3H), 3.39 (s, 3H), 3.25 (s, 3H), 3.11 (s, 3H), 3.10 (s, 3H), 2.70 (s, 3H), 2.69 (s, 3H), 2.50-1.50 (m, 1H), 1.40-0.82 (m, 58H); ESI MS m/z 1292 [C₆₃H₁₁₀BrN₁₁O₁₂+H]⁺; HPLC>99% (AUC), t_{R}=14.30 min.

### Example 29

### Preparation of the Acetate of Cyclosporin Vinyl Iodide

Chromium(II) chloride (340 mg, 2.76 mmol) was added to a solution of the acetate of cyclosporin α,β-unsaturated aldehyde from Example 24 (174 mg, 0.138 mmol) and iodoform (540 mg, 1.38 mmol) in THF (5 mL) at −40° C. The mixture was allowed to warm to 0° C. and stirred under nitrogen for 1 h. The mixture was poured into ice water, extracted with ethyl acetate. The combined organic layers were washed with brine, dried over sodium sulfate, filtered, and concentrated in vacuo. The residue was purified by semi-preparative HPLC to afford the acetate of cyclosporin vinyl iodide (125 mg, 65%) as a light yellow solid: ¹H NMR (300 MHz, CDCl₃) δ 8.55 (d, J=9.4 Hz, 1H), 8.07 (d, J=6.8 Hz, 1H), 7.84 (d, J=9.2 Hz, 1H), 7.59 (d, J=7.3 Hz, 1H), 6.95 (dd, J=14.2, 10.6 Hz, 1H), 6.16 (d, J=14.4 Hz, 1H), 5.82 (dd, J=14.9, 10.6 Hz, 1H), 5.70-4.40 (m, 13H), 3.44 (s, 3H), 3.24 (s, 3H), 3.22 (s, 3H), 3.19 (s, 3H), 3.12 (s, 3H), 2.68 (s, 3H), 2.67 (s, 3H), 2.50-1.50 (m, 10H), 2.02 (s, 3H), 1.40-0.82 (m, 58H); ESI MS m/z 1383 [C₆₅H₁₁₂IN₁₁O₁₃+H]⁺.

### Example 30

### Preparation of Cyclosporin Vinyl Iodide

A mixture of the acetate of cyclosporin vinyl iodide from Example 29 (27 mg, 0.02 mmol), potassium carbonate (40 mg, 0.29 mmol) and methanol (1 mL) was stirred at room temperature for 4 h, and then diluted with ethyl acetate, washed with water and brine, dried over sodium sulfate, filtered, and concentrated in vacuo. The residue was purified by semi-preparative HPLC to afford cyclosporin vinyl iodide (14 mg, 54%) as a white solid: ¹H NMR (300 MHz, CDCl₃) δ 7.95 (d, J=9.7 Hz, 1H), 7.64 (d, J=7.5 Hz, 2H), 7.28 (d, J=7.3 Hz, 1H), 6.99 (dd, J=14.6, 10.8 Hz, 1H), 6.20-3.80 (m, 15H), 3.50 (s, 3H), 3.39 (s, 3H), 3.25 (s, 3H), 3.12 (s, 3H), 3.10 (s, 3H), 2.70 (s, 3H), 2.69 (s, 3H), 2.50-1.50 (m, 1H), 1.40-0.82 (m, 58H); ESI MS m/z 1340 [C₆₃H₁₁₀IN₁₁O₁₂+H]⁺; HPLC>99% (AUC), t_{R}=14.38 min.

### Example 31

### Preparation of Cyclosporin Fluoride

A 25 mL round bottom flask was charged with cyclosporin A (90 mg, 0.072 mmol), α,α,α-trifluorotoluene (5 mL), 3,3,3-trifluoropropene (200 mg, 2.08 mmol), and tricyclohexylphosphine[1,3-bis(2,4,6-trimethylphenyl)-4,5-dihydroimidazol-2-yl-ene][benzylidine]ruthenium(IV)dichloride (15 mg, 0.018 mmol). The atmosphere was maintained via a balloon filled with 3,3,3-trifluoropropene. The mixture was stirred at 50° C. for 72 h with 3,3,3-trifluoropropene refilled every 24 h. After cooling down to room temperature, solvent was removed in vacuo, the residue was pre-purified by column chromatography (silical gel, 6:1 EtOAc/CH₃CN) to give 40 mg of light brown solid. The obtained solid was purified via semi-preparative HPLC to give cyclosporin fluoride (12 mg, 12.7%) as a white solid: ¹H NMR (CDCl₃, 500 MHz) δ 8.25 (d, J=9.8 Hz, 1H), 8.01 (d, J=7.4 Hz, 1H), 7.87 (d, J=7.7 Hz, 1H), 7.56 (d, J=7.3 Hz, 1H), 6.62 (m, 1H), 5.93-5.84 (m, 2H), 5.73 (d, J=6.0 Hz, 1H), 5.51-5.47 (m, 1H), 5.48-5.30 (m, 4H), 5.14 (dd, J=6.3, 3.1 Hz, 1H), 5.03 (t, J=7.5 Hz, 1H), 4.95 (d, J=14.6 Hz, 1H), 4.86 (t, J=8.4 Hz, 1H), 4.73 (t, J=8.5 Hz, 1H), 3.97 (t, J=6.7 Hz, 1H), 3.66 (s, 3H), 3.52 (s, 3H), 3.38 (s, 3H), 3.37 (m, 2H), 3.24 (s, 3H), 3.20 (m, 2H), 2.85 (s, 3H), 2.81 (s, 3H), 2.70 (m, 1H), 2.48 (m, 1H), 2.32-2.05 (m, 7H), 1.90-1.65 (m, 9H), 1.68-0.78 (m, 49H); ¹⁹F NMR (CDCl₃, 282 MHz) δ −64.1, −76.3; ESI MS m/z 1256 [C₆₂H₁₀₈F₃N₁₁O₁₂+H]⁺; HPLC 95.2% (AUC), t_{R}=16.65 min.

### Example 32

### Preparation of Cyclosporin Fluoride

A 25 mL round bottom flask was charged with cyclosporin A (100 mg, 0.08 mmol), dichloromethane (5 mL), 1H,1H,2H-heptafluoropent-1-ene (200 mg, 1.02 mmol), and tricyclohexylphosphine[1,3-bis(2,4,6-trimethylphenyl)-4,5-dihydroimidazol-2-yl-ene][benzylidine]ruthenium(IV)dichloride (16 mg, 0.019 mmol). The mixture was refluxed at 50° C. for 48 h. After cooling down to room temperature, solvent was removed in vacuo, the residue was pre-purified by column chromatography (silical gel, 6:1 EtOAc/CH₃CN) to give 40 mg light brown solid. The obtained solid was purified via semi-preparative HPLC to give cyclosporin fluoride (12 mg, 10.6%) as a white solid: ¹H NMR (CDCl₃, 500 MHz) δ 8.02 (d, J=9.9 Hz, 1H), 7.69 (d, J=7.7 Hz, 1H), 7.49 (d, J=8.1 Hz, 1H), 7.16 (d, J=7.8 Hz, 1H), 6.40 (m, 1H), 5.69 (dd, J=10.1, 4.2 Hz, 1H), 5.53 (d, J=6.0 Hz, 1H), 5.26 (dd, J=16.2, 6.9 Hz, 1H), 5.04-4.99 (m, 2H), 4.92 (dd, J=9.8, 6.0 Hz, 1H), 4.82 (t, J=7.4 Hz, 1H), 4.72 (d, J=14.0 Hz, 1H), 4.62 (t, J=9.5 Hz, 1H), 4.53 (t, J=7.4 Hz, 1H), 3.78 (t, J=8.5 Hz, 1H), 3.52 (s, 3H), 3.38 (s, 3H), 3.25 (s, 3H), 3.12 (s, 3H), 3.11(s, 3H), 2.72 (s, 3H), 2.69 (s, 3H), 2.40 (m, 1H), 2.20-1.50 (m, 1H), 1.48-0.67 (m, 59H); ¹⁹F NMR (CDCl₃, 282 MHz): 6-76.3, −80.8, −112.6, −128.3; ESI MS m/z 1357 [C₆₄H₁₀₈F₇N₁₁O₁₂+H]⁺; HPLC 97.7% (AUC), t_{R}=17.66 min.

### Example 33

### Preparation of Cyclosporin Fluoride

A 25 mL round bottom flask was charged with cyclosporin A (100 mg, 0.08 mmol), dichloromethane (4 mL), 1H,1H,2H-Perfluoro-1-hexene (200 mg, 1.37 mmol), and tricyclohexylphosphine[1,3-bis(2,4,6-trimethylphenyl)-4,5-dihydroimidazol-2-yl-ene][benzylidine]ruthenium(IV)dichloride (15 mg, 0.018 mmol). The mixture was refluxed under nitrogen at 50° C. for 20 h. After cooling down to room temperature, solvent was removed in vacuo, the residue was pre-purified by column chromatography (silical gel, 6:1 EtOAc/CH₃CN) to give 90 mg light brown solid. The obtained solid was purified via semi-preparative HPLC to give the target cyclosporin fluoride (22 mg, 18.8%) as white solid: ¹H NMR (CDCl₃, 500 MHz) δ 8.00 (d, J=9.9 Hz, 1H), 7.67 (d, J=7.7 Hz, 1H), 7.47 (d, J=8.1 Hz, 1H), 7.14 (d, J=7.8 Hz, 1H), 6.38 (m, 1H), 5.67 (dd, J=10.1, 4.2 Hz, 1H), 5.51 (d, J=6.0 Hz, 1H), 5.24 (dd, J=16.2, 6.9 Hz, 1H), 5.04-4.99 (m, 2H), 4.90 (dd, J=9.8, 6.0 Hz, 1H), 4.80 (t, J=7.4 Hz, 1H), 4.70 (d, J=14.0 Hz, 1H), 4.60 (t, J=9.5 Hz, 1H), 4.51 (t, J=7.4 Hz, 1H), 3.78 (t, J=8.5 Hz, 1H), 3.50 (s, 3H), 3.36 (s, 3H), 3.23 (s, 3H), 3.08 (s, 3H), 3.07 (s, 3H), 2.68 (s, 3H), 2.65 (s, 3H), 2.40 (m, 1H), 2.18-1.88 (m, 6H), 1.87-1.50 (m, 9H), 1.48-0.67 (m, 55H); ¹⁹F NMR (CDCl₃, 282 MHz) 6-76.3, −81.5, −112.0, −124.6, −126.2; ESI MS m/z 1406 [C₆₅H₁₀₈F₉N₁₁O₁₂+H]⁺; HPLC>99.1% (AUC), t_{R}=30.84 min.

### Example 34

### Preparation of Cyclosporin allylic Fluoride

A 25 mL round bottom flask was charged with cyclosporin A (100 mg, 0.083 mmol), allyl fluoride (50 mg, 0.83 mmol), and tricyclohexylphosphine[1,3-bis(2,4,6-trimethylphenyl)-4,5-dihydroimidazol-2-yl-ene][benzylidine]ruthenium(IV) dichloride (34 mg, 0.04 mmol) and 5 mL of CH₂Cl₂. The mixture was refluxed under nitrogen overnight. After cooling down to room temperature, solvent was removed in vacuo. The residue was pre-purified by column chromatography (silica gel, 1:1 hexane/acetone) then purified via semi-preparative HPLC to give cyclosporin allylic fluoride (52 mg, 51.2%) as a white solid: ¹H NMR (CDCl₃, 300 MHz) δ 8.04 (d, J=9.6 Hz, 1H), 7.68 (d, J=7.3 Hz, 1H), 7.57 (d, J=8.2 Hz, 1H), 7.23 (d, J=7.8 Hz, 1H), 5.80-5.62 (m, 6H), 5.50 (d, J=6.2 Hz, 1H), 5.30 (dd, J=6.9, 3.5 Hz, 1H), 5.11-4.93 (m, 5H), 4.84 (d, J=5.6 Hz, 2H), 4.78 (d, J=15.6 Hz, 1H), 4.68 (d, J=7.1 Hz, 1H), 4.62 (d, J=8.9 Hz, 1H), 4.51 (t, J=7.3 Hz, 1H), 3.81 (t, J=6.4 Hz, 1H), 3.50 (s, 3H), 3.40 (s, 3H), 3.25 (s, 3H), 3.12 (s, 3H), 3.11 (s, 3H), 2.71 (s, 3H), 2.69 (s, 3H), 2.20-0.62 (m, 65H); ESI MS m/z 1221 [C₆₂H₁₁₀FN₁₁O₁₂+H]⁺; HPLC 94.1% (AUC), t_{R}=15.0 min.

### Example 35

### Preparation of the Acetate of Cyclosporin Allylic Chloride

Acetyl-protected cyclosporine A (100 mg, 0.08 mmol) was dissolved in 5 mL of CH₂Cl₂, and then allyl chloride (61 mg, 0.8 mmol) and Grubbs' catalyst 3^{rd} generation (25 mg, 0.04 mmol) were added. The mixture was refluxed under N₂ for 48 h. After that, the solvent was removed in vacuo, and the residue was purified via silica gel column (EtOAc) to give the acetate of cyclosporin allylic chloride (97 mg, yield 94%) as a light yellow solid: ¹H NMR (CDCl₃, 300 MHz) δ 8.54 (d, J=9.6 Hz, 1H), 8.04 (d, J=6.9 Hz, 1H), 7.70 (d, J=9.0 Hz, 1H), 7.51 (d, J=7.8 Hz, 1H), 5.68 (dd, J=1.1, 3.9 Hz, 1H), 5.53-5.47 (m, 6H), 5.17 (d, J=7.8 Hz, 2H), 4.97 (d, J=11.1 Hz, 3H), 4.84 (t, J=7.2 Hz, 1H), 4.75 (t, J=9.9 Hz, 1H), 4.64 (d, J=13.8 Hz, 1H), 4.43 (t, J=6.9 Hz, 1H), 3.98 (d, J=4.5 Hz, 2H), 3.45 (s, 3H), 3.25 (s, 3H), 3.24 (s, 3H), 3.20 (s, 3H), 3.16 (m, 2H), 3.11 (s, 3H), 2.67 (s, 3H), 2.66 (s, 3H), 2.41 (m, 1H), 2.22-0.78 (m, 66H); ESI MS m/z 1279 [C₆₄H₁₁₂ClN₁₁O₁₃+H]⁺.

### Example 36

### Preparation of Cyclosporin Allylic Chloride

The acetate of cyclosporin allylic chloride from Example 35 (30 mg, 0.023 mmol) was dissolved in 2 mL of methanol, and then K₂CO₃ (190 mg, 1.127 mmol) was added. The mixture was stirred at room temperature for 1.5 h, and then diluted with 100 mL of EtOAc, washed with H₂O (10 mL), brine (10 mL), dried over Na₂SO₄, solvent was removed in vacuo, the residue was purified via semi-preparative HPLC to give cyclosporin allylic chloride (15 mg, 51%) as a white solid:

¹H NMR (CDCl₃, 500 MHz) δ 8.05 (d, J=10.1 Hz, 1H), 7.65 (d, J=7.3 Hz, 1H), 7.49 (d, J=8.1 Hz, 1H), 7.16 (d, J=7.8 Hz, 1H), 5.70 (m, 2H), 5.57 (t, J=7.8 Hz, 1H), 5.49 (d, J=6.4 Hz, 1H), 5.28 (m, 1H), 5.12-4.94 (m, 4H), 4.82 (q, J=7.0 Hz, 1H), 4.72 (d, J=13.9 Hz, 1H), 4.65 (t, J=7.0 Hz, 1H), 4.53 (t, J=7.2 Hz, 1H), 4.02 (d, J=7.0 Hz, 1H), 3.82 (t, J=6.6 Hz, 1H), 3.51 (s, 3H), 3.40 (s, 3H), 3.26 (s, 3H), 3.11 (s, 6H), 2.70 (s, 3H), 2.68 (s, 3H), 2.48 (m, 2H), 2.22-0.62 (m, 68H); ESI MS m/z 1237 [C₆₂H₁₁₀ClN₁₁O₁₂+H]⁺; HPLC 97.9% (AUC), t_{R}=15.5 min.

### Example 37

### Preparation of Cyclosporin Amine

The acetate of cyclosporin allylic chloride from Example 35 (35 mg, 0.027 mmol) was mixed with dimethyl amine in THF (1.0 M, 6.0 mL, 6.0 mmol). The mixture was stirred under nitrogen at room temperature overnight. After that, the solvent was removed in vacuo to give the crude acetyl cyclosporin amine (40 mg). The crude product (40 mg, 0.03 mmol) was dissolved in 3 mL of methanol, and then K₂CO₃ (215 mg, 1.55 mmol) was added. The mixture was stirred at room temperature for 4 h, then diluted with 100 mL of EtOAc, washed with brine (3×10 mL), dried over Na₂SO₄. Solvent was removed in vacuo, and the residue was purified via semi-preparative HPLC to give cyclosporin amine (10.1 mg, 26%) as a white solid: ¹H NMR (CDCl₃, 300 MHz) δ 8.18 (d, J=9.6 Hz, 1H), 7.75 (d, J=7.5 Hz, 1H), 7.64 (d, J=8.1 Hz, 1H), 7.24 (d, J=8.2 Hz, 1H), 5.92 (m, 1H), 5.69 (dd, J=11.2, 3.7 Hz, 2H), 5.50 (m, 4H), 5.23 (dd, J=11.6, 3.3 Hz, 2H), 5.11-4.96 (m, 8H), 4.85 (t, J=7.2 Hz, 2H), 4.69 (d, J=13.6 Hz, 1H), 4.62 (t, J=8.8 Hz, 1H), 4.52 (t, J=7.4 Hz, 1H), 3.59 (t, J=6.4 Hz, 1H), 3.49 (s, 3H), 3.39 (s, 3H), 3.24 (s, 3H), 3.14 (s, 3H), 3.11 (s, 3H), 2.78 (dd, J=9.1, 3.1 Hz, 8H), 2.69 (s, 3H), 2.68 (s, 3H), 2.50-0.62 (m, 60H); ESI MS m/z 1246 [C₆₄H₁₁₆N₁₂O₁₂+H]⁺; HPLC 97.3% (AUC), t_{R}_{=13.5} min.

### Example 38

### Preparation of Cyclosporin Pyrrolidine

The acetate of cyclosporin allylic chloride from Example 35 (13 mg, 0.01 mmol) and pyrrolidine (7 mg, 0.1 mmol) were dissolved in 2 mL of CH₂Cl₂, and the mixture was stirred at room temperature under nitrogen for 48 h. The solvent was removed in vacuo to give the crude acetyl pyrrolidine compound (13 mg). The crude product (13 mg, 0.01 mmol) was dissolved in 2 mL of methanol, and then K₂CO₃ (50 mg, 0.36 mmol) was added. The mixture was stirred at room temperature for 48 h, then diluted with 100 mL of EtOAc, washed with brine (3×10 mL), dried over Na₂SO₄. Solvent was removed in vacuo, and the residue was purified via semi-preparative HPLC to give cyclosporin pyrrolidine (3 mg, 23%) as a white solid: ¹H NMR (CDCl₃, 500 MHz) δ 8.20 (d, J=9.6 Hz, 1H), 7.74 (d, J=7.2 Hz, 1H), 7.60 (d, J=8.2 Hz, 1H), 7.22 (d, J=7.7 Hz, 1H), 5.82 (m, 1H), 5.70 (dd, J=11.0, 3.9 Hz, 1H), 5.52 (m, 1H), 5.45 (d, J=6.3 Hz, 1H), 5.23 (dd, J=12.0, 4.8 Hz, 1H), 5.15-4.94 (m, 6H), 4.83 (t, J=7.3 Hz, 1H), 4.70 (d, J=13.8 Hz, 1H), 4.62 (t, J=8.8 Hz, 1H), 4.52 (t, J=7.4 Hz, 1H), 3.90 (m, 1H), 3.72 (m, 2H), 3.50 (s, 3H), 3.41 (s, 3H), 3.26 (s, 3H), 3.25 (m, 2H), 3.15 (s, 3H), 3.14 (s, 3H), 3.02-2.85 (m, 10H), 2.69 (s, 3H), 2.67 (s, 3H), 2.42 (m, 2H), 2.22-0.62 (m, 61H); ESI MS m/z 1272 [C₆₆H₁₁₈N₁₂O₁₂+H]⁺; HPLC 96.6% (AUC), t_{R}=14.4 min.

### Example 39

### Preparation of Cyclosporin Acetamide

The acetate of cyclosporin allylic chloride from Example 35 (30 mg, 0.023 mmol) was mixed with methylamine in THF (2.0 M, 4.0 mL, 8.0 mmol). The mixture was stirred at room temperature under nitrogen overnight. After that, the solvent was removed in vacuo to give the crude acetyl cyclosporin methylamine (30 mg). The methylamine (30 mg, 0.024 mmol) was dissolved in 4 mL of CH₂Cl₂, and then Ac₂O (48 mg, 0.47 mmol), pyridine (37 mg, 0.47 mmol) and DMAP (3 mg, 0.024 mmol) were added. The mixture was stirred under nitrogen at room temperature overnight, then diluted with 100 mL of EtOAc, washed with brine (3×10 mL), and dried over Na₂SO₄. Solvents were removed in vacuo, and the residue was purified via semi-preparative HPLC to give cyclosporin acetamide (12 mg, 39%) as a white solid. ¹H NMR (CDCl₃, 300 MHz) δ 8.49 (d, J=8.1 Hz, 1H), 8.04 (d, J=6.9 Hz, 1H), 7.82 (t, J=6.9 Hz, 1H), 7.55 (d, J=6.0 Hz, 1H), 5.68 (dd, J=12.2, 4.0 Hz, 1H), 5.28 (m, 3H), 5.17 (t, J=6.0 Hz, 2H), 4.96 (d, J=11.1 Hz, 3H), 4.84 (t, J=7.2 Hz, 2H), 4.68 (d, J=14.1 Hz, 3H), 4.44 (t, J=7.5 Hz, 1H), 3.88 (d, J=5.1 Hz, 1H), 3.48 (s, 3H), 3.25 (s, 3H), 3.22 (s, 3H), 3.20 (s, 3H), 3.10 (s, 3H), 2.93 (d, J=6.9 Hz, 4H), 2.68 (s, 3H), 2.67 (s, 3H), 2.24-2.18 (m, 7H), 2.01 (s, 3H), 2.12-0.62 (m, 63H); ESI MS m/z 1315 [C₆₇H₁₁₈N₁₂O₁₄+H]⁺.

### Example 40

### Preparation of Cyclosporin Amide

Cyclosporin amide from Example 39 (12 mg, 0.009 mmol) was dissolved in 2 mL of methanol, and then K₂CO₃ (63 mg, 0.456 mmol) was added. The mixture was stirred at room temperature for 4 h, then diluted with 100 mL of EtOAc, washed with brine (3×10 mL), dried over Na₂SO₄. Solvents were removed in vacuo, and the residue was purified via semi-preparative HPLC to give cyclosporin amide (4.5 mg, 38%) as a white solid: ¹H NMR (CD₂Cl₂, 500 MHz) δ 8.00 (d, J=10.5 Hz, 1H), 7.59 (d, J=4.5 Hz, 2H), 7.25 (s, 1H), 5.69 (dd, J=10.5, 4.0 Hz, 1H), 5.63 (m, 1H), 5.47 (d, J=4.5 Hz, 2H), 5.28 (m, 1H), 5.07 (d, J=6.5 Hz, 3H), 5.00 (t, J=7.0 Hz, 2H), 4.81 (t, J=7.0 Hz, 2H), 4.70 (d, J=14.0 Hz, 1H), 4.62 (t, J=9.0 Hz, 1H), 4.47 (t, J=7.5 Hz, 1H), 4.00-3.87 (m, 3H), 3.80 (t, J=6.5 Hz, 1H), 3.45 (s, 3H), 3.36 (s, 3H), 3.22 (s, 3H), 3.10 (s, 6H), 2.93 (d, J=9.0 Hz, 6H), 2.70 (s, 3H), 2.66 (s, 3H), 2.45 (m, 2H), 2.17 (s, 3H), 2.12-0.62 (m, 6H); ESI MS m/z 1274 [C₆₅H₁₁₆N₁₂O₁₃+H]⁺; HPLC 93.7% (AUC), t_{R}=14.3 min.

### Example 41

### Preparation of Cyclosporin Piperidine

A solution of the acetate of cyclosporin allylic chloride from Example 35 (50 mg, 0.04 mmol) and piperidine (33 mg, 0.4 mmol) in methylene chloride (3 mL) was stirred overnight at room temperature under N₂ atmosphere. Mixture was diluted with ether and extracted with 1 N HCl. Aqueous layer was neutralized with saturated sodium bicarbonate solution, extracted with ethyl acetate, washed with brine, dried over sodium sulfate, and concentrated in vacuo to afford the crude cyclosporin acetate (12 mg) as an off-white solid. A solution of the crude acetate (12 mg, 0.009 mmol) and potassium carbonate (13 mg, 0.099 mmol) in methanol (1 mL) was stirred overnight at room temperature under N₂ atmosphere. Mixture was diluted with ethyl acetate, washed with saturated sodium bicarbonate solution and brine, dried over sodium sulfate, and concentrated in vacuo. The crude product was purified by semi-preparative HPLC to afford cyclosporin piperidine (8.3 mg, 17%) as an off-white solid: ¹H NMR (300 MHz, CD₂Cl₂) δ 8.15 (d, J=9.7 Hz, 1H), 7.70 (d, J=7.3 Hz, 1H), 7.61 (d, J=8.3 Hz, 1H), 7.25 (d, J=7.8 Hz, 1H), 5.71 (d, J=6.8 Hz, 1H), 5.44 (d, J=7.5 Hz, 1H), 5.40-4.90 (m, 6H), 4.84 (t, J=4.38 Hz, 2H), 4.79 (s, 1H), 4.69 (dd, J=17.4, 14.0 Hz, 2H), 4.47 (t, J=14.3 Hz, 2H), 4.16 (d, J=10.7 Hz, 2H), 3.47 (s, 3H), 3.41 (s, 3H), 3.39 (s, 3H), 3.26 (s, 3H), 3.15 (s, 3H), 2.71 (s, 3H), 2.69 (s, 3H), 1.51 (d, J=7.3 Hz, 2H), 1.40-1.20 (m, 22H), 1.40-0.80 (m, 54H); ESI MS m/z 1286 [C₆₇H₁₂₀N₁₂O₁₂+H]⁺; HPLC 96.4% (AUC), t_{R}=14.69 min.

### Example 42

### Preparation of Cyclosporin Morpholine

A solution of the acetate of cyclosporin allylic chloride from Example 35 (75 mg, 0.058 mmol) and morpholine (51 mg, 0.58 mmol) in methylene chloride (3 mL) was stirred overnight at room temperature under N₂ atmosphere. The mixture was concentrated in vacuo. The residue and potassium carbonate (114 mg, 0.83 mmol) were dissolved in methanol (3 mL) and allowed to stir overnight at room temperature under N₂ atmosphere. The mixture was diluted with ethyl acetate, washed with saturated sodium bicarbonate solution and brine, dried over sodium sulfate, and concentrated in vacuo. The crude product was purified by semi-preparative HPLC to afford cyclosporin morpholine (28.2 mg, 29%) as a pale yellow solid: ¹H NMR (300 MHz, CD₂Cl₂) δ 7.97 (d, J=9.6 Hz, 1H), 7.57 (d, J=6.9 Hz, 1H), 7.44 (d, J=7.4 Hz, 1H), 7.15 (d, J=7.6 Hz, 1H), 5.67 (d, J=6.8 Hz, 1H), 5.44 (d, J=6.2 Hz, 1H), 5.10-4.90 (m, 1H), 4.79 (t, J=14.5 Hz, 2H), 4.72 (s, 1H), 4.63 (dd, J=14.7, 6.4 Hz, 2H), 4.42 (t, J=14.2 Hz, 2H), 3.45 (s, 3H), 3.36 (s, 3H), 3.21 (s, 3H), 3.09 (s, 3H), 3.08 (s, 3H), 2.68 (s, 3H), 2.56 (s, 3H), 2.50-1.40 (m, 19H), 1.27-0.70 (m, 54H); ESI MS m/z 1288 [C₆₆H₁₁₈N₁₂O₁₃+H]⁺; HPLC>99% (AUC), t_{R}=15.16 min.

### Example 43

### Preparation of Cyclosporin Thiomorpholine

A solution of the acetate of cyclosporin allylic chloride from Example 35 (60 mg, 0.047 mmol) and thiomorpholine (48 mg, 0.47 mmol) in methylene chloride (3 mL) was stirred overnight at room temperature under N₂ atmosphere. The mixture was concentrated in vacuo. The residue and potassium carbonate (102 mg, 0.740 mmol) were dissolved in methanol (3 mL) and allowed to stir overnight at room temperature under N₂ atmosphere. The mixture was diluted with ethyl acetate, washed with saturated sodium bicarbonate solution and brine, dried over sodium sulfate, and concentrated in vacuo. The crude product was purified by semi-preparative HPLC to afford cyclosporine thiomorpholine (28.8 mg, 33%) as a white solid: ¹H NMR (300 MHz, CD₂Cl₂) δ 7.96 (d, J=9.7 Hz, 1H), 7.59 (d, J=7.2 Hz, 1H), 7.44 (d, J=7.4 Hz, 1H), 7.15 (d, J=7.7 Hz, 1H), 5.67 (dd, J=10.9, 4.1 Hz, 1H), 5.05-5.48 (m, 9H), 5.43 (d, J=6.6 Hz, 2H), 5.28 (d, J=3.8 Hz, 1H), 5.24 (d, J=3.8 Hz, 1H), 5.15-4.89 (m, 5H), 4.79 (t, J=14.5 Hz, 2H), 4.72 (s, 1H), 4.64 (dd, J=14.8, 5.8 Hz, 2H), 4.23 (t, J=14.5 Hz, 2H), 3.44 (s, 3H), 3.35 (s, 3H), 3.21(s, 3H), 3.09 (s, 3H), 3.08 (s, 3H), 2.68 (s, 3H), 2.65 (s, 3H), 2.50-1.40 (m, 15H), 1.27-0.70 (m, 54H): ESI MS m/z 1304 [C₆₆H₁₁₈N₁₂O₁₂S+H]⁺; HPLC>99% (AUC), t_{R}=13.00 min.

### Example 44

### Preparation of Cyclosporin Methylpiperazine

A solution of the acetate of cyclosporin allylic chloride from Example 35 (75 mg, 0.058 mmol) and methylpiperazine (58 mg, 0.58 mmol) in methylene chloride (3 mL) was stirred overnight at room temperature under N₂ atmosphere. The mixture was concentrated in vacuo. The residue and potassium carbonate (102 mg, 0.740 mmol) were dissolved in methanol (3 mL) and allowed to stir overnight at room temperature under N₂ atmosphere. The mixture was diluted with ethyl acetate, washed with saturated sodium bicarbonate solution and brine, dried over sodium sulfate, and concentrated in vacuo. The crude product was purified by semi-preparative HPLC to afford cyclosporin methylpiperazine (36.2 mg, 42%) as an off-white solid: ¹H NMR (300 MHz, CD₂Cl₂) δ 7.95 (d, J=9.6 Hz, 1H), 7.58 (d, J=7.1 Hz, 1H), 7.42 (d, J=8.2 Hz, 1H), 7.15 (d, J=7.5 Hz, 1H), 5.67 (d, J=7.1 Hz, 1H), 5.43 (d, J=6.3 Hz, 1H), 5.15-4.90 (m, 10H), 4.79 (t, J=14.6 Hz, 2H), 4.72 (s, 1H), 4.64 (dd, J=14.3, 5.3 Hz, 2H), 4.42 (t, J=14.4 Hz, 2H), 3.45 (s, 3H), 3.36 (s, 3H), 3.21 (s, 3H), 3.09 (s, 3H), 3.07 (s, 3H), 2.69 (s, 3H), 2.65 (s, 3H), 2.28 (s, 3H), 2.15-1.40 (m, 19H), 1.27-0.70 (m, 55H); ESI MS m/z 1301 [C₆₇H₁₂₁N₁₃O₁₂+H]⁺; HPLC>99% (AUC), t_{R}=15.89 min.

### Example 45

### Preparation of the Acetate of Cyclosporin ene-ene-yne

Zinc chloride (1.0 M in ether, 0.62 mL, 0.62 mmol) was added to a solution of 1-propynylmagnesium bromide (0.5 M in THF, 1.24 mL, 0.62 mmol) in THF (1 mL) at 0° C. and the mixture was stirred under nitrogen for 10 min. The ice bath was removed and the mixture was warmed to room temperature. The acetate of cyclosporin vinyl iodide from Example 29 (85 mg, 0.062 mmol) in THF (2 mL) was added, followed by addition of bis(triphenylphosphine)dichloropalladium(II) (4.3 mg, 0.0062 mmol). The mixture was stirred at room temperature for 1 h, quenched with saturated aqueous ammonium chloride, extracted with ethyl acetate. The combined organic layers were washed with brine, dried over sodium sulfate, filtered and concentrated in vacuo. The residue was purified by semi-preparative HPLC to afford the acetate of cyclosporin ene-ene-yne (17 mg, 22%) as a yellow oil: ¹H NMR (300 MHz, CDCl₃) δ 8.56 (d, J=9.7 Hz, 1H), 8.05 (d, J=6.8 Hz, 1H), 7.64 (d, J=9.2 Hz, 1H), 7.50 (d, J=7.6 Hz, 1H), 6.45 (dd, J=15.5, 10.8 Hz, 1H), 5.90 (dd, J=15.3, 10.8 Hz, 1H), 5.70-4.40 (m, 14H), 3.44 (s, 3H), 3.25 (s, 3H), 3.20 (s, 6H), 3.10 (s, 3H), 2.68 (s, 3H), 2.66 (s, 3H), 2.50-1.50 (m, 10H), 2.01 (s, 3H), 1.96 (d, J=2.0 Hz, 3H), 1.40-0.82 (m, 58H); ESI MS m/z 1295 [C₆₈H₁₁₅N₁₁O₁₃+H]⁺.

### Example 46

### Preparation of Cyclosporin ene-ene-yne

A mixture of the acetate of cyclosporin ene-ene-yne from Example 45 (17 mg, 0.013 mmol), potassium carbonate (30 mg, 0.22 mmol) and methanol (1 mL) was stirred at room temperature overnight, and then diluted with ethyl acetate, washed with water and brine, dried over sodium sulfate, filtered, and concentrated in vacuo. The residue was purified by semi-preparative HPLC to afford cyclosporin ene-ene-yne (6 mg, 36%) as a white solid: ¹H NMR (300 MHz, CDCl₃) δ 7.98 (d, J=9.4 Hz, 1H), 7.65 (d, J=7.4 Hz, 1H), 7.52 (d, J=8.2 Hz, 1H), 7.19 (d, J=7.8 Hz, 1H), 6.47 (dd, J=15.5, 10.8 Hz, 1H), 5.99 (dd, J=15.1, 10.8 Hz, 1H), 5.78-3.75 (m, 14H), 3.51 (s, 3H), 3.39 (s, 3H), 3.25 (s, 3H), 3.11 (s, 3H), 3.10 (s, 3H), 2.71 (s, 3H), 2.69 (s, 3H), 2.50-1.50 (m, 1H), 1.96 (d, J=2.0 Hz, 3H), 1.40-0.82 (m, 58H); ESI MS m/z 1252 [C₆₆H₁₁₃N₁₁O₁₂+H]⁺; HPLC>99% (AUC), t_{R}=14.09 min.

### Example 47

### Preparation of the Acetate of Cyclosporin ene-ene-yne

A mixture of the acetate of cyclosporin vinyl iodide from Example 29 (250 mg, 0.180 mmol), 3-butyn-2-ol (0.13 mL, 1.8 mmol), bis(triphenylphosphine)dichloropalladium(II) (13 mg, 0.018 mmol), copper(I) iodide (7 mg, 0.036 mmol) and triethylamine (2 mL) was stirred under nitrogen at room temperature for 4 h. The mixture was diluted with ethyl acetate, filtered through a pad of silica gel, washed with ethyl acetate. The filtrate was concentrated in vacuo and the residue was purified by semi-preparative HPLC to afford the acetate of cyclosporin ene-ene-yne (166 mg, 70%) as a white solid: ¹H NMR (300 MHz, CDCl₃) δ 8.53 (d, J=9.5 Hz, 1H), 8.05 (d, J=7.0 Hz, 1H), 7.72 (d, J=9.0 Hz, 1H), 7.53 (d, J=7.6 Hz, 1H), 6.50 (dd, J=15.5, 10.8 Hz, 1H), 5.93 (dd, J=15.0, 10.8 Hz, 1H), 5.75-4.40 (m, 15H), 3.44 (s, 3H), 3.25 (s, 3H), 3.22 (s, 3H), 3.20 (s, 3H), 3.10 (s, 3H), 2.68 (s, 3H), 2.67 (s, 3H), 2.50-1.50 (m, 10H), 2.02 (s, 3H), 1.40-0.82 (m, 62H); ESI MS m/z 1324 [C₆₉H₁₁₇N₁₁O₁₄+H]⁺.

### Example 48

### Preparation of Cyclosporin ene-ene-yne

A mixture of the acetate of cyclosporin ene-ene-yne from Example 47 (11 mg, 0.008 mmol), potassium carbonate (30 mg, 0.22 mmol) and methanol (1 mL) was stirred at room temperature overnight, and then diluted with ethyl acetate, washed with water and brine, dried over sodium sulfate, filtered, and concentrated in vacuo. The residue was purified by semi-preparative HPLC to afford cyclosporin ene-ene-yne (5 mg, 45%) as a white solid: ¹H NMR (300 MHz, CDCl₃) δ 7.79 (t, J=9.5 Hz, 1H), 7.56 (d, J=8.0 Hz, 1H), 7.48 (d, J=9.0 Hz, 1H), 7.18 (d, J=7.6 Hz, 1H), 6.50 (dd, J=15.5, 10.8 Hz, 1H), 6.02 (dd, J=15.2, 10.8 Hz, 1H), 5.75-3.75 (m, 15H), 3.51 (s, 3H), 3.38 (s, 3H), 3.24 (s, 3H), 3.11 (s, 3H), 3.09 (s, 3H), 2.70 (s, 3H), 2.69 (s, 3H), 2.50-1.50 (m, 1H), 1.40-0.82 (m, 62H); ESI MS m/z 1282 [C₆₇H₁₁₅N₁₁O₁₃+H]⁺; HPLC>99% (AUC), t_{R}=13.42 min.

### Example 49

### Preparation of the Acetate of Cyclosporin ene-ene-yne-ene

A mixture of the crude acetate of cyclosporin ene-ene-yne from Example 47 (136 mg, 0.10 mmol), Burgess reagent (119 mg, 0.50 mmol) and benzene (2 mL) was heated at 70° C. for 5 h, and then cooled to room temperature, diluted with ether, washed with water and brine, dried over sodium sulfate, filtered, and concentrated in vacuo. The residue was purified by semi-preparative HPLC to afford the desired acetate of cyclosporin ene-ene-yne-ene (10 mg, 7%) as a white solid: ¹H NMR (300 MHz, CDCl₃) δ 8.53 (d, J=9.8 Hz, 1H), 8.05 (d, J=6.8 Hz, 1H), 7.76 (d, J=9.0 Hz, 1H), 7.56 (d, J=7.6 Hz, 1H), 6.57 (dd, J=14.5, 11.0 Hz, 1H), 5.93 (dd, J=14.3, 10.8 Hz, 1H), 5.76-4.40 (m, 17H), 3.44 (s, 3H), 3.26 (s, 3H), 3.23 (s, 3H), 3.20 (s, 3H), 3.11 (s, 3H), 2.67 (s, 3H), 2.66 (s, 3H), 2.50-1.50 (m, 10H), 2.02 (s, 3H), 1.40-0.82 (m, 58H); ESI MS m/z 1307 [C₆₉H₁₁₅N₁₁O₁₃+H]⁺.

### Example 50

### Preparation of Cyclosporin ene-ene-yne-ene

A mixture of the acetate of cyclosporin ene-ene-yne-ene from Example 49 (10 mg, 0.008 mmol), potassium carbonate (30 mg, 0.22 mmol) and methanol (1 mL) was stirred at room temperature for 8 h, and then diluted with ethyl acetate, washed with water and brine, dried over sodium sulfate, filtered, and concentrated in vacuo. The residue was purified by semi-preparative HPLC to afford cyclosporin ene-ene-yne-ene (3 mg, 30%) as a white solid: ¹H NMR (300 MHz, CDCl₃) δ 8.01 (d, J=10.0 Hz, 1H), 7.63 (d, J=7.0 Hz, 1H), 7.48 (d, J=9.0 Hz, 1H), 7.16 (d, J=8.5 Hz, 1H), 6.57 (dd, J=15.5, 10.5 Hz, 1H), 6.03 (dd, J=14.5, 10.5 Hz, 1H), 5.93 (ddd, J=17.5, 11.0, 2.0 Hz, 1H), 5.72-3.75 (m, 16H), 3.51 (s, 3H), 3.39 (s, 3H), 3.26 (s, 3H), 3.11 (s, 6H), 2.70 (s, 3H), 2.68 (s, 3H), 2.50-1.50 (m, 1H), 1.40-0.82 (m, 58H); ESI MS m/z 1265 [C₆₇H₁₁₃N₁₁O₁₂+H]⁺; HPLC 90.8% (AUC), t_{R}=14.55 min.

### Example 51

### Preparation of the Acetate of Cyclosporin ene-ene-yne

A mixture of the acetate of cyclosporin vinyl iodide from Example 29 (56 mg, 0.041 mmol), (trimethylsilyl)acetylene (0.056 mL, 0.41 mmol), bis(triphenylphosphine)dichloropalladium(II) (5.7 mg, 0.0082 mmol), copper(I) iodide (3.1 mg, 0.016 mmol), and triethylamine (1 mL) was stirred under nitrogen at room temperature for 1 h, and then diluted with ethyl acetate, washed with brine, dried over sodium sulfate, filtered, and concentrated in vacuo. The residue was purified by semi-preparative HPLC to afford the acetate of cyclosporin ene-ene-yne (34 mg, 64%) as a light yellow solid: ¹H NMR (300 MHz, CDCl₃) δ 8.55 (d, J=9.2 Hz, 1H), 8.04 (d, J=6.8 Hz, 1H), 7.68 (d, J=9.0 Hz, 1H), 7.53 (d, J=7.2 Hz, 1H), 6.55 (dd, J=15.5, 10.8 Hz, 1H), 5.90 (dd, J=14.8, 10.8 Hz, 1H), 5.70-4.40 (m, 14H), 3.43 (s, 3H), 3.24 (s, 3H), 3.22 (s, 3H), 3.19 (s, 3H), 3.10 (s, 3H), 2.67 (s, 3H), 2.65 (s, 3H), 2.50-1.50 (m, 10H), 2.00 (s, 3H), 1.40-0.82 (m, 58H), 0.17 (s, 9H); ESI MS m/z 1353 [C₇₀H₁₂₁N₁₁O₁₃Si+H]⁺.

### Example 52

### Preparation of Cyclosporin ene-ene-yne

A mixture of the acetate of cyclosporin ene-ene-yne from Example 51 (34 mg, 0.025 mmol), potassium carbonate (30 mg, 0.22 mmol) and methanol (1 mL) was stirred at room temperature overnight, and then diluted with ethyl acetate, washed with water and brine, dried over sodium sulfate, filtered, and concentrated in vacuo. The residue was purified by semi-preparative HPLC to afford cyclosporin ene-ene-yne (15 mg, 48%) as a white solid: ¹H NMR (300 MHz, CDCl₃) δ 7.98 (d, J=9.2 Hz, 1H), 7.65 (d, J=6.8 Hz, 1H), 7.61 (d, J=9.0 Hz, 1H), 7.25 (d, J=7.2 Hz, 1H), 6.64 (dd, J=15.6, 10.7 Hz, 1H), 6.02 (dd, J=14.9, 10.7 Hz, 1H), 5.80-3.75 (m, 14H), 3.50 (s, 3H), 3.39 (s, 3H), 3.25 (s, 3H), 3.12 (s, 3H), 3.10 (s, 3H), 2.98 (d, J=2.2 Hz, 1H), 2.70 (s, 3H), 2.69 (s, 3H), 2.50-1.50 (m, 1H), 1.4(00.82 (m, 58H); ESI MS m/z 1239 [C₆₅H₁₁₁N₁₁O₁₂+H]⁺; HPLC>99% (AUC), t_{R}=19.40 min.

### Example 53

### Preparation of the Acetate of cis-Cyclosporin Vinyl Iodide

Ethyl triphenylphosphonium iodide (203 mg, 0.49 mmol) was dissolved in THF (3 mL) and treated with n-BuLi (0.4 mL, 2.5 M in hexanes, 0.98 mmol) at room temperature under N₂ atmosphere. Reaction mixture was cooled to −78° C. and treated with a solution of 12 (109 mg, 0.43 mmol) in THF (2 mL). Mixture was stirred for 5 min and then warmed to −15° C. for 5 min. Next, the reaction was treated with sodium bis(trimethylsilyl)amide (0.4 mL, 1 M in THF, 0.41 mmol) and stirred for an additional 5 min. Acetyl cyclosporin aldehyde from Example 2 (500 mg, 0.4 mmol) was added to the reaction, stirred at −15° C. for 10 min, and then allowed to warm to room temperature. Reaction was poured into a saturated solution of NH₄Cl and extracted with ether. Organic layer washed with brine, dried over sodium sulfate, and concentrated in vacuo. The crude product was purified by semi-preparative HPLC to afford the acetate of the cis-cyclosporin vinyl iodide (13 mg, 2%) as an off-white solid: ¹H NMR (300 MHz, CDCl₃) δ 8.56 (d, J=9.6 Hz, 1H), 8.04 (d, J=6.8 Hz, 1H), 7.65 (d, J=9.1 Hz, 1H), 7.49 (d, J=7.7 Hz, 1H), 5.68 (dd, J=10.9, 3.8 Hz, 1H), 5.57 (s, 1H), 5.52 (d, J=6.1 Hz, 2H), 5.38 (dd, J=11.8, 3.5 Hz, 1H), 5.28-5.13 (m, 8H), 5.02 (d, J=10.5 Hz, 3H), 4.84 (t, J=7.3 Hz, 2H), 4.77 (t, J=9.5 Hz, 2H), 4.64 (d, J=13.8 Hz, 2H), 4.42 (t, J=7.0 Hz, 2H), 3.44 (s, 3H), 3.27 (s, 3H), 3.24 (s, 3H), 3.20 (s, 3H), 3.11 (s, 3H), 2.67 (s, 3H), 2.66 (s, 3H), 2.46 (s, 3H), 2.05 (s, 2H), 1.32 (d, J=7.1 Hz, 4H), 1.27 (d, J=7.1 Hz, 4H), 1.02-0.79 (m, 52H); ESI MS m/z 1371 [C₆₄H₁₁₂IN₁₁O₁₃+H]⁺.

### Example 54

### Preparation of cis-Cyclosporin Vinyl Iodide

A solution of the acetate of cis-cyclosporin vinyl iodide from Example 53 (13 mg, 0.009 mmol) in methanol (1 mL) was stirred at room temperature. Reaction mixture was treated with potassium carbonate (15 mg, 0.11 mmol) and was allowed to keep stirring under N₂ atmosphere overnight. Mixture was diluted with ethyl acetate, washed with saturated sodium bicarbonate solution and brine, dried over sodium sulfate, and concentrated in vacuo. The crude product was purified by semi-preparative HPLC to afford cis-cyclosporin vinyl iodide (6 mg, 47%) as a white solid: ¹H NMR (300 MHz, CDCl₃) δ 7.93 (d, J=9.8 Hz, 1H), 7.61 (d, J=7.2 Hz, 1H), 7.44 (d, J=8.3 Hz, 1H), 7.22 (d, J=8.0 Hz, 1H), 5.68 (dd, J=11.0, 4.2 Hz, 1H), 5.45 (d, J=6.7 Hz, 2H), 5.39-5.33 (m, 4H), 5.13-4.96 (m, 1H), 4.81 (t, J=7.5 Hz, 2H), 4.74-4.63 (m, 6H), 4.50 (t, J=7.2 Hz, 2H), 3.51 (s, 3H), 3.39 (s, 3H), 3.24 (s, 3H), 3.11 (s, 6H), 2.69 (s, 6H), 2.48 (s, 2H), 1.35 (d, J=7.2 Hz, 2H), 1.27-1.24 (m, 4H), 1.08-0.83 (m, 48H), 0.79 (d, J=6.6 Hz, 1H); ESI MS m/z 1329 [C₆₂H₁₁₀IN₁₁O₁₂+H]⁺; HPLC>99% (AUC), t_{R}=21.02 min.

### Example 55

### Preparation of the Acetate of Cyclosporin Oxime

Methoxyamine hydrochloride (4.3 mg, 0.052 mmol) was added to a solution of the acetate of cyclosporin α,β-unsaturated aldehyde from Example 24 (65 mg, 0.052 mmol) in pyridine (1 mL) at room temperature. The mixture was stirred under nitrogen for 1 h and then diluted with ether, washed with 0.2 N HCl and brine, dried over sodium sulfate, filtered, and concentrated in vacuo. The residue was purified by semi-preparative HPLC to afford the desired acetate of cyclosporin oxime (31 mg, 47%) as a white solid and a mixture of two isomers: ¹H NMR (300 MHz, CDCl₃) δ 8.54 (d, J=9.5 Hz, 1H), 8.04 (d, J=6.8 Hz, 1H), 7.70 (d, J=9.8 Hz, 2H), 7.51 (d, J=7.5 Hz, 1H), 6.51 (dd, J=15.5, 9.0 Hz, 1H), 6.00 (dd, J=15.5, 9.5 Hz, 1H), 5.70-4.41 (m, 12H), 3.86 (s, 3H), 3.45 (s, 3H), 3.25 (s, 3H), 3.22 (s, 3H), 3.20 (s, 3H), 3.11 (s, 3H), 2.68 (s, 3H), 2.66 (s, 3H), 2.50-1.50 (m, 10H), 2.02 (s, 3H), 1.40-0.82 (m, 58H); ESI MS m/z 1288 [C₆₅H₁₁₄N₁₂O₁₄+H]⁺.

### Example 56

### Preparation of Cyclosporin Oxime

A mixture of the acetate of cyclosporin oxime from Example 55 (24 mg, 0.019 mmol), potassium carbonate (30 mg, 0.22 mmol) and methanol (1 mL) was stirred at room temperature overnight, and then diluted with ethyl acetate, washed with water and brine, dried over sodium sulfate, filtered, and concentrated in vacuo. The residue was dissolved in methylene chloride and filtered through a microfilter (0.2 μm), and the filtrate was concentrated and dried under vacuum to afford cyclosporin oxime (21 mg, 90%) as a white solid: ¹H NMR (300 MHz, CDCl₃) δ 8.04 (d, J=9.5 Hz, 1H), 7.71 (d, J=9.5 Hz, 1H), 7.65 (d, J=7.5 Hz, 1H), 7.49 (d, J=8.5 Hz, 1H), 7.16 (d, J=7.8 Hz, 1H), 6.07 (dd, J=15.5, 9.5 Hz, 1H), 5.93 (ddd, J=15.4, 8.0, 6.0 Hz, 1H), 5.72-3.82 (m, 12H), 3.85 (s, 3H), 3.51 (s, 3H), 3.40 (s, 3H), 3.26 (s, 3H), 3.12 (s, 3H), 3.11 (s, 3H), 2.69 (s, 3H), 2.68 (s, 3H), 2.50-1.50 (m, 1H), 1.40-0.82 (m, 58H); ESI MS m/z 1246 [C₆₃H₁₁₂N₁₂O₁₃+H]⁺; HPLC>99% (AUC), t_{R}_{=13.31} min.

### Example 57

### Preparation of the Acetate of Cyclosporin Oxime

O-Ethylhydroxylamine hydrochloride (3.1 mg, 0.032 mmol) was added to a solution of the acetate of cyclosporin α,β-unsaturated aldehyde from Example 24 (40 mg, 0.032 mmol) in pyridine (0.5 mL) at room temperature. The mixture was stirred under nitrogen for 1 h and then diluted with ethyl acetate, washed with 1 N HCl and brine, dried over sodium sulfate, filtered, and concentrated in vacuo. The residue was purified by semi-preparative HPLC to afford the acetate of the desired cyclosporin oxime (8 mg, 20%) as a white solid and a mixture of two isomers: ¹H NMR (300 MHz, CDCl₃) δ 8.53 (d, J=9.5 Hz, 1H), 8.05 (d, J=6.8 Hz, 1H), 7.72 (d, J=9.8 Hz, 1H), 7.69 (d, J=9.8 Hz, 1H), 7.58 (d, J=7.5 Hz, 1H), 6.53 (dd, J=15.5, 9.0 Hz, 1H), 6.04 (dd, J=15.5, 9.1 Hz, 1H), 5.75-4.10 (m, 14H), 3.44 (s, 3H), 3.25 (s, 3H), 3.21 (s, 3H), 3.20 (s, 3H), 3.12 (s, 3H), 2.68 (s, 3H), 2.67 (s, 3H), 2.50-1.50 (m, 10H), 2.02 (s, 3H), 1.40-0.82 (m, 61H); ESI MS m/z 1302 [C₆₆H₁₁₆N₁₂O₁₄+H]⁺.

### Example 58

### Preparation of Cyclosporin Oxime

A mixture of the acetate of cyclosporin oxime from Example 57 (8 mg, 0.006 mmol), potassium carbonate (30 mg, 0.22 mmol) and methanol (1 mL) was stirred at room temperature for 4 h, and then diluted with ethyl acetate, washed with water and brine, dried over sodium sulfate, filtered, and concentrated in vacuo. The residue was dissolved in methylene chloride and filtered through a microfilter (0.2 μm), and the filtrate was concentrated and dried under vacuum to afford cyclosporin oxime (7 mg, 88%) as a white solid: ¹H NMR (500 MHz, CDCl₃) δ 8.04 (d, J=9.5 Hz, 1H), 7.72 (d, J=9.5 Hz, 1H), 7.63 (d, J=7.5 Hz, 1H), 7.49 (d, J=8.5 Hz, 1H), 7.16 (d, J=8.0 Hz, 1H), 6.07 (dd, J=15.5, 9.5 Hz, 1H), 5.92 (ddd, J=15.5, 8.5, 6.5 Hz, 1H), 5.71-3.82 (m, 14H), 3.51 (s, 3H), 3.40 (s, 3H), 3.26 (s, 3H), 3.11 (s, 3H), 3.11 (s, 3H), 2.69 (s, 3H), 2.68 (s, 3H), 2.50-1.50 (m, 1H), 1.40-0.82 (m, 61H); ESI MS m/z 1260 [C₆₄H₁₁₄N₁₂O₁₃+H]⁺; HPLC 95.0% (AUC), t_{R}=16.91 min.

### Example 59

### Preparation of the Acetate of Cyclosporin Oxime

O-Benzylhydroxylamine hydrochloride (5.1 mg, 0.032 mmol) was added to a solution of the acetate of cyclosporin α,β-unsaturated aldehyde from Example 24 (40 mg, 0.032 mmol) in pyridine (0.5 mL) at room temperature. The mixture was stirred under nitrogen for 1 h and then diluted with ethyl acetate, washed with 1 N HCl and brine, dried over sodium sulfate, filtered, and concentrated in vacuo. The residue was purified by semi-preparative HPLC to afford the desired acetate of cyclosporin oxime (7 mg, 16%) as a white solid and a mixture of two isomers: ¹H NMR (300 MHz, CDCl₃) δ 8.53 (d, J=9.5 Hz, 1H), 8.05 (d, J=6.8 Hz, 1H), 7.81 (d, J=9.3 Hz, 1H), 7.76 (d, J=9.9 Hz, 1H), 7.70-7.28 (m, 6H), 6.56 (dd, J=15.7, 9.4 Hz, 1H), 6.02 (dd, J=15.5, 10.2 Hz, 1H), 5.75-4.41 (m, 14H), 3.44 (s, 3H), 3.24 (s, 3H), 3.19 (s, 6H), 3.11 (s, 3H), 2.68 (s, 3H), 2.67 (s, 3H), 2.50-1.50 (m, 10H), 2.02 (s, 3H), 1.40-0.82 (m, 58H); ESI MS m/z 1364 [C₇₁H₁₁₈N₁₂O₁₄+H]⁺.

### Example 60

### Preparation of Cyclosporin Oxime

A mixture of the acetate of cyclosporin oxime from Example 59 (7 mg, 0.005 mmol), potassium carbonate (30 mg, 0.22 mmol) and methanol (1 mL) was stirred at room temperature for 4 h, and then diluted with ethyl acetate, washed with water and brine, dried over sodium sulfate, filtered and concentrated in vacuo. The residue was dissolved in methylene chloride and filtered through a microfilter (0.2 μm), and the filtrate was concentrated and dried under vacuum to afford cyclosporin oxime (6 mg, 86%) as a white solid: ¹H NMR (500 MHz, CDCl₃) δ 8.03 (d, J=9.5 Hz, 1H), 7.79 (d, J=10.0 Hz, 1H), 7.63 (d, J=7.5 Hz, 1H), 7.48 (d, J=8.0 Hz, 1H), 7.38-7.28 (m, 5H), 7.16 (d, J=8.0 Hz, 1H), 6.07 (dd, J=15.5, 10.0 Hz, 1H), 5.94 (ddd, J=15.5, 8.5, 6.5 Hz, 1H), 5.71-3.85 (m, 14H), 3.51 (s, 3H), 3.40 (s, 3H), 3.25 (s, 3H), 3.11 (s, 6H), 2.69 (s, 3H), 2.68 (s, 3H), 2.50-1.50 (m, 1H), 1.40-0.82 (m, 58H); ESI MS m/z 1322 [C₆₉H₁₁₆N₁₂O₁₃+H]⁺; HPLC 97.5% (AUC), t_{R}=20.40 min.

### Example 61

### Preparation of the Acetate of Cyclosporin Hydrazone

1,1-Dimethylhydrazine (2.4 μL, 0.032 mmol) was added to a solution of the acetate of cyclosporin α,β-unsaturated aldehyde from Example 24 (40 mg, 0.032 mmol) in methanol (1 mL) at room temperature. The mixture was stirred under nitrogen for 2 h and then diluted with ethyl acetate, washed with brine, dried over sodium sulfate, filtered and concentrated in vacuo. The residue was purified by semi-preparative HPLC to afford the desired acetate of cyclosporin hydrazone (15 mg, 36%) as a white solid and a mixture of two isomers: ¹H NMR (500 MHz, CDCl₃) δ 8.42 (d, J=9.5 Hz, 1H), 8.20 (d, J=9.5 Hz, 1H), 8.03 (d, J=7.0 Hz, 1H), 7.78 (d, J=9.0 Hz, 1H), 7.45 (d, J=7.5 Hz, 1H), 6.35 (dt, J=15.5, 8.0 Hz, 1H), 6.11 (dd, J=15.5, 9.5 Hz, 1H), 5.71-4.41 (m, 12H), 3.46 (s, 3H), 3.25 (s, 3H), 3.21 (s, 3H), 3.20 (s, 3H), 3.11 (s, 9H), 2.68 (s, 3H), 2.66 (s, 3H), 2.50-1.50 (m, 10H), 2.02 (s, 3H), 1.40-0.82 (m, 58H); ESI MS m/z 1301 [C₆₆H₁₁₇N₁₃O₁₃+H]⁺.

### Example 62

### Preparation of Cyclosporin Hydrazone

A mixture of the acetate of cyclosporin hydrazone from Example 61 (15 mg, 0.011 mmol), potassium carbonate (40 mg, 0.29 mmol) and methanol (1 mL) was stirred at room temperature for 6 h, and then diluted with ethyl acetate, washed with water and brine, dried over sodium sulfate, filtered and concentrated in vacuo. The residue was dissolved in methylene chloride and filtered through a microfilter (0.2 μm), and the filtrate was concentrated and dried under vacuum to afford cyclosporin hydrazone (12 mg, 82%) as a white solid: ¹H NMR (300 MHz, CDCl₃) δ 8.03 (d, J=9.8 Hz, 1H), 7.61 (d, J=7.4 Hz, 1H), 7.51 (d, J=8.3 Hz, 1H), 7.15 (d, J=7.9 Hz, 1H), 7.05 (d, J=8.9 Hz, 1H), 6.15 (dd, J=15.7, 8.9 Hz, 1H), 5.71-3.75 (m, 13H), 3.51 (s, 3H), 3.41 (s, 3H), 3.24 (s, 3H), 3.11 (s, 3H), 3.10 (s, 3H), 2.85 (s, 6H), 2.70 (s, 3H), 2.69 (s, 3H), 2.50-1.50 (m, 1H), 1.40-0.82 (m, 58H); ESI MS m/z 1259 [C₆₄H₁₁₅N₁₃O₁₂+H]⁺.

### Example 63

### Preparation of Cyclosporin Diol

To a mechanically stirred solution of diisopropylamine (2.6 mL, 18 mmol) in THF (50 mL) at −78° C. was added dropwise n-butyllithium (6.6 mL, 2.5 M in hexane, 17 mmol), then the mixture was stirred for 0.5 h. A solution of cyclosporin A (1.0 g, 0.83 mmol) in THF (8 mL) was added, and then the mixture was stirred for 2 h at −78° C. Paraformaldehyde (8.0 g) was heated to 170° C. and the resulting formaldehyde gas was transferred into the reaction via a glass tube which was wrapped with cotton and aluminum foil over 2 h. After stirred another 1 h at −78° C., the reaction mixture was quenched with water (10 mL). The mixture was allowed to warm to room temperature, diluted with ethyl acetate (150 mL) and washed with water (2×50 mL). The organic layer was separated, dried over anhydrous sodium sulfate and concentrated under vacuum. The crude material was purified by semi-preparative HPLC to afford cyclosporin diol (0.45 g, 44%) as a white solid: ¹H NMR (300 MHz, CDCl₃) δ 8.09 (d, J=9.9 Hz, 1H), 7.70 (d, J=7.4 Hz, 1H), 7.57 (d, J=8.2 Hz, 1H), 7.15 (overlapped with CHCl₃, 1H), 5.70 (dd, J=11.0, 4.0 Hz, 1H), 5.49 (d, J=6.4 Hz, 1H), 5.38-5.30 (m, 3H), 5.16-4.93 (m, 5H), 4.83 (t, J=7.2 Hz, 1H), 4.65 (t, J=9.5 Hz, 1H), 4.54 (t, J=7.2 Hz, 1H), 4.05 (d, J=6.8 Hz, 2H), 3.73 (t, J=6.3 Hz, 1H), 3.49 (s, 3H), 3.30 (s, 3H), 3.25 (s, 3H), 3.15 (s, 3H), 3.11 (s, 3H), 2.70 (s, 3H), 2.69 (s, 3H), 2.50-2.38 (m, 2H), 2.20-1.92 (m, 6H), 1.75-0.65 (m, 64H); ESI MS m/z 1233 [C₆₃H₁₁₃N₁₁O₁₃+H]⁺.

### Example 64

### Preparation of Cyclosporin Diacetate

To a solution of cyclosporin diol from Example 63 (0.43 g, 0.35 mmol) in methylene chloride (5 mL) was added pyridine (0.57 mL, 7.0 mmol) followed by 4-(dimethylamino)pyridine (86 mg, 0.70 mmol) and acetic anhydride (1.0 mL, 10.5 mmol). The reaction mixture was stirred for 2 days at room temperature. The reaction was diluted with ethyl ether (150 mL), washed with a saturated solution of sodium bicarbonate (30 mL), 1N HCl solution (30 mL) and brine (30 mL). The organic layer was separated, dried over anhydrous sodium sulfate and concentrated under vacuum. The crude material was purified by semi-preparative HPLC to afford cyclosporin diacetate (0.23 g, 50%) as a white solid: ¹H NMR (300 MHz, CDCl₃) δ 8.60 (d, J=9.8 Hz, 1H), 8.05 (d, J=6.6 Hz, 1H), 7.55 (d, J=7.8 Hz, 1H), 7.49 (d, J=9.3 Hz, 1H), 5.68 (dd, J=11.0, 4.0 Hz, 1H), 5.49 (s, 2H), 5.40-4.95 (m, 8H), 4.85 (t, J=7.5 Hz, 1H), 4.76 (t, J=9.3 Hz, 1H), 4.58-4.34 (m, 3H), 3.37 (s, 3H), 3.27 (s, 3H), 3.23 (s, 3H), 3.20 (s, 3H), 3.14 (s, 3H), 2.67 (s, 3H), 2.66 (s, 3H), 2.48-2.35 (m, 1H), 2.10 (s, 3H), 2.01 (s, 3H), 1.98-1.85 (m, 2H), 1.75-0.65 (m, 67H); ESI MS m/z 1317 [C₆₇H₁₁₇N₁₁O₁₅+H]⁺.

### Example 65

### Preparation of Cyclosporin Aldehyde

Ozone was bubbled into a solution of cyclosporin diacetate from Example 64 (0.22 g, 0.17 mmol) in methylene chloride (10 mL) at −78° C. until a blue color was developed. The mixture was degassed with nitrogen for a few min and dimethylsulfide (0.4 mL) was added at −78° C. The reaction mixture was allowed to warm to room temperature and stirred for 3 h. The reaction mixture was concentrated in vacuo and the residue was dissolved in ethyl acetate (120 mL), washed with water (2×20 mL) and brine (30 mL), dried over sodium sulfate, filtered, and concentrated in vacuo to afford cyclosporin aldehyde (0.19 g, 86%) as a white solid. The crude was carried to the next step without further purification: ¹H NMR (300 MHz, CDCl₃) δ 9.55 (d, J=3.4 Hz, 1H), 8.60 (d, J=9.9 Hz, 1H), 7.96 (d, J=7.1 Hz, 1H), 7.53 (d, J=7.7 Hz, 1H), 7.33 (d, J=9.1 Hz, 1H), 5.68 (dd, J=11.0, 4.0 Hz, 1H), 5.53 (d, J=11.2 Hz, 1H), 5.47 (d, J=11.2 Hz, 1H), 5.30 (dd, J=12.3, 3.6 Hz, 1H), 5.18-4.92 (m, 5H), 4.84 (t, J=6.9 Hz, 1H), 4.72 (t, J=9.6 Hz, 1H), 4.55-4.35 (m, 3H), 3.39 (s, 3H), 3.30 (s, 3H), 3.29 (s, 3H), 3.21 (s, 3H), 3.12 (s, 3H), 2.66 (s, 3H), 2.65 (s, 3H), 2.48-2.30 (m, 3H), 2.10 (s, 3H), 1.99 (s, 3H), 1.80-0.75 (m, 64H); ESI MS m/z 1305 [C₆₅H₁₁₃N₁₁O₁₆+H]⁺.

### Example 66

### Preparation of the Diacetate of trans-Cyclosporin Diene

To a suspension of bis(cyclopentadienyl)zirconiumchloride hydride (199 mg, 0.77 mmol) in methylene chloride (2 mL) was added propargyltrimethylsilane (0.12 mL, 0.81 mmol), and then the mixture was stirred at room temperature for 10 min. To this solution was sequentially added a solution of cyclosporin aldehyde from Example 65 (100 mg, 0.077 mmol) in methylene chloride (1 mL) and then silver perchlorate (3 mg, 0.015 mmol). The resulting mixture was stirred at room temperature for 12 h, and then poured into a saturated solution of sodium bicarbonate (10 mL). The organic layer was separated and the aqueous layer was extracted with methylene chloride (2×20 mL). The combined organics were dried over anhydrous sodium sulfate and concentrated under vacuum to afford the crude product. The material was purified by semi-preparative HPLC to afford the diacetate of trans-cyclosporin diene (47 mg, 46%) as a white solid: ¹H NMR (300 MHz, CDCl₃) δ 8.61 (d, J=9.5 Hz, 1H), 8.06 (d, J=6.8 Hz, 1H), 7.62 (d, J=9.2 Hz, 1H), 7.49 (d, J=7.7 Hz, 1H), 6.22 (dt, J=16.9, 10.2 Hz, 1H), 5.88 (dd, J=15.0, 10.5 Hz, 1H), 5.68 (dd, J=11.0, 4.0 Hz, 1H), 5.50 (s, 2H), 5.40-4.95 (m, 8H), 4.85 (t, J=7.5 Hz, 1H), 4.77 (t, J=9.3 Hz, 1H), 4.58-4.34 (m, 3H), 3.37 (s, 3H), 3.26 (s, 3H), 3.21 (s, 3H), 3.19 (s, 3H), 3.14 (s, 3H), 2.68 (s, 6H), 2.48-2.35 (m, 1H), 2.10 (s, 3H), 2.02 (s, 3H), 1.80-1.65 (m, 5H), 1.50-0.80 (m, 62H); ESI MS m/z 1329 [C₆₈H₁₁₇N₁₁O₁₅+H]⁺.

### Example 67

### Preparation of trans-Cyclosporin Diene

To a stirred solution of the diacetate of trans-cyclosporin diene from Example 66 (45 mg, 0.034 mmol) in methanol (2 mL) was added potassium carbonate (140 mg, 1.02 mmol) at room temperature. After 12 h at room temperature, the reaction mixture was diluted with ethyl acetate (100 mL) and washed with water (20 mL). The aqueous layer was separated and extracted with ethyl acetate (30 mL). The combined organics were dried over anhydrous sodium sulfate, and concentrated under vacuum to afford the crude product. The material was purified by semi-preparative HPLC to afford trans-cyclosporin diene (11 mg, 26%) as a white solid:

¹H NMR (300 MHz, CDCl₃) δ 8.07 (d, J=9.8 Hz, 1H), 7.66 (d, J=7.5 Hz, 1H), 7.52 (d, J=8.2 Hz, 1H), 7.18 (d, J=7.8 Hz, 1H), 6.29 (dt, J=16.9, 10.3 Hz, 1H), 5.98 (dd, J=15.0, 10.5 Hz, 1H), 5.69 (dd, J=11.0, 4.0 Hz, 1H), 5.65-5.55 (m, 1H), 5.51 (d, J=6.2 Hz, 1H), 5.30 (dd, J=11.6, 3.7 Hz, 1H), 5.15-4.93 (m 7H), 4.82 (t, J=7.5 Hz, 1H), 4.64 (t, J=9.4 Hz, 1H), 4.54 (t, J=7.4 Hz, 1H), 4.04 (d, J=6.7 Hz, 2H), 3.74 (t, J=6.9 Hz, 1H), 3.51 (s, 3H), 3.30 (s, 3H), 3.26 (s, 3H), 345 (s, 3H), 3.11 (s, 3H), 2.70 (s, 3H), 2.69 (s, 3H), 2.55-2.38 (m, 2H), 2.20-1.95 (m, 5H), 1.80-1.60 (m, 5H), 1.50-0.70 (m, 57H); ESI MS m/z 1245 [C₆₄H₁₁₃N₁₁O₁₃+H]⁺; HPLC>99% (AUC), t_{R}=14.05 min.

### Example 68

### Preparation of the Acetate of trans-Cyclosporin Diene-d₂

To a suspension of bis(cyclopentadienyl)zirconiumchloride hydride (199 mg, 0.77 mmol) in methylene chloride (2 mL) was added d₂-propargyltrimethylsilane (92 mg, 0.81 mmol), and then the mixture was stirred at room temperature for 10 min. To this solution was sequentially added a solution of cyclosporin aldehyde from Example 65 (100 mg, 0.077 mmol) in methylene chloride (1 mL) and then silver perchlorate (3 mg, 0.015 mmol). The resulting mixture was stirred at room temperature for 12 h, and then poured into a saturated solution of sodium bicarbonate (10 mL). The organic layer was separated and the aqueous layer was extracted with methylene chloride (2×20 mL). The combined organics were dried over anhydrous sodium sulfate and concentrated under vacuum to afford the crude product. The material was purified by semi-preparative HPLC to afford the acetate of deuterated trans-cyclosporin diene (20 mg, 20%) as a white solid: ¹H NMR (300 MHz, CDCl₃) δ 8.61 (d, J=9.5 Hz, 1H), 8.05 (d, J=6.8 Hz, 1H), 7.65-7.58 (m, 2H), 6.20 (d, J=10.5 Hz, 1H), 5.88 (dd, J=15.0, 10.5 Hz, 1H), 5.69 (d, J=7.5 Hz, 1H), 5.50 (s, 2H), 5.40-4.70 (m, 10H), 4.55-4.35 (m, 4H), 3.37 (s, 3H), 3.26 (s, 3H), 3.21 (s, 3H), 3.19 (s, 3H), 3.14 (s, 3H), 2.68 (s, 6H), 2.48-2.35 (m, 1H), 2.10 (s, 3H), 2.02 (s, 3H), 1.80-1.65 (m, 5H), 1.50-0.80 (m, 59H); ESI MS m/z 1331 [C₆₈H₁₁₅D₂N₁₁O₁₅+H]⁺.

### Example 69

### Preparation of trans-Cyclosporin Diene-d₂

To a stirred solution of the acetate of deuterated trans-cyclosporin diene from Example 68 (17 mg, 0.013 mmol) in methanol (3 mL) was added potassium carbonate (54 mg, 0.39 mmol) at room temperature. After 12 h at room temperature, the reaction mixture was diluted with ethyl acetate (100 mL) and washed with water (20 mL). The aqueous layer was separated and extracted with ethyl acetate (30 mL). The combined organics were dried over anhydrous sodium sulfate, and concentrated under vacuum to afford crude product. The material was purified by semi-preparative HPLC to afford trans-cyclosporin diene-d₂ (5 mg, 31%) as a white solid: ¹H NMR (300 MHz, CDCl₃) δ 8.06 (d, J=9.7 Hz, 1H), 7.66 (d, J=7.4 Hz, 1H), 7.48 (d, J=8.3 Hz, 1H), 7.16 (d, J=7.9 Hz, 1H), 6.29 (d, J=10.3 Hz, 1H), 5.98 (dd, J=15.0, 10.5 Hz, 1H), 5.69 (dd, J=11.0, 4.0 Hz, 1H), 5.65-5.55 (m, 1H), 5.51 (d, J=6.2 Hz, 1H), 5.30 (dd, J=11.6, 3.7 Hz, 1H), 5.15-4.75 (m, 8H), 4.65 (t, J=9.4 Hz, 1H), 4.53 (t, J=7.4 Hz, 1H), 4.04 (d, J=6.7 Hz, 2H), 3.75 (t, J=6.9 Hz, 1H), 3.51 (s, 3H), 3.31 (s, 3H), 3.26 (s, 3H), 3.15 (s, 3H), 3.11 (s, 3H), 2.70 (s, 3H), 2.68 (s, 3H), 2.55-2.30 (m, 2H), 2.20-1.60 (m, 10H), 1.50-0.70 (m, 55H); ESI MS m/z 1247 [C₆₄H₁₁₁D₂N₁₁O₁₃+H]⁺; HPLC 96.7% (AUC), t_{R}=13.76 min.

### Example 70

### Preparation of the Acetate of Cyclosporin Vinyl Chloride

Anhydrous CrCl₂ (119 mg, 0.97 mmol) was added to a solution of cyclosporin aldehyde from Example 65 (126 mg, 0.097 mmol) and CHCl₃ (30 mg, 0.25 mmol) in THF (4 mL) under argon atmosphere. The mixture was stirred at 40° C. under argon for 24 h, and then cooled down to room temperature, and filtered through a short silica gel column (EtOAc). The combined filtration washed with water (3×10 mL) and brine (3×10 mL), dried over Na₂SO₄, and concentrated to dryness. The residue was purified by semi-preparative HPLC to give the acetate of cyclosporin vinyl chloride (71 mg, 55%) as a white solid: ¹H NMR (CDCl₃, 300 MHz) δ 8.57 (d, J=9.7 Hz, 1H), 8.00 (d, J=6.6 Hz, 1H), 7.71 (d, J=7.5 Hz, 1H), 7.63 (d, J=9.0 Hz, 1H), 5.82-5.71 (m, 2H), 5.68 (dd, J=11.0, 4.2 Hz, 1H), 5.48 (d, J=6.8 Hz, 1H), 5.43 (dd, J=11.7, 3.8 Hz, 1H), 5.27 (dd, J=12.0, 3.9 Hz, 1H), 5.17 (t, J=7.5 Hz, 1H), 5.08-4.95 (m, 5H), 4.85 (t, J=7.2 Hz, 1H), 4.78 (t, J=9.6 Hz, 1H), 4.55-4.35 (m, 3H), 3.25 (s, 3H), 3.27 (s, 3H), 3.24 (s, 3H), 3.19 (s, 3H), 3.15 (s, 3H), 2.68 (s, 6H), 2.41-2.37 (m, 2H), 2.14-1.99 (m, 12H), 1.72-0.75 (m, 58H); ESI MS m/z 1337 [C₆₆H₁₁₄ClN₁₁O₁₅+H]⁺.

### Example 71

### Preparation of Cyclosporin Vinyl Chloride

The acetate of cyclosporin vinyl chloride from Example 70 (71 mg, 0.053 mmol) was dissolved in MeOH (7 mL), and then K₂CO₃ (200 mg, 1.449 mmol) was added. The mixture was stirred at room temperature under N₂ for 6 h, and then diluted with EtOAc (200 mL), washed with brine (3×10 mL), dried over Na₂SO₄, and concentrated to dryness. The residue was purified by semi-preparative HPLC to give cyclosporin vinyl chloride (35 mg, 53%) as a white solid: ¹H NMR (CDCl₃, 500 MHz) δ 8.08 (d, J=9.9 Hz, 1H), 7.69 (d, J=7.3 Hz, 1H), 7.50 (d, J=8.3 Hz, 1H), 7.26 (d, J=8.9 Hz, 1H), 5.92-5.84 (m, 2H), 5.70 (dd, J=11.0, 4.2 Hz, 1H), 5.48 (d, J=6.8 Hz, 1H), 5.33 (dd, J=11.7, 3.8 Hz, 1H), 5.10-4.95 (m, 5H), 4.83 (t, J=7.3 Hz, 1H), 4.65 (t, J=9.0 Hz, 1H), 4.50 (t, J=7.3 Hz, 1H), 4.04 (d, J=6.7 Hz, 2H), 3.80 (t, J=6.5 Hz, 1H), 3.50 (s, 3H), 3.31 (s, 3H), 3.27 (s, 3H), 3.15 (s, 3H), 3.12 (s, 3H), 2.69 (s, 3H), 2.68 (s, 3H), 2.41-2.37 (m, 2H), 2.14-1.99 (m, 6H), 1.78-1.64 (m, 7H), 1.34-0.80 (m, 54H); ESI MS m/z 1253 [C₆₂H₁₁₀ClN₁₁O₁₃+H]⁺; HPLC>99% (AUC), t_{R}=14.0 min.

### Example 72

### Preparation of Cyclosporin Vinyl Iodide

To an ice-cooled solution of cyclosporin aldehyde from Example 65 (200 mg, 0.15 mmol) in THF (5 mL) was added anhydrous CrCl₂ (184 mg, 1.5 mmol) and CHI₃ (206 mg, 0.6 mmol), and then the mixture was stirred at 0° C. under N₂ for 24 h. The reaction mixture was diluted with EtOAc (200 mL), washed with water (4×10 mL), dried over mgSO₄, and concentrated to dryness. The residue was purified by semi-preparative HPLC to give the diacetate of cyclosporin vinyl iodide (57 mg, 26%) as a pale yellow oil: ESI MS m/z 1429 [C₆₆H₁₁₄IN₁₁O₁₅+H]⁺.

The above diacetate of cyclosporine vinyl iodide (57 mg, 0.04 mmol) was dissolved in MeOH (10 mL), and then K₂CO₃ (200 mg, 1.45 mmol) was added. The mixture was stirred at room temperature under N₂ overnight, and then diluted with EtOAc (200 mL), washed with brine (3×10 mL), dried over Na₂SO₄, and concentrated to dryness. The residue was purified by semi-preparative HPLC to give cyclosporin vinyl iodide (19 mg, 35%) as a white solid: ¹H NMR (CDCl₃,500 MHz) δ 8.05 (d, J=9.8 Hz, 1H), 7.68 (d, J=7.4 Hz, 1H), 7.48 (d, J=9.1 Hz, 1H), 7.22 (d, J=7.8 Hz, 1H), 6.49 (ddd, J=14.5, 8.5, 6.3 Hz, 1H), 5.91 (d, J=14.3 Hz, 1H), 5.70 (dd, J=10.9, 4.0 Hz, 1H), 5.49 (d, J=6.6 Hz, 1H), 5.31 (dd, J=12.7, 3.8 Hz, 1H), 5.09-5.04 (m, 4H), 4.98-4.94 (m, 2H), 4.84 (t, J=7.2 Hz, 1H), 4.65 (dd, J=9.6, 8.5 Hz, 1H), 4.53 (t, J=7.3 Hz, 1H), 4.04 (d, J=6.7 Hz, 1H), 3.76 (t, J=6.5 Hz, 1H), 3.50 (s, 3H), 3.30 (s, 3H), 3.27 (s, 3H), 3.15 (s, 3H), 3.11 (s, 3H), 2.69 (s, 3H), 2.68 (s, 3H), 2.45-2.40 (m, 2H), 1.78-1.64 (m, 7H), 1.43-0.76 (m, 60H); ESI MS m/z 1344 [C₆₂H₁₁₀IN₁₁O₁₃+H]⁺; HPLC 95.6% (AUC), t_{R}=14.1 min.

### Example 73

### Preparation of the Acetates of cis- and trans-Cyclosporin Vinyl Bromide

To a suspension of (bromomethyl)triphenylphosphonium bromide (700 mg, 1.6 mmol) in THF (5 mL) at −78° C. was added dropwise sodium bis(trimethylsilyl)amide (1.6 mL, 1 M in THF, 1.6 mmol), then the mixture was stirred for 1 h. A solution of cyclosporin aldehyde from Example 65 (0.21 g, 0.16 mmol) in THF (5 mL) was added, and then the mixture was stirred for 2 h at −78° C. The reaction mixture was quenched with a saturated solution of ammonium chloride. After warmed to room temperature, the mixture was diluted with ethyl ether (100 mL), washed with brine (30 mL), dried over anhydrous sodium sulfate and concentrated under vacuum. The crude material was purified by semi-preparative HPLC to afford the acetate of cis-cyclosporin vinyl bromide (50 mg, 23%): ¹H NMR (CDCl₃, 300 MHz) δ 8.62 (d, J=9.7 Hz, 1H), 8.01 (d, J=6.6 Hz, 1H), 7.60 (d, J=7.8 Hz, 1H), 7.54 (d, J=9.0 Hz, 1H), 6.04 (d, J=7.7 Hz, 1H), 6.02-5.92 (m, 1H), 5.69 (dd, J=10.8, 3.9 Hz, 1H), 5.50 (dd, J=16.5, 11.5 Hz, 2H), 5.24 (dd, J=12.2, 3.7 Hz, 2H), 5.15 (dd, J=7.5, 6.0 Hz, 1H), 5.10-4.93 (m, 3H), 4.95 (t, J=7.5 Hz, 1H), 4.73 (t, J=9.6 Hz, 1H), 4.55-4.38 (m, 4H), 3.37 (s, 3H), 3.29 (s, 3H), 3.23 (s, 3H), 3.19 (s, 3H), 3.14 (s, 3H), 2.67 (s, 3H), 2.66 (s, 3H), 2.45-2.35 (m, 1H), 2.20-2.15 (m, 2H), 2.10 (s, 3H), 2.04 (s, 3H), 2.00-1.82 (m, 3H), 1.80-1.60 (m, 3H), 1.48-0.82 (m, 57H); ESI MS m/z 1381 [C₆₆H₁₁₄BrN₁₁O₁₅+H]⁺; and the acetate of trans-cyclosporin vinyl bromide (8 mg, 4%): ¹H NMR (CDCl₃, 300 MHz) δ 8.54 (d, J=9.9 Hz, 1H), 7.99 (d, J=6.9 Hz, 1H), 7.56 (d, J=7.8 Hz, 1H), 7.38 (d, J=9.2 Hz, 1H), 6.15-5.92 (m, 1H), 5.85 (d, J=13.8 Hz, 1H), 5.72-5.65 (m, 1H), 5.54-5.43 (m, 2H), 5.38 (dd, J=11.7, 3.9 Hz, 1H), 5.78-5.69 (m, 1H), 5.15 (t, J=5.7 Hz, 1H), 5.04-4.70 (m, 5H), 4.54-4.30 (m, 2H), 4.04 (t, J=4.0 Hz, 1H), 3.35 (s, 3H), 3.28 (s, 3H), 3.26 (s, 3H), 3.20 (s, 3H), 3.14 (s, 3H), 2.67 (s, 3H), 2.66 (s, 3H), 2.42-2.33 (m, 1H), 2.25-2.12 (m, 2H), 2.10 (s, 3H), 2.02 (s, 3H), 1.90-1.62 (m, 6H), 1.45-0.85 (m, 58H); ESI MS m/z 1381 [C₆₆H₁₁₄BrN₁₁O₁₅+H]⁺.

### Example 74

### Preparation of cis-Cyclosporin Vinyl Bromide

To a stirred solution of the acetate of cis-cyclosporin vinyl bromide from Example 73 (24 mg, 0.017 mmol) in methanol (3 mL) was added potassium carbonate (120 mg, 0.86 mmol) at room temperature. After 12 h at room temperature, the reaction mixture was quenched with a saturated solution of ammonium chloride (20 mL), and then extracted with ethyl acetate (3×30 mL). The combined organics were dried over anhydrous sodium sulfate and concentrated under vacuum to afford the crude product. The material was purified by semi-preparative HPLC to afford cis-cyclosporin vinyl bromide (8 mg, 36%) as a white solid: ¹H NMR (300 MHz, CDCl₃) δ 8.15 (d, J=9.9 Hz, 1H), 7.74 (d, J=7.2 Hz, 1H), 7.62 (d, J=8.1 Hz, 1H), 7.39 (d, J=8.0 Hz, 1H), 6.14-6.02 (m, 2H), 5.69 (dd, J=11.1, 3.9 Hz, 1H), 5.43 (d, J=7.2 Hz, 1H), 5.30 (dd, J=11.4, 3.6 Hz, 1H), 5.15-5.38 (m, 6H), 4.83 (t, J=6.9 Hz, 1H), 4.66 (t, J=9.0 Hz, 1H), 4.52 (t, J=7.2 Hz, 1H), 4.05 (d, J=6.6 Hz, 2H), 3.90 (t, J=6.6 Hz, 1H), 3.50 (s, 3H), 3.31 (s, 3H), 3.25 (s, 3H), 3.15 (s, 3H), 3.12 (s, 3H), 2.70 (s, 3H), 2.69 (s, 3H), 2.43-1.90 (m, 8H), 1.80-1.57 (m, 5H), 1.45-0.75 (m, 55H); ESI MS m/z 1297 [C₆₂H₁₁BrN₁₁O₁₃+H]⁺; HPLC 97.0% (AUC), t_{R}=13.95 min.

### Example 75

### Preparation of trans-Cyclosporin Vinyl Bromide

To a stirred solution of the acetate of trans-cyclosporin vinyl bromide from Example 73 (10 mg, 0.007 mmol) in methanol (2 mL) was added potassium carbonate (50 mg, 0.36 mmol) at room temperature. After 12 h at room temperature, the reaction mixture was quenched with a saturated solution of ammonium chloride (15 mL), and then extracted with ethyl acetate (3×30 mL). The combined organics were dried over anhydrous sodium sulfate and concentrated under vacuum to afford the crude product. The material was purified by semi-preparative HPLC to afford trans-cyclosporin vinyl bromide (4 mg, 44%) as a white solid: ¹H NMR (300 MHz, CDCl₃) δ 8.08 (d, J=9.9 Hz, 1H), 7.71 (d, J=7.2 Hz, 1H), 7.56 (d, J=8.1 Hz, 1H), 7.31 (d, J=8.0 Hz, 1H), 6.20-6.05 (m, 1H), 5.94 (d, J=13.4 Hz, 1H), 5.70 (dd, J=10.8, 3.6 Hz, 1H), 5.49 (d, J=6.6 Hz, 1H), 5.34 (dd, J=11.4, 3.6 Hz, 1H), 5.12-4.95 (m, 6H), 4.83 (t, J=6.9 Hz, 1H), 4.66 (t, J=9.0 Hz, 1H), 4.51 (t, J=7.2 Hz, 1H), 4.05 (d, J=6.6 Hz, 2H), 3.78 (t, J=6.0 Hz, 1H), 3.50 (s, 3H), 3.30 (s, 3H), 3.26 (s, 3H), 3.15 (s, 3H), 3.11 (s, 3H), 2.70 (s, 3H), 2.69 (s, 3H), 2.42-2.33 (m, 2H), 2.20-1.89 (m, 6H), 1.80-1.60 (m, 5H), 1.45-0.75 (m, 55H); ESI MS m/z 1297 [C₆₂H₁₁₀BrN₁₁O₁₃+H]⁺; HPLC 97.0% (AUC), t_{R}=13.74 min.

### Example 76

### Preparation of Arylated Cyclosporin Diol

To a stirred solution of cyclosporin diol from Example 63 (57 mg, 0.040 mmol) in methylene chloride was added styrene (42 mg, 0.400 mmol) and Grubbs' catalyst 2^{nd} generation (2.5 mg, 0.004 mmol). The resulting mixture was stirred overnight while refluxing at 50° C. in a nitrogen atmosphere. The reaction was then cooled to 25° C. and concentrated to dryness. The crude mixture was purified by semi-preparative HPLC twice to afford the desired product (8.8 mg, 17%) as a white solid: ¹H NMR (300 MHz, CDCl₃); δ 8.09 (d, J=9.9 Hz, 1H), 7.70 (d, J=7.7 Hz, 1H), 7.60-7.08 (m, 6H), 6.98-6.90 (m, 1H), 6.33 (d, J=15.7 Hz, 1H), 6.20-6.12 (m, 1H), 5.69 (dd, J=10.8, 4.0 Hz, 1H), 5.58 (d, J=5.6 Hz, 1H), 5.32 (dd, J=11.6, 3.6 Hz, 1H), 5.1144.91 (m, 5H), 4.82 (t, J=7.1 Hz, 1H), 4.68-4.50 (m, 2H), 4.04 (d, J=6.8 Hz, 2H), 3.72 (t, J=6.0 Hz, 1H), 3.53 (s, 3H), 3.31 (s, 3H), 3.27 (s, 3H), 3.16 (s, 3H), 3.10 (s, 3H), 2.71 (s, 3H), 2.68 (s, 3H), 2.45-2.35 (m, 1H), 2.15-1.90 (m, 5H), 1.78-1.52 (m, 7H), 1.48-0.65 (m, 56H); ESI MS m/z 1295 [C₆₈H₁₁₅N₁₁O₁₃+H]⁺; HPLC 93.8% (AUC), t_{R}=14.26 min.

### Example 77

### Preparation of Cyclosporin Fluoride

A flask charged with a solution of cyclosporin diol from Example 63 (50 mg, 0.410 mmol) in methylene chloride (2 mL) was cooled to −78° C. Allyl Fluoride (1.5 g, 90.11 mmol) was bubbled through the solution. The reaction was allowed to warm to room temperature and Grubbs' catalyst 2^{nd} generation (18 mg, 0.021 mmol) was added. The resulting mixture was stirred overnight while refluxing at 50° C. in an atmosphere of allyl fluoride (via attached balloon). After 16 h, the reaction was concentrated to dryness under reduced pressure. Purification by semi-preparative HPLC yielded 11.3 mg (22%) of the cyclosporin fluoride as an off-white solid: ¹H NMR (500 MHz, CDCl₃) 8.11 (d, J=9.5 Hz, 1H), 7.69 (d, J=7.5 Hz, 1H), 7.59 (d, J=8.0 Hz, 1H), 7.25 (overlapped with CHCl₃, 1H), 5.79-5.57 (m, 3H), 5.51 (d, J=6.5 Hz, 1H), 5.29 (dd, J=12.0, 4.0 Hz, 1H), 5.12-4.94 (m, 5H), 4.87-4.79 (m, 2H), 4.72 (d, J=6.0 Hz, 1H), 4.64 (t, J=8.5 Hz, 1H), 4.55 (t, J=7.5 Hz, 1H), 4.04 (d, J=6.5 Hz, 2H), 3.75 (t, J=7.0 Hz, 1H), 3.50 (s, 3H), 3.23 (s, 3H), 3.26 (s, 3H), 3.15 (s, 3H), 3.11 (s, 3H), 2.70 (s, 3H), 2.69 (s, 3H), 2.53-2.37 (m, 2H), 2.18-1.9(m, 6H), 1.82-1.60 (m, 7H), 1.52-0.70 (m, 54H); ESI MS m/z 1291 [C₆₃H₁₁₂FN₁₁O₁₃+H]⁺; HPLC 98.3% (AUC), t_{R}=13.42 min.

### Example 78

### Preparation of Cyclosporin Trifluoride

To a dried 25 mL flask charged with a solution of cyclosporin diol from Example 63 (50 mg, 0.041 mmol) in methylene chloride (2 mL) was added 3,3,3-trifluoropropene gas (39 mg, 0.41 mmol). The solution was treated with Grubbs' catalyst 2^{nd} generation (18 mg, 0.021 mmol) and the resulting mixture was allowed to stir while refluxing overnight at 50° C. in an atmosphere of 3,3,3-trifluoropropene gas (via attached balloon). After 17 h, Grubbs' catalyst 2^{nd} generation (18 mg, 0.021 mmol) was added and the reaction was left to reflux overnight at 50° C. in an atmosphere of 3,3,3-trifluoropropene gas. The reaction was concentrated to dryness under reduced pressure. Purification by semi-preparative HPLC yielded the desired product (2 mg, 4%) as a pink solid: ¹H NMR (300 MHz, CDCl₃) δ 8.06 (d, J=10.0 Hz, 1H), 7.68 (d, J=8.0 Hz, 1H), 7.52 (d, J=8.5 Hz, 1H), 7.20 (d, J=8.5 Hz, 1H), 6.42-6.33 (m, 1H), 5.70 (dd, J=11.0, 8.5 Hz, 1H), 5.62-5.53 (m, 2H), 5.26 (dd, J=12.0, 4.0 Hz, 1H), 5.11-5.03 (m, 4H), 5.01-4.92 (m, 2H), 4.83 (t, J=7.0 Hz, 1H), 4.63 (t, J=9.5 Hz, 1H), 4.55 (t, J=7.0 Hz, 1H), 4.04 (d, J=6.0 Hz, 2H), 3.76 (t, J=7.0 Hz, 1H), 3.52 (s, 3H), 3.30 (s, 3H), 3.27 (s, 3H), 3.16 (s, 3H), 3.11 (s, 3H), 2.70 (s, 3H), 2.68 (s, 3H), 2.63-2.52 (m, 1H), 2.47-2.31 (m, 2H), 2.19-0.71 (m, 65H); ESI MS m/z 1287 [C₆₃H₁₁₀F₃N₁₁O₁₃+H]⁺; HPLC 94.3% (AUC), t_{R}=13.17 min.

### Example 79

### Preparation of the Acetate of Cyclosporin α,β-Unsaturated Aldehyde

A mixture of cyclosporin diacetate from Example 64 (100 mg, 0.076 mmol), acrolein dimethyl acetal (0.087 mL, 0.76 mmol), Grubbs' catalyst 2^{nd} generation (12.7 mg, 0.015 mmol) and toluene (2 mL) was heated at 55° C. in a 25 mL flask overnight. The catalyst (12.7 mg) and acrolein dimethyl acetal (0.087 mL) were refilled and the mixture was stirred at the same temperature for an additional 24 h. The catalyst (12.7 mg) was again refilled and the reaction was allowed to stir at 55° C. for 6 h. An additional 20 mg of catalyst was added and the reaction was allowed to stir at 55° C. overnight. The catalyst (32.3 mg) was refilled again and an additional 1 mL of acrolein dimethyl acetal was added. After 2 days at 55° C., 20 mg of Grubbs' catalyst was added as well as 0.017 mL of acrolein dimethyl acetal. After 24 h, the reaction was cooled to room temperature and concentrated in vacuo. The residue was purified by semi-preparative HPLC to afford the acetate of cyclosporin α,β-unsaturated aldehyde (40 mg, 40%): ¹H NMR (300 MHz, CDCl₃) δ 9.41 (d, J=7.9 Hz, 1H), 8.60 (d, J=9.7 Hz, 1H), 8.01 (d, J=6.8 Hz, 1H), 7.60 (d, J=8.0 Hz, 2H), 6.82-6.67 (m, 1H), 5.99 (dd, J=15.4, 7.8 Hz, 1H), 5.68 (dd, J=11.0, 3.9 Hz, 1H), 5.54 (s, 2H), 5.33-5.12 (m, 3H), 5.09-4.92 (m, 3H), 4.84 (t, J=7.2 Hz, 1H), 4.68 (t, J=9.4 Hz, 1H), 4.56-4.32 (m, 3H) 3.38 (s, 3H), 3.28 (s, 3H), 3.22 (s, 3H), 3.20 (s, 3H), 3.16 (s, 3H), 2.68 (s, 3H), 2.67 (s, 3H), 2.51-2.13 (m, 3H), 2.11 (s, 3H), 2.04 (s, 3H), 1.99-1.58 (m, 7H), 1.51-0.79 (m, 57H); ESI MS m/z 1331 [C₆₇H₁₁₅N₁₁O₁₆+H]⁺.

### Example 80

### Preparation of the Acetate of Cyclosporin Dienyl Chloride

To a dried 25 mL flask charged with a solution of the acetate of cyclosporin α,β-unsaturated aldehyde from Example 79 (71 mg, 0.053 mmol) and chloroform (63 mg, 0.53 mmol) in THF (3 mL) was added chromium chloride (195 mg, 1.59 mmol). The resulting mixture was heated to 50° C. and stirred under N₂ for 2 h. The reaction was then cooled to room temperature and poured into 200 mL of ice-water with vigorously stirring. The aqueous layer was then extracted with ethyl acetate (3×200 mL). The combined organics were washed with brine (80 mL) and dried over anhydrous sodium sulfate, and then concentrated under vacuum. The crude material was purified by semi-preparative HPLC to yield the acetate of cyclosporin dienyl chloride (44 mg, 63%) as a white solid: ¹H NMR (300 MHz, CDCl₃) δ 8.64 (d, J=9.9 Hz, 1H), 8.05 (d, J=13.3 Hz, 1H), 7.69 (d, J=9.0 Hz, 1H), 7.64 (d, J=7.8 Hz, 1H), 6.39 (dd, J=13.2, 10.7 Hz, 1H), 6.06 (d, J=13.3 Hz, 1H), 5.92-5.67 (m, 1H), 5.50 (s, 2H) 5.41-5.25 (m, 2H), 5.23-5.13 (m, 1H), 5.09-4.93 (m, 3H), 4.85 (t, J=7.1 Hz, 1H), 4.77 (t, J=9.4 Hz, 1H), 4.57-4.34 (m, 2H), 3.67 (s, 3H), 3.26 (s, 3H), 3.23 (s, 3H), 3.19 (s, 3H), 3.14 (s, 3H), 2.68 (s, 6H), 2.48-2.31 (m, 1H), 2.11 (s, 3H), 2.02 (s, 3H), 1.82-1.63 (m, 4H), 1.48-0.68 (m, 65H); ESI MS m/z 1364 [C₆₈H₁₁₆ClN₁₁O₁₅+H]⁺.

### Example 81

### Preparation of Cyclosporin Dienyl Chloride

A solution of the acetate of cyclosporin dienyl chloride from Example 80 (44 mg, 0.03 mmol) in MeOH (1 mL) was treated with potassium carbonate (83 mg, 0.60 mmol). This was allowed to stir at room temperature overnight. The reaction was then diluted with ethyl acetate (30 mL), washed with a saturated solution of sodium bicarbonate (20 mL), washed with brine (20 mL) and then dried over anhydrous sodium sulfate. It was then concentrated under reduced pressure. The crude material was purified by semi-preparative HPLC to yield cyclosporin dienyl chloride (6 mg, 16%): ¹H NMR (300 MHz, CDCl₃) δ 8.10 (d, J=9.9 Hz, 1H), 7.68 (d, J=7.5 Hz, 1H), 7.62 (d, J=8.8 Hz, 1H), 7.27 (d, J=6.4 Hz, 1H), 6.42 (dd, J=13.0, 10.8 Hz, 1H), 6.07 (d, J=13.1 Hz, 1H), 5.91 (dd, J=15.2, 10.9 Hz, 1H), 5.69 (dd, J=10.5, 3.6 Hz, 1H), 5.47-5.52 (m, 1H), 5.50 (d, J=6.3 Hz, 1H), 5.29 (dd, J=11.6, 3.9 Hz, 1H), 5.12-4.92 (m, 3H), 4.83 (t, J=7.3 Hz, 1H), 4.68-4.60 (m, 1H), 4.54 (t, J=7.2 Hz, 1H), 3.49 (s, 3H), 3.30 (s, 3H), 3.26 (s, 3H), 3.16 (s, 3H), 3.11 (s, 3H), 2.70 (s, 3H), 2.69 (s, 3H), 2.57-2.32 (m, 1H), 2.21-1.91 (m, 4H), 1.61-0.69 (m, 69H); ESI MS m/z 1280 [C₆₄H₁₂₂ClN₁₁O₁₃+H]⁺; HPLC 96.9% (AUC), t_{R}=13.98 min.

### Example 82

### Preparation of the Acetate of Cyclosporin Dienyl Iodide

A 25 mL flask charged with a solution of the acetate of cyclosporin α,β-unsaturated aldehyde from Example 79 (36 mg, 0.027 mmol) in THF (2 mL) was treated with iodoform (108 mg, 0.027 mmol). It was then cooled to −78° C. and chromium chloride (99 mg, 0.81 mmol) was added to the solution. The mixture was stirred at −78° C. for 10 min and then warmed to 0° C., at which it was stirred for 1.5 h. The reaction was then poured into 70 mL of vigorously stirring ice water. The material was extracted with 100 mL of ethyl acetate. The organic layer was rinsed with 15 mL of brine, dried over anhydrous sodium sulfate, and concentrated under reduced pressure. The crude material was purified by semi-preparative HPLC to yield the acetate of cyclosporin dienyl iodide (14 mg, 35%): ¹H NMR (300 MHz, CDCl₃) δ 8.64 (d, J=9.8 Hz, 1H), 8.05 (d, J=6.9 Hz, 1H), 7.71 (d, J=8.9 Hz, 1H), 7.66 (d, J=7.7 Hz, 1H), 6.95 (dd, J=14.2, 10.6 Hz, 1H), 6.15 (d, J=14.4 Hz, 1H), 5.81 (dd, J=14.8, 10.6 Hz, 1H), 5.69 (dd, J=10.8, 3.9 Hz, 1H), 5.50 (s, 2H), 5.43-5.27 (m, 3H), 5.22-5.13 (m, 1H), 5.09-4.95 (m, 3H), 4.85 (t, J=7.1 Hz, 1H), 4.78 (t, J=9.2 Hz, 1H), 4.58-4.32 (m, 3H), 3.37 (s, 3H), 3.25 (s, 3H), 3.19 (s, 3H), 3.14 (s, 3H), 2.68 (s, 6H), 2.49-2.32 (m, 1H), 2.11 (s, 3H), 2.02 (s, 3H), 1.97-1.57 (m, 4H), 1.48-0.71 (m, 65H); ESI MS m/z 1455 [C₆₈H₁₁₆IN₁₁O₁₅+H]⁺.

### Example 83

### Preparation of Cyclosporin Dienyl Iodide

A solution of the acetate of cyclosporin dienyl iodide from Example 82 (13.7 mg, 0.003 mmol) in MeOH (1 mL) was treated with potassium carbonate (26 mg, 0.19 mmol). This was allowed to stir at room temperature overnight. The reaction was then diluted with 30 mL of ethyl acetate, washed with a saturated solution of sodium bicarbonate (20 mL), washed with 20 mL of brine and then dried over anhydrous sodium sulfate. It was then concentrated under reduced pressure. The crude material was purified by semi-preparative HPLC to yield cyclosporin dienyl iodide (7.3 mg, 59%) as a white solid and a mixture of cis and trans-isomers:

¹H NMR (300 MHz, CDCl₃) δ 8.08 (d, J=10.1 Hz, 1H), 7.66 (d, J=7.3 Hz, 1H), 7.54-7.47 (m, 1H), 7.19 (d, J=7.8 Hz, 1H), 7.01 (dd, J=14.2, 10.8 Hz, 0.8H), 6.69 (dd, J=9.7, 7.4 Hz, 0.2H), 6.22-6.07 (m, 1H), 5.96-5.83 (m, 1H), 5.73-5.53 (m, 1H), 5.49 (t, J=6.9 Hz, 1H), 5.33-5.24 (m, 1H), 5.13-4.92 (m, 6H), 4.83 (t, J=7.3 Hz, 1H), 4.65 (t, J=8.6 Hz, 1H), 4.58-4.46 (m, 1H), 4.04 (d, J=6.7 Hz, 1H), 3.51 (s, 0.6H), 3.50 (s, 2.4H), 3.31(s, 3H), 3.27 (s, 2.4H), 3.26 (s, 0.6H), 3.15 (s, 3H), 3.12 (s, 3H), 2.69 (s, 1.2H), 2.68 (s, 4.8H) 2.52-2.43 (m, 1H), 2.32-0.72 (m, 70H); ESI MS m/z 1371 [C₆₄H₁₁₂IN₁₁O₁₃+H]⁺; HPLC 94.0% (AUC), t_{R}=14.22 min.

### Example 84

### Preparation of Cyclosporin Dienyl Bromide

A 25 mL flask charged with a solution of the acetate of cyclosporin α,β-unsaturated aldehyde from Example 79 (49 mg, 0.04 mmol) in THF (2 mL) was cooled to 0° C. and treated with bromoform (0.04 mL, 0.40 mmol). Chromium(II) chloride (147 mg, 1.20 mmol) was added to the solution. The mixture was stirred at 0° C. for 1.5 h and then the ice bath was removed. The mixture was left to stir at room temperature overnight. The reaction was then poured into 70 mL of vigorously stirring ice water. The material was extracted with 150 mL of ethyl acetate. The organic layer was rinsed with 25 mL of brine, dried over anhydrous sodium sulfate, and concentrated under reduced pressure. The crude material was purified by semi-preparative HPLC to yield the acetate of cyclosporin dienyl bromide (5.2 mg, 9%): ESI MS m/z 1408 [C₆₈H₁₁₅BrN₁₁O₁₅+H]⁺.

A solution of the above acetate of cyclosporin dienyl bromide (5.2 mg, 0.004 mmol) in MeOH (1 mL) was treated with potassium carbonate (21 mg, 0.15 mmol). This was allowed to stir at room temperature for 7 h. The reaction was then diluted with 60 mL of ethyl acetate, washed with a saturated solution of sodium bicarbonate (10 mL), washed with 10 mL of brine and then dried over anhydrous sodium sulfate. It was then concentrated under reduced pressure. The crude material was purified using semi-preparative HPLC to yield cyclosporin dienyl bromide (2.1 mg, 40%): ¹H NMR (300 MHz, CDCl₃) δ 8.10 (d, J=9.9 Hz, 1H), 7.73-7.62 (m, 1H), 7.58-7.58 (m, 1H), 7.18 (d, J=7.5 Hz, 1H), 6.70 (dd, J=13.4, 10.7 Hz, 1H), 6.16 (d, J=13.4 Hz, 1H), 5.88 (dd, J=15.0, 10.8 Hz, 1H), 5.73-5.57 (m, 1H), 5.48 (d, J=6.5 Hz, 1H), 5.38-5.23 (m, 2H), 5.12-4.92 (m, 6H), 4.83 (t, J=7.4 Hz, 1H), 4.65 (t, J=8.5 Hz, 1H), 4.52 (t, J=7.4 Hz, 1H), 4.22 (dd, J=5.7, 3.6 Hz, 1H), 4.04 (d, J=6.5 Hz, 1H), 3.50 (s, 3H), 3.31 (s, 3H), 3.27 (s, 3H), 3.15 (s, 3H), 3.12 (s, 3H), 2.69 (s, 3H), 2.68 (s, 3H), 2.49-2.26 (m, 1H), 2.22-0.67 (m, 68H); ESI MS m/z 1323 [C₆₄H₁₁₂BrN₁₁O₁₃+H]⁺; HPLC>99% (AUC), t_{R}=13.93 min.

### Example 85

### Concanavalin A-Stimulated Splenocyte Assay

Male BALB/c mice, at 5 to 7 weeks of age, were sacrificed by CO₂ inhalation. Spleens were removed and dissociated by pushing through a nylon cell strainer. The splenocytes were washed in RPMI 1640/5% fetal calf serum (FCS) and pelleted at 400×g. Red blood cells were then lysed by resuspending the cell pellet in ACK lysis buffer (150 mM NH₄Cl, 1 mM KHCO₃, 0.1 mM EDTA, 3 mL per spleen) for 10 min at room temperature. After pelleting at 400×g, the cells were washed by resuspending in RPMI 1640/5% FCS and repelleting. The cell pellet was resuspended in RPMI 1640/5% FCS and again passed through a cell strainer to remove cell aggregates. The cells were then counted and adjusted to 2×10⁶ cells/ml in RPMI 1640/10% FCS/50 μM 2-mercaptoethanol. Cell viability was assessed by Trypan blue staining. Cyclosporin A or the test compound and two micrograms of concanavalin A were added to the wells of a 96 well plate, prior to the addition of 2×10⁵ splenocytes. The cells were cultured in a 37° C. CO₂ incubator for 2 days and then pulsed with 1 μCi of [³H]thymidine for 6 hours. Cells were harvested onto filtermats with a TomTec 96 well plate harvester and lysed with H₂O. The filtermat and scintillation fluid were sealed in a plastic sleeve. [³H]thymidine incorporation was measured with a Wallac Trilux plate counter. Initial screens were done at a fixed value of 100 ng/ml test compound. IC₅₀s were calculated from 7 point concentration-response curves using GraphPad software.

### Example 86

### Murine Ex Vivo Pharmacodynamic Assay

In vivo immunosuppressive activity can be determined for cyclosporin

A and the disclosed cyclosporin analogs, as described below. The concanavalin A-stimulated splenocyte activity can be assessed in vivo using a method previously described by Peterson et al. (Peterson et al., “A Tacrolimus-Related Immunosuppressant with Biochemical Properties Distinct from Those of Tacrolimus,”Transplantation, 65:10-18 (1998), which is hereby incorporated by reference in its entirety) or a slightly modified version thereof.

Optimal doses of cyclosporin A or an immunosuppressive compound of the present invention (four different doses of test drug plus a control set of animals with no drug) was administered orally or intravenously to male BALB/c or female C57BL mice. Three mice were tested at each dose. Concanavalin A was injected into the tail vein of the mouse at 4 hours after the administration of cyclosporin A or the immunosuppressive compound. One hour after the concanavalin A injection, the mice were euthanized, the spleens were removed under sterile conditions, and the extent of splenocyte proliferation was measured in a similar manner as described in Example 85. The percent inhibition relative to control was plotted graphically versus the dose of the immunosuppressive compound and an ED₅₀ value was determined. Each dose-response assay for the compound of the present invention was accompanied by a cyclosporin control at a single dose equal to the ED₅₀.

### Example 87

### Assay for Inhibition of Peptidyl Prolyl Isomerase Activity of Cyclophilin A

The assay for inhibition of peptidyl prolyl isomerase activity of cyclophilin A is a modification of the procedure described by Kofron et al., “Determination of Kinetic Constants for Peptidyl Prolyl cis-trans Isomerases by an Improved Spectrophotometric Assay,” Biochemistry 30:6127-6134 (1991), which is hereby incorporated by reference in its entirety. Recombinant human cyclophilin A in 50 mM HEPES, 100 mM NaCl pH 8.0 is precooled to 4° C. Test compounds and the cyclosporin positive control are dissolved in dimethyl sulfoxide (DMSO) and introduced over a range of concentrations. Chymotrypsin is then added to a final concentration of 6 mg/ml. The peptide substrate, Suc-Ala-Ala-Pro-Phe-pNA, is dissolved in 470 mM LiCl in trifluoroethanol and then added to 25 μg/ml to initiate the reaction. After rapid mixing, the absorbance at 390 nm is monitored over a 90 second time course.

### Example 88

### Cellular Assay for Determination of HIV Inhibition

The in vitro anti-HIV activity of compounds of the present invention is measured in established cell line cultures as described by Mayaux et al., “Triterpene Derivatives That Block Entry of Human Immunodeficiency Virus Type 1 Into Cells,” Proc. Natl. Acad. Sci. USA 91:3564-3568 (1994), which is hereby incorporated by reference in its entirety. The CEM4 cell line was infected with HIV-1_{Lai} strain. The inhibition of HIV replication in the culture is estimated by the measure of the reverse transcriptase (RT) produced in the supernatant. Anti-viral activity is expressed as the IC₅₀ RT, the concentration required to reduce replication of HIV by 50%, and is determined by linear regression.

### Example 89

### Intracellular Replication of the HCV Genome In Vitro

The effect of the cyclosporin compounds of the present invention on the intracellular replication of the HCV genome in vitro, using an HCV replicon system in a cultured human hepatoma Huh7 cell line is determined by the method of Lohmann et al., “Replication of Subgenomic Hepatitis C Virus RNAs in a Hepatoma Cell Line,” Science 285:110-113 (1999), which is hereby incorporated by reference in its entirety.

### Example 90

### In vitro HCV Infection Experiment

The in vitro HCV infection experiment is performed as described by Kato et al., “Replication of Hepatitis C Virus in Cultured Non-Neoplastic Human Hepatocytes,” Jpn. J. Cancer Res. 87:787-792 (1996), which is hereby incorporated by reference in its entirety, and Ikada et al., “Human Hepatocyte Clonal Cell Lines That Support Persistent Replication of Hepatitis C Virus,” Virus Res. 56:157-167 (1998), which is hereby incorporated by reference in its entirety.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. A method of preventing or treating a mammal with a viral-induced disorder comprising:
administering to the mammal a therapeutically effective amount of a compound having the following formula: 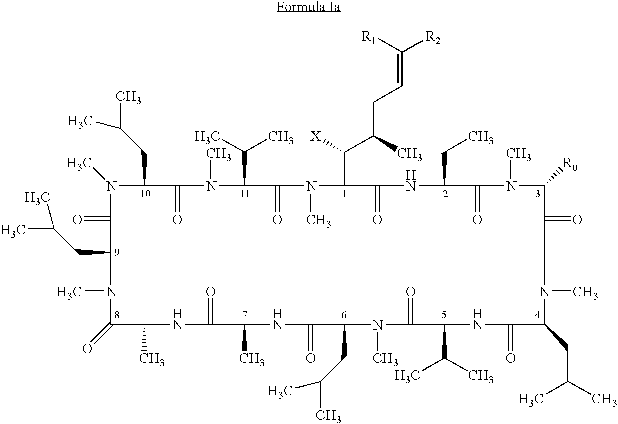
wherein:
X is OH or OAc;
R₀ is H or CH₂OR₃;
R₁ is H or D;
R₂ is selected from the group consisting of:
halogen,
C₁-C₆ halogenated saturated straight or branched carbon chain,
C₂-C₆ halogenated unsaturated straight or branched carbon chain,
C₃-C₆ substituted and unsubstituted cycloalkyl,
C₁-C₆ saturated straight or branched carbon chain containing amino group,
—CH═N—OR₄, and
—CH═N—NR₄R₅;
R₃ is selected from the group consisting of:
hydrogen,
alkanoyl,
alkenoyl,
alkynoyl,
aryloyl,
arylalkanoyl,
alkylaminocarbonyl,
arylaminocarbonyl,
arylalkylaminocarbonyl,
alkyloxycarbonyl,
aryloxycarbonyl, and
arylalkyloxycarbonyl;
R₄ and R₅ are the same or different and independently selected from the group consisting of:
hydrogen,
C₁-C₆ saturated straight or branched carbon chain,
C₃-C₆ unsaturated straight or branched carbon chain,
C₃-C₆-substituted and unsubstituted cycloalkyl,
C₁-C₄ carbon chain containing an aryl or heteroaryl, substituted and unsubstituted aryl,
substituted and unsubstituted heteroaryl, alkanoyl,
alkenoyl,
alkynoyl,
aryloyl,
arylalkanoyl,
alkylaminocarbonyl,
arylaminocarbonyl,
arylalkylaminocarbonyl,
alkyloxycarbonyl,
aryloxycarbonyl,
arylalkyloxycarbonyl,
alkylsulfonyl, and
arylsulfonyl; and
R₄ together with R₅ results in the formation of a cyclic moiety of C₂-C₆ optionally containing heteroatom or heteroatoms,
wherein the compound is a cis geometric isomer, a trans geometric isomer, or a mixture of the cis and the trans geometric isomers or a pharmaceutically acceptable salt thereof,
under conditions effective to prevent or treat the viral-induced disorder.

2. The method according to claim 1, wherein X is OH or OAc, R₀ is H, CH₂OH or CH₂OAc, and R₁ is H or D.

3. The method according to claim 2, wherein R₂ is selected from the group consisting of F, Cl, Br, and I.

4. The method according to claim 2, wherein R₂ is selected from the group consisting of CF₃, CH₂F, and CH₂Cl.

5. The method according to claim 2, wherein R₂ is selected from the group consisting of —CH═CHF, —CH═CHCl, —CH═CHBr, and —CH═CHI.

6. The method according to claim 2, wherein R₂ is selected from the group consisting of —CH═CH—C≡CH, —CH═CH—C≡C—CH₃, and —CH═CH—C≡C—CH═CH₂.

7. The method according to claim 2, wherein R₂ is cyclopropyl.

8. The method according to claim 2, wherein R₂ is selected from the group consisting of —CH═N—OH, —CH═N—OCH₃, —CH═N—OCH₂CH₃, —CH═N—NHCH₃, and —CH═N—N(CH₃)₂.

9. The method according to claim 1, wherein the viral-induced disorder is a human immunodeficiency virus-induced disorder.

10. The method according to claim 9, wherein said compound is administered in combination with antiretroviral agents selected from the group consisting of nucleoside reverse transcriptase inhibitors, nonnucleoside reverse transcriptase inhibitors, human immunodeficiency virus protease inhibitors, fusion inhibitors, and combinations thereof.

11. The method according to claim 10, wherein the nucleoside reverse transcriptase inhibitor is selected from the group consisting of Zidovudine, Didanosine, Stavudine, and Lamivudine.

12. The method according to claim 10, wherein the nonnucleoside reverse transcriptase inhibitor is selected from the group consisting of Nevirapine, Efavirenz, and Delavirdine.

13. The method according to claim 10, wherein the human immunodeficiency virus protease inhibitor is selected from the group consisting of Saquinovir, Indinavir, and Ritonavir.

14. The method according to claim 10, wherein the fusion inhibitor is Enfuvirtide.

15. The method according to claim 1, wherein the viral-induced disorder is a hepatitis C virus-induced disorder.

16. The method according to claim 15, wherein said compound is administered in combination with an interferon.

17. The method according to claim 16, wherein the interferon is interferon α2a or interferon α2b.

18. The method according to claim 16, wherein the interferon is a pegylated interferon.

19. The method according to claim 18, wherein the pegylated interferon is pegylated interferon α2a or pegylated interferon α2b.

20. A method of preventing or treating a mammal with a viral-induced disorder comprising:
administering to the mammal a therapeutically effective amount of a compound having the following formula: 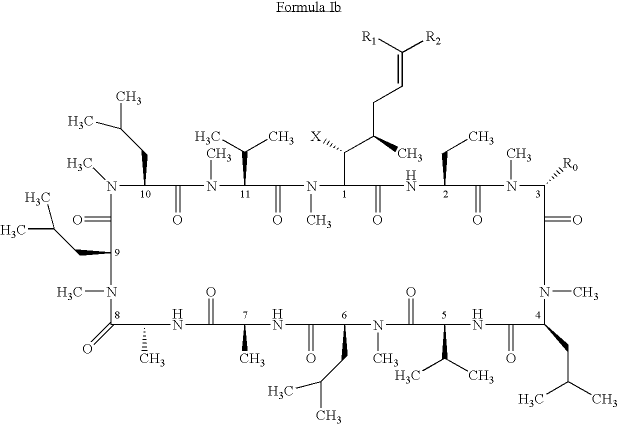
wherein:
X is OH or OAc;
R₀ is H or CH₂OR₃;
R₁ is halogen;
R₂ is selected from the group consisting of:
hydrogen,
deuterium,
halogen,
C₁-C₆ saturated straight or branched carbon chain, optionally containing halogen,
C₂-C₆ unsaturated straight or branched carbon chain, optionally containing halogen,
C₃-C₆ substituted and unsubstituted cycloalkyl, substituted and unsubstituted aryl, and
substituted and unsubstituted heteroaryl; and
R₃ is selected from the group consisting of:
hydrogen,
alkanoyl,
alkenoyl,
alkynoyl,
aryloyl,
arylalkanoyl,
alkylaminocarbonyl,
arylaminocarbonyl,
arylalkylaminocarbonyl,
alkyloxycarbonyl,
aryloxycarbonyl, and
arylalkyloxycarbonyl,
wherein the compound is a cis geometric isomer, a trans geometric isomer, or a mixture of the cis and the trans geometric isomers or a pharmaceutically acceptable salt thereof,
under conditions effective to prevent or treat the viral-induced disorder.

21. The method according to claim 20, wherein the viral-induced disorder is a human immunodeficiency virus-induced disorder.

22. The method according to claim 21, wherein said compound is administered in combination with antiretroviral agents selected from the group consisting of nucleoside reverse transcriptase inhibitors, nonnucleoside reverse transcriptase inhibitors, human immunodeficiency virus protease inhibitors, fusion inhibitors, and combinations thereof.

23. The method according to claim 22, wherein the nucleoside reverse transcriptase inhibitor is selected from the group consisting of Zidovudine, Didanosine, Stavudine, and Lamivudine.

24. The method according to claim 22, wherein the nonnucleoside reverse transcriptase inhibitor is selected from the group consisting of Nevirapine, Efavirenz, and Delavirdine.

25. The method according to claim 22, wherein the human immunodeficiency virus protease inhibitor is selected from the group consisting of Saquinovir, Indinavir, and Ritonavir.

26. The method according to claim 22, wherein the fusion inhibitor is Enfuvirtide.

27. The method according to claim 20, wherein the viral-induced disorder is a hepatitis C virus-induced disorder.

28. The method according to claim 27, wherein said compound is administered in combination with an interferon.

29. The method according to claim 28, wherein the interferon is interferon α2a or interferon α2b.

30. The method according to claim 28, wherein the interferon is a pegylated interferon.

31. The method according to claim 30, wherein the pegylated interferon is pegylated interferon α2a or pegylated interferon α2b.


SEQUENCE LISTING (1 sequence)

SEQ ID NO: 1 - protein, 4 aa, Artificial Sequence
  Description of Artificial Sequence  Peptide substrate
  MOD_RES (1): Xaa at position 1 is alanine (Ala) modified with an N-terminal blocking group, succinyl (Suc) Group
  MOD_RES (4): Xaa at position 4 is phenylalanine (Phe) modified with a C-terminal blocking group, p-nitroanilide (pNA)
XAPX